(19) United States
(12) Patent Application Publication
(10) Pub. No.: US 2025/0014681 A1
(43) Pub. Date: Jan. 9, 2025

(54) INFERRING SUPER-RESOLUTION TISSUE ARCHITECTURE BY INTEGRATING SPATIAL TRANSCRIPTOMICS WITH HISTOLOGY

(71) Applicant: The Trustees of the University of Pennsylvania, Philadelphia, PA (US)

(72) Inventors: Daiwei Zhang, Swarthmore, PA (US); Mingyao Li, Philadelphia, PA (US)

(21) Appl. No.: 18/764,858

(22) Filed: Jul. 5, 2024

Related U.S. Application Data
(60) Provisional application No. 63/525,150, filed on Jul. 5, 2023.

(51) Int. Cl. G16B 25/10; G06T 7/00

(52) U.S. Cl. CPC G16B 25/10; G06T 7/0012; G06T 2207/20081; G06T 2207/30024

(57) ABSTRACT

A method for inferring super-resolution tissue architecture by integrating spatial transcriptomics with histology includes receiving at least one histology image of a tissue sample at a histology feature extractor. The histology feature extractor partitions the at least one histology image into image tiles and partitions each of the image tiles into image sub-tiles. The histology feature extractor extracts histology features from the histology image, the extracted histology features include low-level image features extracted from the image sub-tiles and high-level image features extracted from the image tiles. A super-resolution gene expression predictor predicts gene expression for the image sub-tiles using the extracted histology features and a predictor model trained with spot-level gene expression observations. A tissue architecture annotator clusters the image sub-tiles based on the predicted gene expression of the image sub-tiles and annotates each of the image sub-tiles using the predicted gene expressions and a marker gene reference panel.

### CROSS-REFERENCE TO RELATED APPLICATIONS

This application claims the benefit of U.S. Provisional patent application Ser. No. 63/525,150, filed Jul. 5, 2023, the disclosure of which is incorporated herein by reference in its entirety.

### STATEMENT OF GOVERNMENT INTEREST

This invention was made with government support under EY030192, HG013185, and GM125301 awarded by the National Institutes of Health. The government has certain rights in the invention.

### TECHNICAL FIELD

The subject matter described herein relates to super-resolution tissue architecture. More specifically, the subject matter relates to methods, systems, and computer readable media for inferring super-resolution tissue architecture by integrating spatial transcriptomics with histology.

### BACKGROUND

The rapid advancement of spatial transcriptomics (ST) technologies has made it possible to measure gene expression within the original tissue context, enabling researchers to characterize spatial gene expression patterns, study cell-cell communications, and resolve the spatiotemporal order of cellular development. These applications have transformed our understanding of the functional organization of tissues. Despite the availability of many ST platforms, none of them provide a comprehensive solution. An ideal ST platform should offer single-cell resolution, cover the entire transcriptome, capture a large tissue area, and be cost-effective. While generating such ST data with existing platforms remains challenging, innovative computational approaches can be employed to reconstruct such data in silico.

Popular experimental methods for ST include in situ sequencing or hybridization-based technologies, such as STARmap, seqFISH, and MERFISH, and spatial barcoding followed by next-generation sequencing-based technologies, such as 10× Visium, SLIDE-seqV2, HDST, DBiT-seq, and Stereo-seq. These platforms differ in their spatial resolution and gene coverage. In situ sequencing or hybridization-based methods typically have a higher spatial resolution and sensitivity but relatively lower multiplexity for genes, whereas sequencing-based methods cover the entire transcriptome but have a lower spatial resolution, which limits their ability in studying detailed gene expression patterns.

### SUMMARY

The subject matter relates to methods, systems, and computer readable media for inferring super-resolution tissue architecture by integrating spatial transcriptomics with histology. An exemplary method for inferring super-resolution tissue architecture by integrating spatial transcriptomics with histology includes receiving, at a histology feature extractor, at least one histology image of a tissue sample. The method further includes partitioning, by the histology feature extractor, the at least one histology image into image tiles and further partitioning each of the image tiles into image sub-tiles. The method further includes extracting, by the histology feature extractor, histology features from the histology image, the extracted histology features comprising low-level image features extracted from the image sub-tiles and high-level image features extracted from the image tiles. The method further includes predicting, by a super-resolution gene expression predictor, gene expression for each of the image sub-tiles using the extracted histology features and a predictor model trained with spot-level gene expression observations. The method further includes clustering, by a tissue architecture annotator, the image sub-tiles based on the predicted gene expression of the image sub-tiles. The method further includes annotating, by the tissue architecture annotator, each of the image sub-tiles using the predicted gene expressions and a marker gene reference panel.

According to another aspect of the subject matter described herein, the method further includes predicting, by the super-resolution gene expression predictor, single cell-level gene expressions using cell segmentation masks and the predicted sub-tile level gene expressions.

According to another aspect of the method described herein, extracting the histology features from the histology image includes mapping each of the image sub-tiles into a low-level local feature vector, mapping the low-level local features vectors of the image sub-tiles within each of the image tiles into a high-level local feature vector for each of the image tiles, and mapping the high-level local feature vectors into high-level global features.

According to another aspect of the method described herein, extracting the histology features from the histology image includes using an extractor model trained by histology datasets.

According to another aspect of the method described herein, the predictor model includes a weakly supervised learning model trained with training data comprising spot-level gene expression observations.

According to another aspect of the method described herein, the spot-level gene expression is modeled as the sum of the gene expressions of the image sub-tiles inside the spot.

According to another aspect of the method described herein, annotating each of the image sub-tiles comprises determining cell type scores by averaging the super-resolution gene expressions of each cell type's marker genes for each of the image sub-tiles and attributing the cell type with the highest score to the corresponding image sub-tile when the highest score exceeds a threshold.

According to another aspect of the method described herein, the marker gene reference panel includes user-defined structures and associated marker genes received by the tissue architecture annotator for detecting user-defined structures.

According to another aspect of the subject matter described herein, the method further includes predicting cell type composition for the clusters by determining over-represented cell types within the clusters using the annotated cell types for the image sub-tiles within the clusters.

According to another aspect of the method described herein, the at least one histology image of the tissue sample includes a plurality of histology images, wherein each of the histology images is of a distinct tissue slice of the tissue sample, wherein the method includes identifying representative histology images of the distinct tissue slices, aligning gene expressions of the representative histology images, and imputing gene expressions between the representative histology images.

According to another aspect of the method described herein, the predictor model is trained with spot-level gene expression observations of at least one training subject, wherein the at least one training subject is distinct from a source of the tissue sample.

According to another aspect of the method described herein, the prediction model is trained with spot-level gene expression observations and transcriptomics data, wherein the super-resolution gene expression predictor predicts gene expression and an omic modality.

According to another aspect of the method described herein, the prediction model is trained with information sourced from a plurality of platforms.

An exemplary system for inferring super-resolution tissue architecture by integrating spatial transcriptomics with histology includes a histology feature extractor configured for receiving at least one histology image of a tissue sample, partitioning the at least one histology image into image tiles and further partitioning each of the image tiles into image sub-tiles, and extracting histology features from the histology image, the extracted histology features comprising low-level image features extracted from the image sub-tiles and high-level image features extracted from the image tiles. The system further includes a super-resolution gene expression predictor configured for predicting gene expression for each of the image sub-tiles using the extracted histology features and a predictor model trained with spot-level gene expression observations. The system further includes a tissue architecture annotator configured for clustering the image sub-tiles based on the predicted gene expression of the image sub-tiles and annotating each of the image sub-tiles using the predicted gene expressions and a marker gene reference panel.

According to another aspect of the system described herein, the super-resolution gene expression predictor is configured for predicting single cell-level gene expressions using cell segmentation masks and the predicted sub-tile level gene expressions.

According to another aspect of the system described herein, extracting the histology features from the histology image includes mapping each of the image sub-tiles into a low-level local feature vector, mapping the low-level local features vectors of the image sub-tiles within each of the image tiles into a high-level local feature vector for each of the image tiles, and mapping the high-level local feature vectors into high-level global features.

According to another aspect of the system described herein, extracting the histology features from the histology image includes using an extractor model trained by histology datasets.

According to another aspect of the system described herein, the predictor model includes a weakly supervised learning model trained with training data comprising spot-level gene expression observations.

According to another aspect of the system described herein, the spot-level gene expression is modeled as the sum of the gene expressions of the image sub-tiles inside the spot.

According to another aspect of the system described herein, annotating each of the image sub-tiles comprises determining cell type scores by averaging the super-resolution gene expressions of each cell type's marker genes for each of the image sub-tiles and attributing the cell type with the highest score to the corresponding image sub-tile when the highest score exceeds a threshold.

According to another aspect of the system described herein, the marker gene reference panel includes user-defined structures and associated marker genes received by the tissue architecture annotator for detecting user-defined structures.

According to another aspect of the subject matter described herein, the system is configured for predicting cell type composition for the clusters by determining over-represented cell types within the clusters using the annotated cell types for the image sub-tiles within the clusters.

According to another aspect of the system described herein, the at least one histology image of the tissue sample includes a plurality of histology images, wherein each of the histology images is of a distinct tissue slice of the tissue sample, wherein the system is configured for identifying representative histology images of the distinct tissue slices, aligning gene expressions of the representative histology images, and imputing gene expressions between the representative histology images.

According to another aspect of the system described herein, the predictor model is trained with spot-level gene expression observations of at least one training subject, wherein the at least one training subject is distinct from a source of the tissue sample.

According to another aspect of the system described herein, the prediction model is trained with spot-level gene expression observations and transcriptomics data, wherein the super-resolution gene expression predictor predicts gene expression and an omic modality.

According to another aspect of the system described herein, the prediction model is trained with information sourced from a plurality of platforms.

An example non-transitory computer readable medium has stored thereon executable instructions that when executed by at least one processor of at least one computer cause the at least one computer to perform steps including receiving a histology image of a tissue sample. The steps further include partitioning the histology image into image tiles and further partitioning each of the image tiles into image sub-tiles. The steps further include extracting histology features from the histology image, the extracted histology features comprising low-level image features extracted from the image sub-tiles and high-level image features extracted from the image tiles. The steps further include predicting gene expression for each of the image sub-tiles using the extracted histology features and a predictor model trained with spot-level gene expression observations. The steps further include clustering the image sub-tiles based on the predicted gene expression of the image sub-tiles. The steps further include annotating each of the image sub-tiles using the predicted gene expressions and a marker gene reference panel.

According to another aspect of the computer readable medium described herein, the steps include predicting single cell-level gene expressions using cell segmentation masks and the predicted sub-tile level gene expressions.

The subject matter described herein may be implemented in software in combination with hardware and/or firmware. For example, the subject matter described herein may be implemented in software executed by a processor. In one example implementation, the subject matter described herein may be implemented using a non-transitory computer readable medium having stored therein computer executable instructions that when executed by the processor of a computer control the computer to perform steps. Example computer readable media suitable for implementing the subject matter described herein include non-transitory devices, such as disk memory devices, chip memory devices, programmable logic devices, field-programmable gate arrays, and application specific integrated circuits. In addition, a computer readable medium that implements the subject matter described herein may be located on a single device or computer platform or may be distributed across multiple devices or computer platforms.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter described herein will now be explained with reference to the accompanying drawings of which:

FIG. 1 is a block diagram of a system for inferring super-resolution tissue architecture by integrating spatial transcriptomics with histology;

FIG. 2A shows a flow chart illustrating workflow and super-resolution gene expression prediction accuracy of a system for inferring super-resolution tissue architecture by integrating spatial transcriptomics with histology;

FIG. 2B shows a comparison of prediction accuracy between XFuse and a system for inferring super-resolution tissue architecture by integrating spatial transcriptomics with histology;

FIG. 2C shows charts comparing prediction accuracy between XFuse and a system for inferring super-resolution tissue architecture by integrating spatial transcriptomics with histology;

FIG. 2D shows a comparison between ground truth gene expression and gene expression prediction by a system for inferring super-resolution tissue architecture by integrating spatial transcriptomics with histology;

FIG. 3A compares manual annotation of tissue architecture with segmentations by XFuse and a system for inferring super-resolution tissue architecture by integrating spatial transcriptomics with histology;

FIG. 3B compares manual annotations of tissue architecture samples with segmentations by a system for inferring super-resolution tissue architecture by integrating spatial transcriptomics with histology;

FIG. 3C shows clusters of predicted cell types of tissue architecture identified by a system for inferring super-resolution tissue architecture by integrating spatial transcriptomics with histology;

FIG. 3D compares manual annotations of tissue architecture samples with segmentations by a system for inferring super-resolution tissue architecture by integrating spatial transcriptomics with histology;

FIG. 3E shows a small cancer region detected by a system for inferring super-resolution tissue architecture by integrating spatial transcriptomics with histology;

FIG. 3F shows detection of tertiary lymphoid structures (TLS) by a system for inferring super-resolution tissue architecture by integrating spatial transcriptomics with histology;

FIG. 4A shows predicted super-resolution gene expressions by iStar and XFuse compared to ground truth and spot-level gene expressions for genes in Section 1 tissue whose variances are in the 80%-100% qualities;

FIG. 4B shows predicted super-resolution gene expressions by iStar and XFuse compared to ground truth and spot-level gene expressions for genes in Section 2 tissue whose variances are in the 80%-100% qualities;

FIG. 5A shows predicted super-resolution gene expressions by iStar and XFuse compared to ground truth and spot-level gene expressions for genes in Section 1 tissue whose variances are in the 60%-80% qualities;

FIG. 5B shows predicted super-resolution gene expressions by iStar and XFuse compared to ground truth and spot-level gene expressions for genes in Section 2 tissue whose variances are in the 60%-80% qualities;

FIG. 6A shows predicted super-resolution gene expressions by iStar and XFuse compared to ground truth and spot-level gene expressions for genes in Section 1 tissue whose variances are in the 40%-60% qualities;

FIG. 6B shows predicted super-resolution gene expressions by iStar and XFuse compared to ground truth and spot-level gene expressions for genes in Section 2 tissue whose variances are in the 40%-60% qualities;

FIG. 7A shows predicted super-resolution gene expressions by iStar and XFuse compared to ground truth and spot-level gene expressions for genes in Section 1 tissue whose variances are in the 20%-40% qualities;

FIG. 7B shows predicted super-resolution gene expressions by iStar and XFuse compared to ground truth and spot-level gene expressions for genes in Section 2 tissue whose variances are in the 20%-40% qualities;

FIG. 8A shows predicted super-resolution gene expressions by iStar and XFuse compared to ground truth and spot-level gene expressions for genes in Section 1 tissue whose variances are in the 0%-20% qualities;

FIG. 8B shows predicted super-resolution gene expressions by iStar and XFuse compared to ground truth and spot-level gene expressions for genes in Section 2 tissue whose variances are in the 0%-20% qualities;

FIG. 9 shows predicted super-resolution gene expressions by iStar for three breast cancer-related genes (ESR1, ERBB2, and PGR) at various resolution enhancements;

FIG. 10A show prediction accuracy of iStar and XFuse as measured by root squared error (RMSE));

FIG. 10B show prediction accuracy of iStar and XFuse as measured by structural similarity index measure (SSIM);

FIG. 11 compares iStar predicted super-resolution gene expression patterns with corresponding histology image features;

FIG. 12 shows single-cell level gene expression predicted by iStar pseudo-Visium breast cancer data derived from Xenium data;

FIG. 13A shows accuracy of iStar and XFuse for single-cell level gene expression in-sample prediction for Section 1 as measured by RMSE;

FIG. 13B shows accuracy of iStar and XFuse for single-cell level gene expression out-of-sample prediction for Section 2 as measured by RMSE;

FIG. 14 compares tissue segmentation results by iStar and XFuse on a Xenium-derived pseudo-Visium breast cancer dataset;

FIG. 15 shows iStar segmentation with different strengths of smoothing in a Gaussian filter in sample H1 of the HER2ST breast cancer dataset;

FIG. 16 shows iStar segmentation with different numbers of clusters in sample H1 of the HER2ST breast cancer dataset;

FIG. 17 is a river plot for iStar segmentation with different numbers of clusters in sample H1 of the HER2ST breast cancer dataset;

FIG. 18A shows gene expression score for cell types B cells, CAFs, and cancer epithelial and the predicted cell type map in three consecutively cut tissue sections of Subject H in the Anderson et al. HER2ST breast cancer dataset;

FIG. 18B shows gene expression score for cell types endothelial, myeloid, and normal epithelial and the predicted cell type map in three consecutively cut tissue sections of Subject H in the Anderson et al. HER2ST breast cancer dataset;

FIG. 18C shows gene expression score for cell types plasmablasts, PLV, and T cells and the predicted cell type map in three consecutively cut tissue sections of Subject H in the Anderson et al. HER2ST breast cancer dataset;

FIG. 19A shows the most over-expressed gene in each automatically detected tissue cluster in three consecutively cut tissue sections of Subject H in the Anderson et al. HER2ST breast cancer dataset;

FIG. 19B shows the most over-expressed gene in each automatically detected tissue cluster in three consecutively cut tissue sections of Subject H in the Anderson et al. HER2ST breast cancer dataset;

FIG. 20A shows the TLS score and the super-resolution gene expression of TLS marker genes MSA1, CD3D, and CR2 in three consecutively cut tissue sections of Subject H in the Anderson et al. HER2ST breast cancer dataset;

FIG. 20B shows the super-resolution gene expression of CXCR5, CXCL13, CD4, and CD8A in three consecutively cut tissue sections of Subject H in the Anderson et al. HER2ST breast cancer dataset;

FIG. 21A shows TLSs of Subject H in the Anderson et al. HER2+ breast cancer dataset detected by iStar;

FIG. 21B shows TLSs of Subject G in the Anderson et al. HER2+ breast cancer dataset detected by iStar;

FIG. 22A shows a pathologist annotation of a breast cancer dataset;

FIG. 22B shows a total gene expression for each spot in a breast cancer dataset;

FIG. 22C shows an iStar segmentation of a breast cancer dataset;

FIG. 22D shows an iStar cell type annotation overtop histology;

FIG. 23A shows spot-level training data, ground truth gene expression, and predicted super-resolution gene expression by iStar and xFuse for different genes whose variance are in the 80%-100% qualities;

FIG. 23B shows spot-level training data, ground truth gene expression, and predicted super-resolution gene expression by iStar and xFuse for different genes whose variance are in the 80%-100% qualities;

FIG. 23C shows spot-level training data, ground truth gene expression, and predicted super-resolution gene expression by iStar and xFuse for different genes whose variance are in the 80%-100% qualities;

FIG. 24A shows spot-level training data, ground truth gene expression, and predicted super-resolution gene expression by iStar and xFuse for different genes whose variance are in the 60%-80% qualities;

FIG. 24B shows spot-level training data, ground truth gene expression, and predicted super-resolution gene expression by iStar and xFuse for different genes whose variance are in the 60%-80% qualities;

FIG. 24C shows spot-level training data, ground truth gene expression, and predicted super-resolution gene expression by iStar and xFuse for different genes whose variance are in the 60%-80% qualities;

FIG. 25A shows spot-level training data, ground truth gene expression, and predicted super-resolution gene expression by iStar and xFuse for different genes whose variance are in the 40%-60% qualities;

FIG. 25B shows spot-level training data, ground truth gene expression, and predicted super-resolution gene expression by iStar and xFuse for different genes whose variance are in the 40%-60% qualities;

FIG. 25C shows spot-level training data, ground truth gene expression, and predicted super-resolution gene expression by iStar and xFuse for different genes whose variance are in the 40%-60% qualities;

FIG. 26A shows spot-level training data, ground truth gene expression, and predicted super-resolution gene expression by iStar and xFuse for different genes whose variance are in the 20%-40% qualities;

FIG. 26B shows spot-level training data, ground truth gene expression, and predicted super-resolution gene expression by iStar and xFuse for different genes whose variance are in the 20%-40% qualities;

FIG. 26C shows spot-level training data, ground truth gene expression, and predicted super-resolution gene expression by iStar and xFuse for different genes whose variance are in the 20%-40% qualities;

FIG. 27A shows spot-level training data, ground truth gene expression, and predicted super-resolution gene expression by iStar and xFuse for different genes whose variance are in the 0%-20% qualities;

FIG. 27B shows spot-level training data, ground truth gene expression, and predicted super-resolution gene expression by iStar and xFuse for different genes whose variance are in the 0%-20% qualities;

FIG. 27C shows spot-level training data, ground truth gene expression, and predicted super-resolution gene expression by iStar and xFuse for different genes whose variance are in the 0%-20% qualities;

FIG. 28 shows prediction accuracy of iStar and XFuse as measured by RMSE and SSIM;

FIG. 29A shows a histology image of a mouse brain;

FIG. 29B shows an Allen Brain Atlas annotation of a mouse brain;

FIG. 29C shows segmentation of a mouse brain using iStar super-resolution gene expression;

FIG. 29D shows segmentation of a mouse brain using XFuse super-resolution gene expression;

FIG. 30A shows a pathologist annotation of a colorectar cancer Visium dataset;

FIG. 30B shows a total gene expression for each spot of a colorectar cancer Visium dataset;

FIG. 30C shows an iStar segmentation of a colorectar cancer Visium dataset;

FIG. 31A shows a pathologist annotation of a prostate cancer Visium dataset;

FIG. 31B shows a total gene expression for each spot of a prostate cancer Visium dataset;

FIG. 31C shows an iStar segmentation of a prostate cancer Visium dataset;

FIG. 32A shows a histology image of a prostate cancer Visium dataset;

FIG. 32B shows a total gene expression for each spot of a prostate cancer Visium dataset;

FIG. 32C shows a pathologist annotation of a prostate cancer Visium dataset;

FIG. 32D shows a clone annotation of a prostate cancer Visium dataset;

FIG. 32E shows a clonal tree of a prostate cancer Visium dataset;

FIG. 32F shows an iStar segmentation of a prostate cancer Visium dataset;

FIG. 33A shows an iStar segmentation of a kidney cancer Visium dataset;

FIG. 33B shows an iStar TLS score of a kidney cancer Visium dataset;

FIG. 33C shows a manual TLS annotation of a kidney cancer Visium dataset;

FIG. 34A shows a histology image of a mouse kidney Visium dataset;

FIG. 34B shows a total gene expression for each spot of a mouse kidney Visium dataset;

FIG. 34C shows an iStar segmentation of a mouse kidney Visium dataset;

FIG. 34D shows an anatomy of a mouse kidney Visium dataset;

FIG. 35A shows a histology image of a coronal section of a mouse brain Visium dataset;

FIG. 35B shows a total gene expression for each spot of a coronal section of a mouse brain Visium dataset;

FIG. 35C shows an iStar segmentation of a coronal section of a mouse brain Visium dataset;

FIG. 35D shows a hippocampus of a coronal section of a mouse brain Visium dataset;

FIG. 35E shows an Allen Brain Atlas annotation of a coronal section of a mouse brain Visium dataset;

FIG. 36A shows a histology image of a posterior section of a mouse brain Visium dataset;

FIG. 36B shows a total gene expression for each spot of a posterior section of a mouse brain Visium dataset;

FIG. 36C shows an iStar segmentation of a posterior section of a mouse brain Visium dataset;

FIG. 36D shows a hippocampus of a posterior section of a mouse brain Visium dataset;

FIG. 36E shows a cerebellum of a posterior section of a mouse brain Visium dataset;

FIG. 36F shows an Allen Brain Atlas annotation of a posterior section of a mouse brain Visium dataset;

FIG. 37A shows a histology image of an olfactory bulb of a mouse brain Visium dataset;

FIG. 37B shows a total gene expression for each spot of an olfactory bulb of a mouse brain Visium dataset;

FIG. 37C shows an iStar segmentation of an olfactory bulb of a mouse brain Visium dataset;

FIG. 37D shows an Allen Brain Atlas annotation of an olfactory bulb of a mouse brain Visium dataset;

FIG. 38 shows a graph comparing levels of transcriptome-wide gene coverage and special resolution of iStar and other sources;

FIG. 39 compares a goodness of fit of iStar and XFuse as measured by the in-sample spot-level RMSE and Pearson's correlation on the training;

FIG. 40A shows a goodness of fit of iStar for all datasets analyzed herein as measured by the in-sample spot-level RMSE;

FIG. 40B shows a goodness of fit of iStar for all datasets analyzed herein as measured by Pearson's correlation on the training;

FIG. 41 shows the impact of the confidence threshold for marker gene-based cell type annotation in iStar;

FIG. 42A shows a super-resolution gene expression prediction accuracy of iStar and XFuse as measured by Pearson's correlation coefficient;

FIG. 42B shows a super-resolution gene expression prediction accuracy of iStar and XFuse as measured by Pearson's correlation coefficient stratified by gene expression variance;

FIG. 43 shows super-resolution gene expression prediction accuracy of iStar and XFuse as measured by Pearson correlation coefficient;

FIG. 44 shows a workflow of iStar;

FIG. 45 shows a workflow of iStar;

FIG. 46 shows a workflow of a feature extractor;

FIG. 47 shows a workflow of a histology feature extractor;

FIG. 48 shows a workflow of a super-resolution gene expression predictor;

FIG. 49 shows a workflow of a super-resolution gene expression predictor;

FIG. 50 shows a workflow of a spot-level weakly supervised learning;

FIG. 51 shows a workflow of a tissue architecture annotator;

FIG. 52 shows a workflow of automatic annotation of tissue architecture;

FIG. 53 shows in-sample gene expression predictions of ERBB2 and PRG by Visium, Xenium, iStar, and XFuse;

FIG. 54 shows in-sample gene expression predictions of PRG by Visium, Xenium, iStar, and XFuse;

FIG. 55 shows out-of-sample gene expression predictions of ERBB2 and PRG by Xenium, iStar, and XFuse;

FIG. 56 shows an in-sample prediction accuracy of iStar and XFuse;

FIG. 57 shows an out-of-sample prediction accuracy of iStar and XFuse;

FIG. 58 shows registration-free 3D tissue segmentation by iStar;

FIG. 59 shows registration-free 3D tissue segmentation by iStar;

FIG. 60 shows an example of automatic tissue annotation by iStar;

FIG. 61 shows TLS detection from automatic tissue annotation by iStar;

FIG. 62 shows an example of precision from automatic annotation by iStar;

FIG. 63 shows an example of precision from automatic annotation by iStar;

FIG. 64A shows a flow diagram of a process for inferring super-resolution tissue architecture for a 3D tissue volume;

FIG. 64B is a flow diagram of an example method for inferring super-resolution tissue architecture for a 3D tissue volume;

FIG. 65 shows out-of-subject gene expression prediction, in-sample prediction, and the in-subject out-of-sample prediction;

FIG. 66A shows tissue segmentation using metabolomics data with enhanced spatial resolution;

FIG. 66B shows tissue segmentation using both metabolomics and transcriptomics data with enhanced spatial resolution;

FIG. 67A shows a ground truth Xenium expression;

FIG. 67B shows the SSIM of iStar predictions with and without Xenium expression;

FIG. 67C shows iStar prediction having a SSIM of 0.331 without the Xenium reference and iStar prediction having a SSIM of 0.43 with the Xenium reference; and

FIG. 68 is a flow diagram illustrating a method for inferring super-resolution tissue architecture by integrating spatial transcriptomics with histology.

### DETAILED DESCRIPTION

It will be understood that various details of the presently disclosed subject matter may be changed without departing from the scope of the presently disclosed subject matter. Furthermore, the foregoing description is for the purpose of illustration only, and not for the purpose of limitation.

Spatial transcriptomics (ST) has demonstrated enormous potential for generating intricate molecular maps of cells within tissues. The subject matter described herein includes methods, systems, and computer readable media for inferring super-resolution tissue architecture by integrating spatial transcriptomics with histology, which is also referred to herein as “iStar” (Inferring Super-resolution Tissue ARchitecture). The subject matter described herein includes an end-to-end workflow that integrates ST data and high-resolution histology images to predict spatial gene expression and characterize tissue architecture with super-resolution. The described method not only enhances gene expression resolution to near-single-cell levels in ST but also enables gene expression prediction in tissue sections where only histology images are available.

Previous studies have shown that gene expression patterns are correlated with histological image features, suggesting the possibility of predicting gene expression from histology. However, these existing methods do not fully utilize the rich cellular information provided by high-resolution histology images. In practice, a pathologist examines a histology image in a hierarchical manner. In this process, the first step is to identify a region of interest (ROI) through the examination of high-level image features that capture the global tissue structure. After a ROI is identified, low-level image features that reflect the local cellular structure of the tissue are examined. To mimic this process computationally, we propose to use a hierarchical image feature extraction approach that aims to capture both local and global tissue structures. We further develop a gene expression prediction model that predicts super-resolution gene expression by leveraging high-resolution tissue information obtained from hierarchically extracted image features. The resulting super-resolution gene expression enables cell type annotation with a near-single-cell resolution. We have implemented these procedures in the described subject matter.

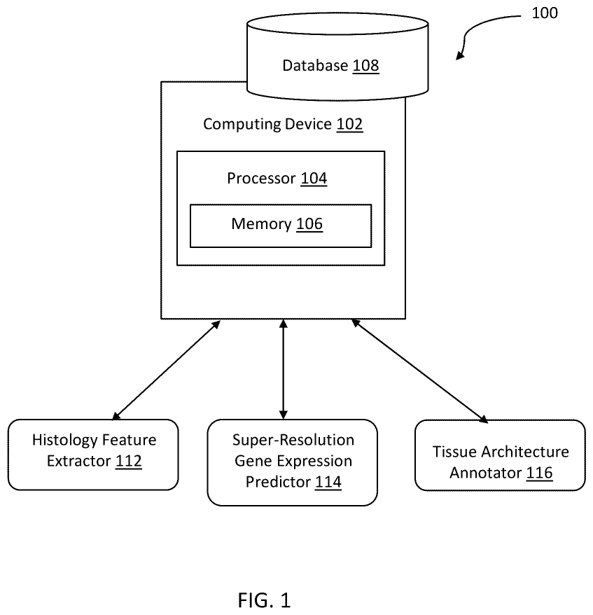

FIG. 1 is a block diagram illustrating an example system 100 for inferring super-resolution tissue architecture by integrating spatial transcriptomics with histology. System 100 may include a computing device 102 with at least one processor 104 and memory 106. Computing device 102 may include, without limitation, a microcontroller, microprocessor, digital signal processor (DSP) and/or system on a chip (SoC) as described herein. Computing device 102 may include a single computing device operating independently, or may include two or more computing devices operating in concert, in parallel, sequentially or the like; two or more computing devices may be included together in a single computing device or in two or more computing devices. Computing device 102, using processor 104 and memory 106, may be configured to perform any of the steps described herein. Computing device 102 can include a database 108 from which the computing device 102 can store, access, edit, and retrieve information. Database 108 can include at least one cloud drive. Computing device 102 is configured to communicate with a histology feature extractor 112, a super-resolution gene expression predictor 114, and/or a tissue architecture annotator 116. In some aspects of the described subject matter, histology feature extractor 112, super-resolution gene expression predictor 114, and/or tissue architecture annotator 116 are included in computing device 102.

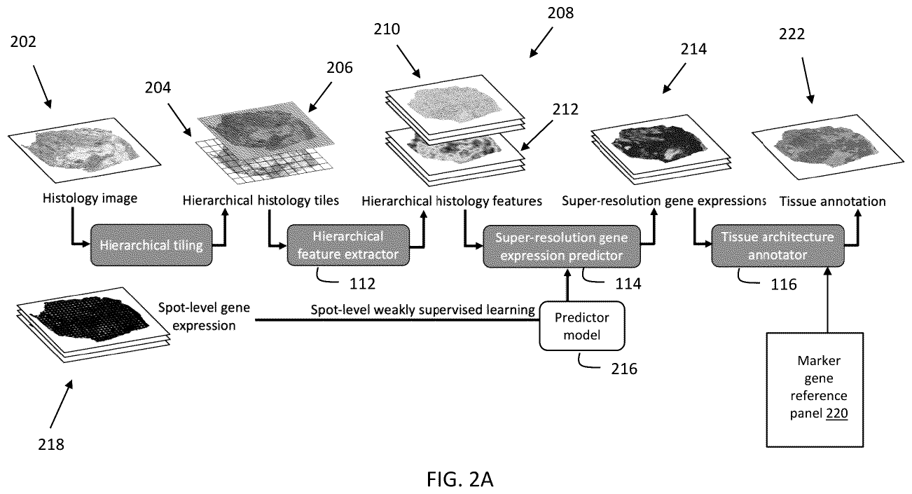

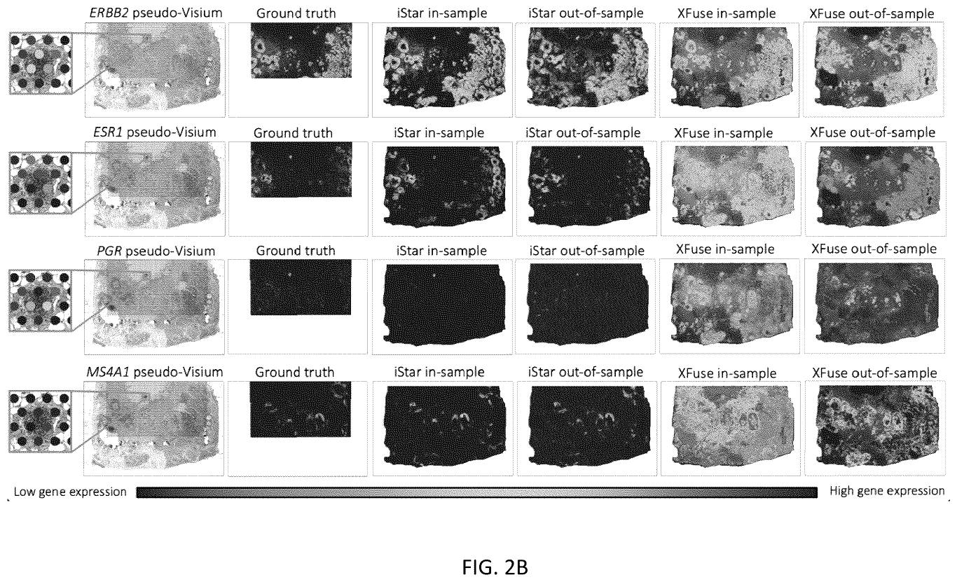

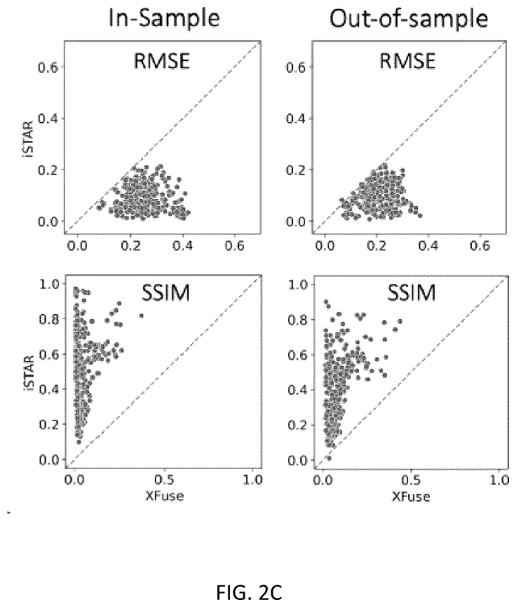

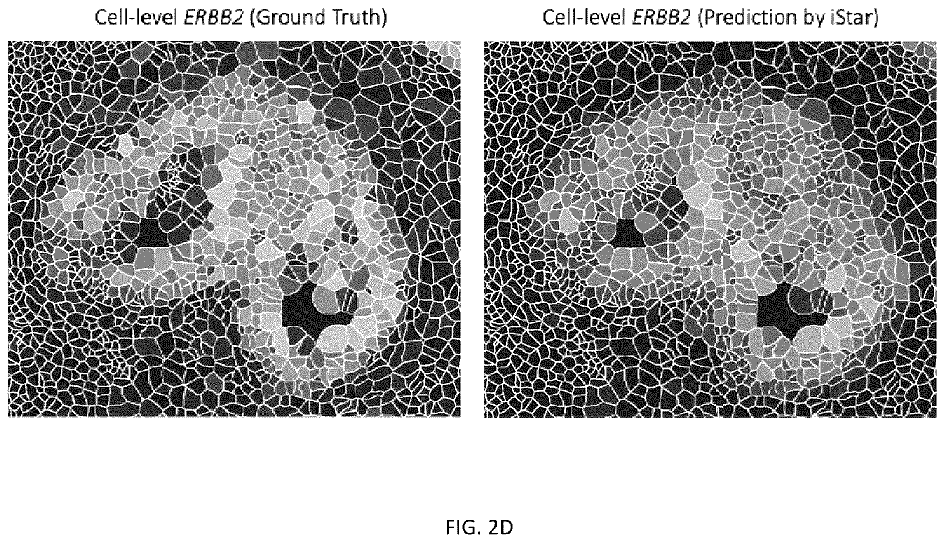

FIG. 2A shows a flow chart illustrating a workflow and super-resolution gene expression prediction accuracy of system 100, which infers super-resolution tissue architecture by integrating spatial transcriptomics with histology.

As shown in FIG. 2A, histology feature extractor 112 receives a histology image 202 of a tissue sample. Histology feature extractor 112 can be pre-trained on histology image datasets, such as datasets publicly available, using self-supervised learning. Histology images 202 can include hematoxylin-and-eosin-stained histology image datasets or any other type of histology images known in the art. Histology feature extractor 112 can include a machine learning model configured for performing visual recognition tasks, such as a hierarchical vision transformer (HVIT), that is pre-trained on the histology image datasets. Histology feature extractor 112 partitions histology image 202 into image tiles 204 and further partitioning each of the image tiles into image sub-tiles 206, which are also referred to as superpixels herein. Image tiles 204 can be any defined pixel size, such as a 256×256-pixel scale, and image sub-tiles 206 can be any division of the image tile 204 size, such as a 16×16, 32×32, or 64×64-pixel scale.

Histology feature extractor 112, specifically the HVIT model, extracts histology features 208 from histology image 202 that has been partitioned, initially extracting histology features 208 including low-level image features 210 from image sub-tiles 206 (e.g., at a 16×16-pixel scale) to capture fine-grained tissue characteristics, followed by extracting histology features 208 including high-level image features 212 from image tiles 204 (e.g., 256×256-pixel scale) to capture global tissue structures.

Subsequently, super-resolution gene expression predictor 114 predicts super-resolution gene expression 214 for each of image sub-tiles 206 using extracted histology features 208 and a predictor model 216 trained with spot-level gene expression 218 observations. Super-resolution gene expression predictor 114 utilizes low-level image features 210 from image sub-tiles 206 and high-level image features 212 from image tiles 204 with spot-level gene expression 218 data from predictor model 216 to predict super-resolution gene expressions 214. Super-resolution gene expression predictor 114 can include predictor model 216, which is a feed-forward neural network trained through weakly supervised learning based on spot-level gene expression 218 observations. Predictor model 216 divides the gene expression measurement at a given spot for each gene into multiple values, assigning one to each superpixel, facilitated by histology features 208 at every superpixel. Additionally, predictor model 216 can also predict superpixel-level gene expressions outside the spots as well as in external tissue sections, as long as histology images 202 are available.

A tissue architecture annotator 116 then clusters image sub-tiles 206 based on the predicted gene expression 214 of the image sub-tiles 206. Tissue architecture annotator 116 annotates each of image sub-tiles 206, inferring tissue architecture annotation 222, using the predicted gene expressions 214 and a marker gene reference panel 220.

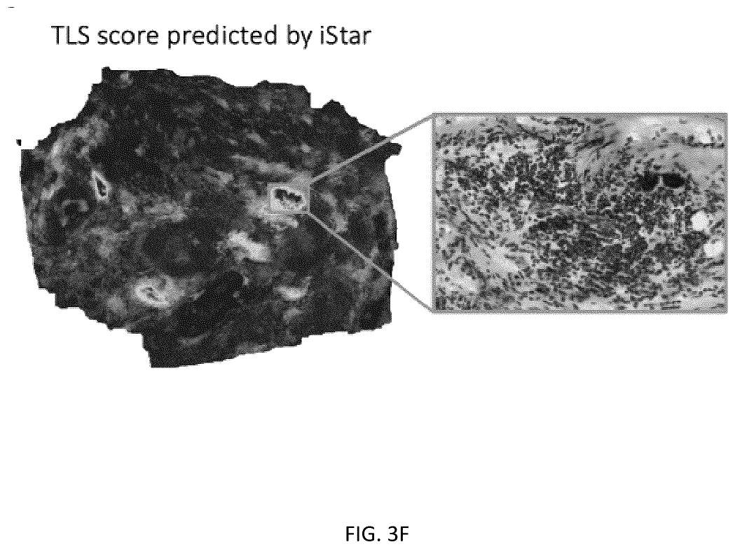

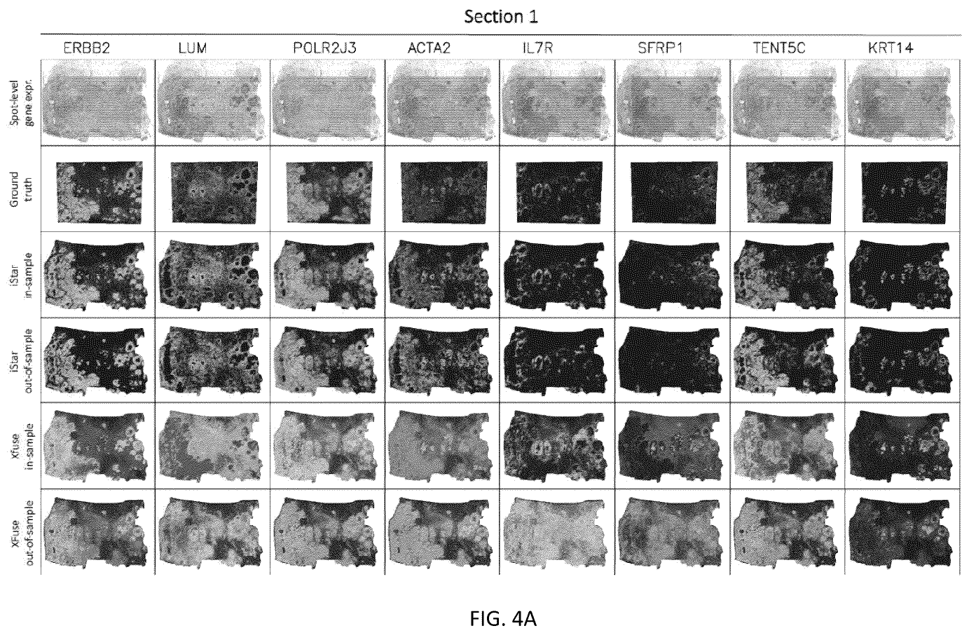

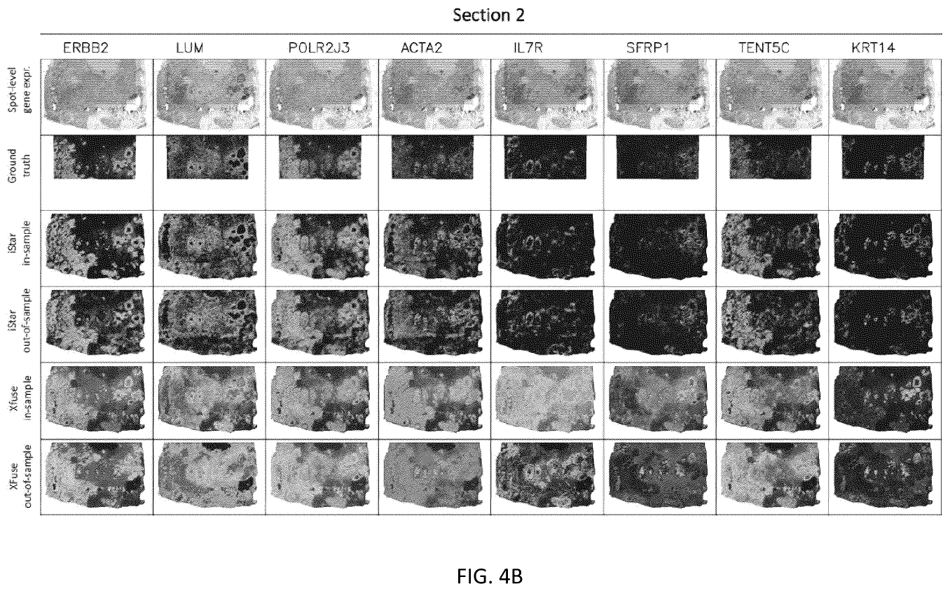

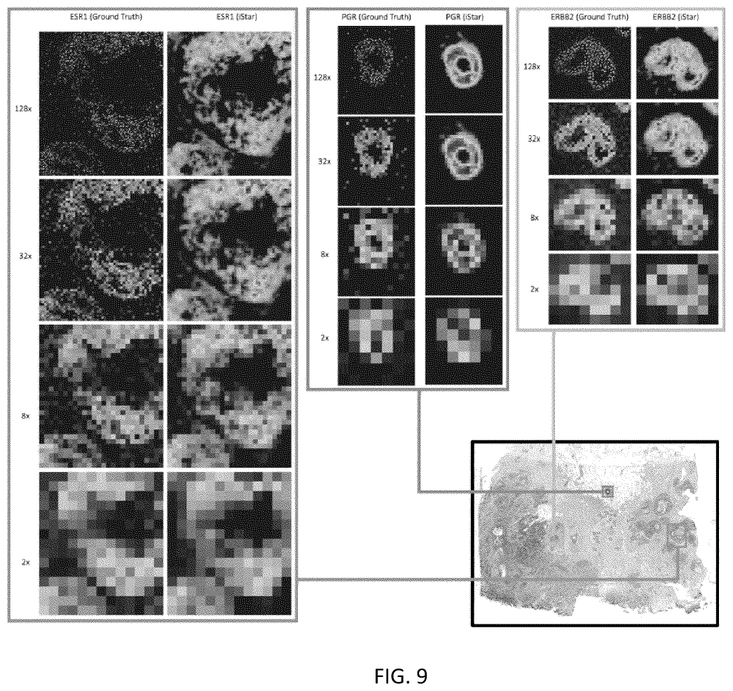

FIG. 2B shows a comparison of prediction accuracy between XFuse and system 100, also referred to herein as iStar. To assess the accuracy of iStar in super-resolution gene expression prediction, we applied it to a simulated dataset derived from the Xenium breast cancer dataset recently released by 10× Genomics. The Xenium dataset comprises sub-cellular ST data for 313 genes, measured in two consecutively cut tissue sections (i.e., Section 1 and Section 2) from a single patient. The Xenium data served as the ground truth and were used to simulate spot-level gene expression based on the spot size and layout of Visium. To simulate low-resolution Visium data, we binned the Xenium gene expressions based on Visium's spot size and layout. We assessed prediction accuracy for both in-sample and out-of-sample predictions. For in-sample prediction, model training and super-resolution gene expression prediction was performed on Section 2's pseudo-Visium data. For out-of-sample prediction, the pseudo-Visium data from Section 1 was used as the training data, and super-resolution gene expression prediction was performed on Section 2 using only its histology image as the input. We compared the prediction accuracy of iStar to that of the state-of-the-art method XFuse, and visually, iStar's predictions more closely match the ground truth as measured by Xenium compared to XFuse, as shown in FIG. 2B. FIG. 2B provides a visual comparison between iStar and XFuse. ERBB2, ESR1, and PGR are genes that encode biomarkers for breast cancer prognosis, while MS4A1 is a B cell marker gene. Super-resolution gene expressions are visualized at the scale of 8× resolution enhancement. Visualizations of additional genes at the scale of 8× resolution enhancement are presented in FIGS. 4-8. Visualizations at other resolutions are available in FIG. 9.

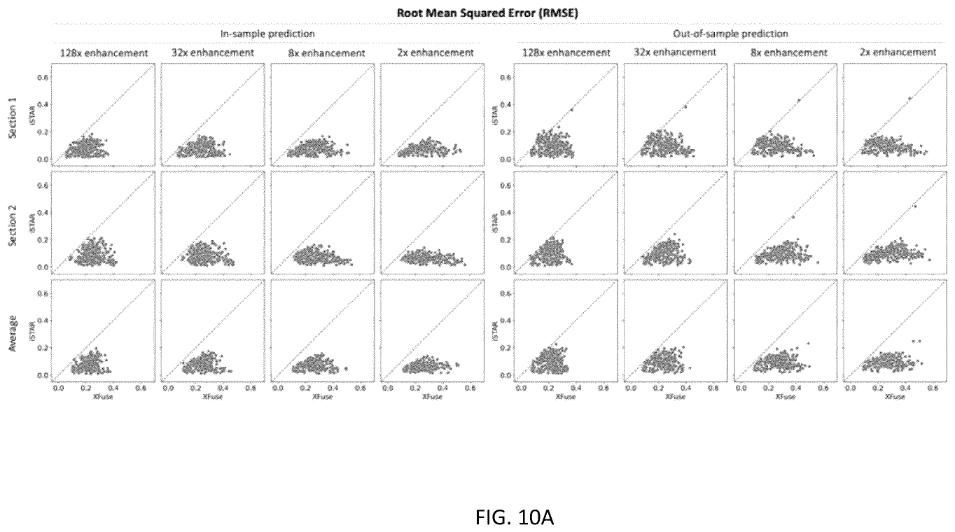

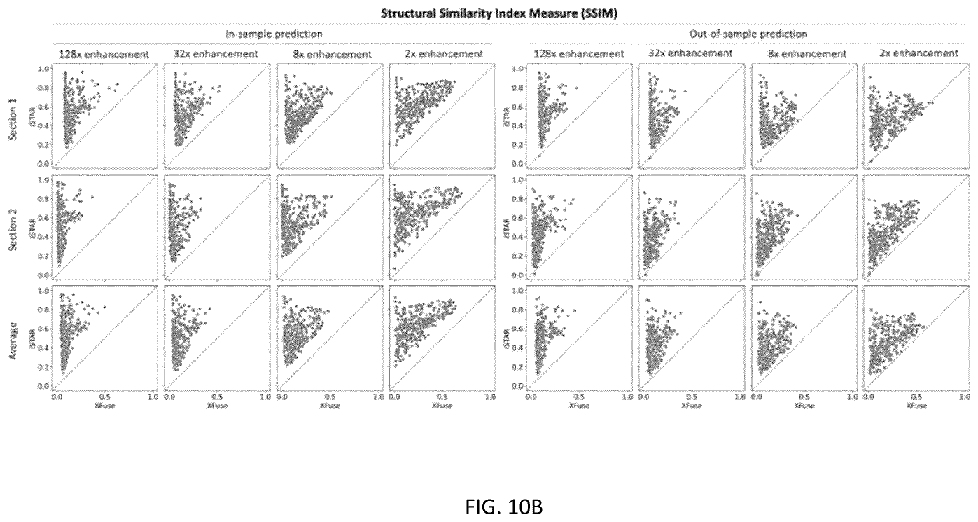

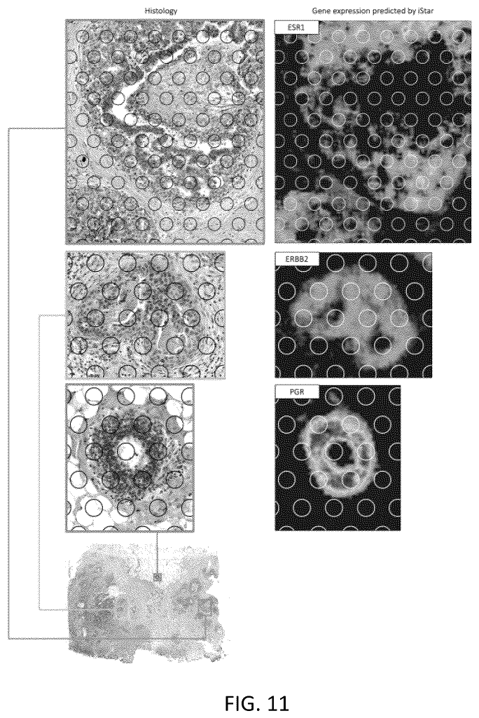

FIG. 2C shows charts comparing prediction accuracy between XFuse and iStar for 128× resolution enhancement of gene expression. The degree of resolution enhancement is defined as the number of superpixels in the super-resolution prediction divided by the number of spots in the training data. Each dot represents one of the 313 genes. Additional numerical evaluations are reported in FIG. 10. To compare the numerical performance of the two methods, we calculated the root mean square error (RMSE) and structural similarity index measure (SSIM) between the predicted and ground truth gene expressions for each gene. Our method outperformed XFuse for virtually all genes across all resolutions, as shown in FIG. 2C and FIGS. 4-10. As shown in FIG. 11, iStar predicted super-resolution gene expression pattern agrees well with the underlying histology image features. iStar not only enhances the resolution of gene expression within the measured spots but also predicts high-resolution gene expression outside the measured spots, such as in the tissue gaps between spots and on adjacent tissue sections where only the histology image is available, all with significantly higher prediction accuracies than XFuse. We further assessed iStar's ability to predict single-cell level gene expression. As shown in FIG. 2D and FIGS. 12 and 13, iStar predicted gene expression resembles that directly measured by Xenium. FIG. 2D shows a comparison between ground truth gene expression and gene expression prediction by system 100, as shown in FIG. 2A. iStar predicted single cell-level gene expression, which was computed from the predicted superpixel-level gene expression using the cell segmentation masks provided in the dataset.

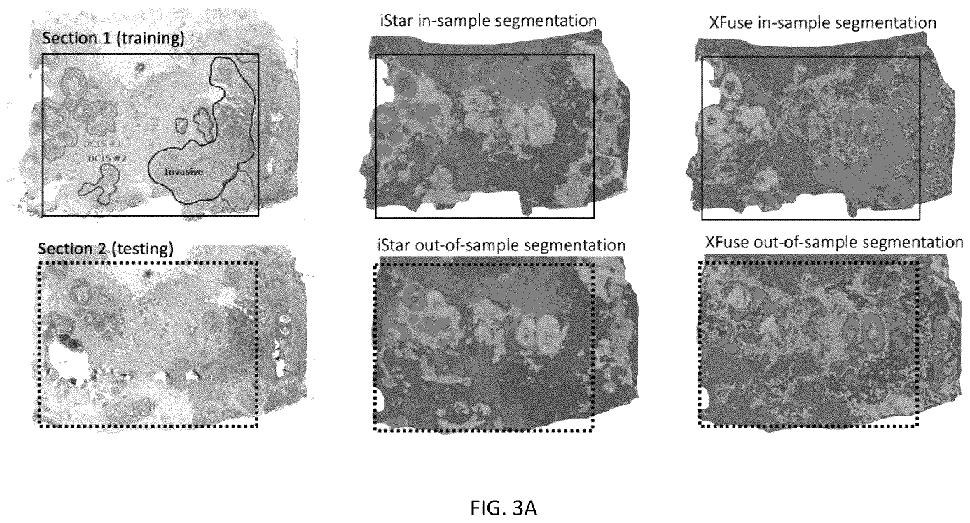

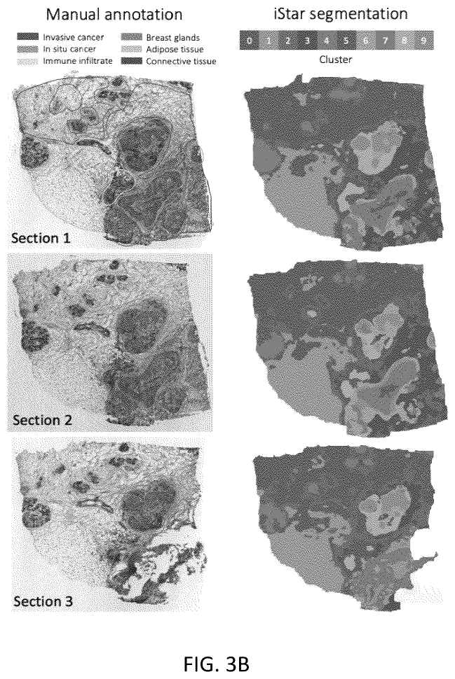

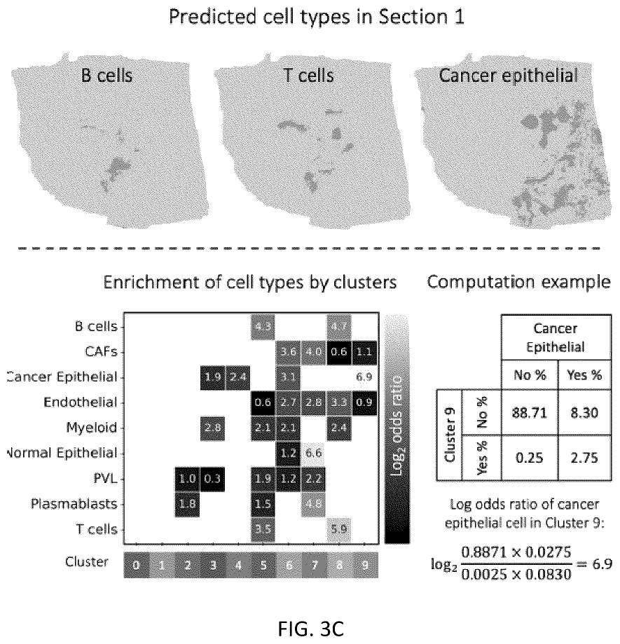

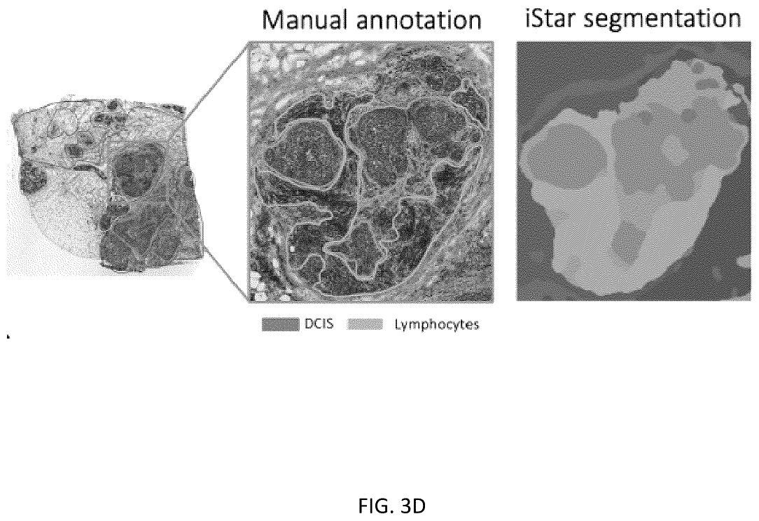

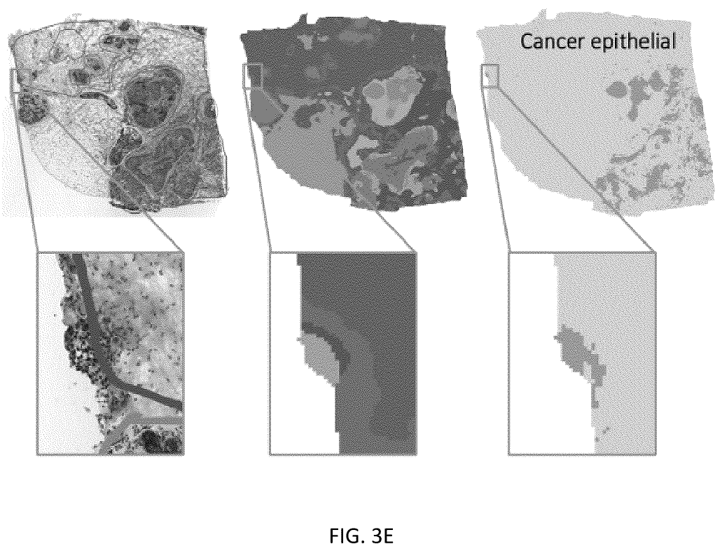

Next, we assessed iStar's capability for high-resolution annotation of tissue architecture of multiple tissue sections. FIG. 3A compares manual annotation of tissue architecture, of one of the two consecutively cut tissue sections of a breast cancer patient in the Xenium dataset with segmentations, by XFuse and iStar. The model was trained using the pseudo-Visium spot-level gene expression simulated from Section 1 (in −263 sample) of the Xenium data. Section 2 was treated as the out-of-sample section, and its super-resolution gene expression was predicted only using its histology image. Super-resolution was performed with 128× resolution enhancement.

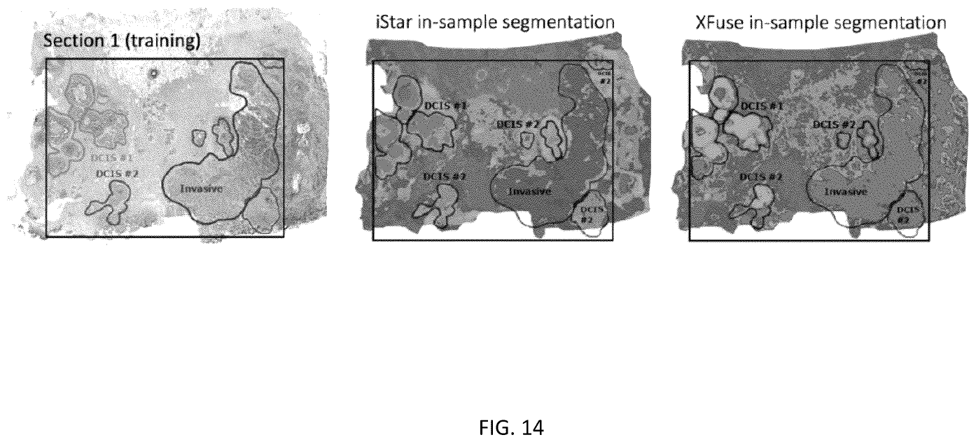

In contrast to existing frameworks, which often involve challenging image registration tasks, we show that iStar can bypass the image registration step and facilitate the annotation of tissue architecture of multiple samples. To illustrate this capability, we used the breast cancer data analyzed in FIG. 2 as an example, assuming pseudo-Visium training data were available for Section 1 but not for Section 2. To perform super-resolution gene expression prediction for the two sections, we concatenated their histology images into one and treated it as a single image in downstream analyses to perform super-resolution gene expression prediction for both sections. We used the second last layer of the feed-forward neural network as gene expression embeddings, which were treated as features for tissue segmentation. Segmentation was performed by clustering the superpixels using the k-means algorithm. Our automatic segmentation highly agreed with the manual annotation and successfully separated invasive cancer (cluster Brown), ductal carcinoma in situ (DCIS) #1 (cluster Grey), and DCIS #2 (cluster Cyan) from the rest of the tissue, as shown in FIG. 3A and FIG. 14. By contrast, segmentation using super-resolution gene expression predicted by XFuse failed to separate DCIS #2 from invasive cancer or DCIS #2 from DCIS #1. Moreover, iStar was able to annotate tissue regions outside the spot-covered tissue area, which makes it useful in tissue architecture inference for the whole tissue. Finally, the annotation for Section 2 closely resembled that of Section 1, demonstrating iStar's consistency across multiple samples. These results highlight the effectiveness of our tissue annotation procedure.

FIG. 3B compares manual annotations of tissue architecture samples with segmentations by iStar. To evaluate iStar's generalizability in super-resolution tissue segmentation and annotation, we applied it to another breast cancer dataset (denoted as HER2ST). This dataset comprises HER2+ breast cancer samples generated using the legacy Spatial Transcriptomics technology, which has a lower spatial resolution than Visium. We considered three consecutively cut tissue sections from a single patient (Subject H), where manual annotation was provided for only one section in the original publication. The manual annotation included identifying invasive cancer, in situ cancer, immune infiltrate, breast glands, adipose tissue, and connective tissue in the one section. To segment all the three sections, we carried out multi-sample tissue segmentation using the same approach as in FIG. 3A. The automatic segmentation by iStar showed strong agreement with the coarse manual annotation while providing increased granularity, as shown in FIG. 3B and FIGS. 15-17. Moreover, the three tissue sections exhibited similar structures, demonstrating the consistency of our method across multiple samples. Super-resolution was performed with 128× resolution enhancement.

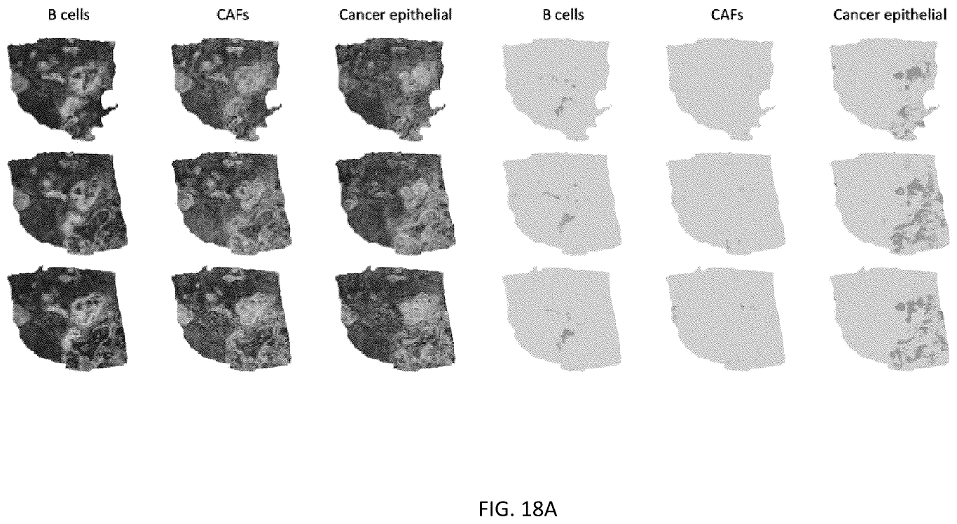

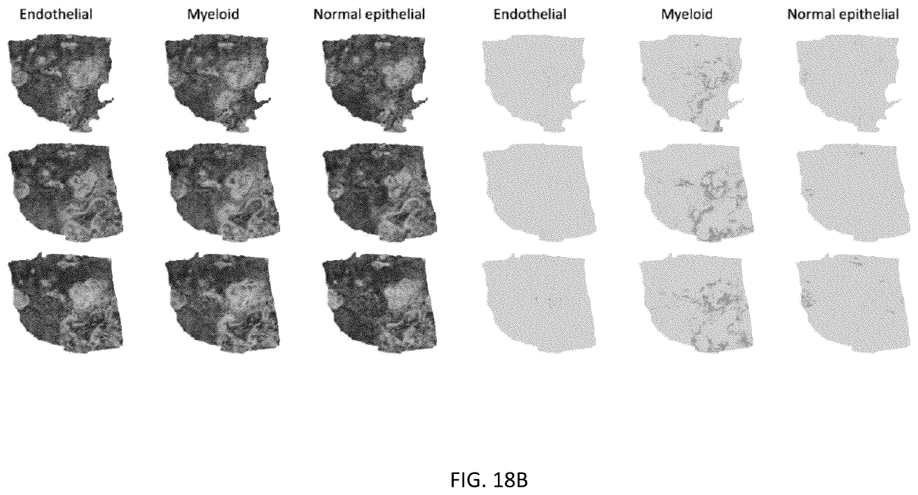

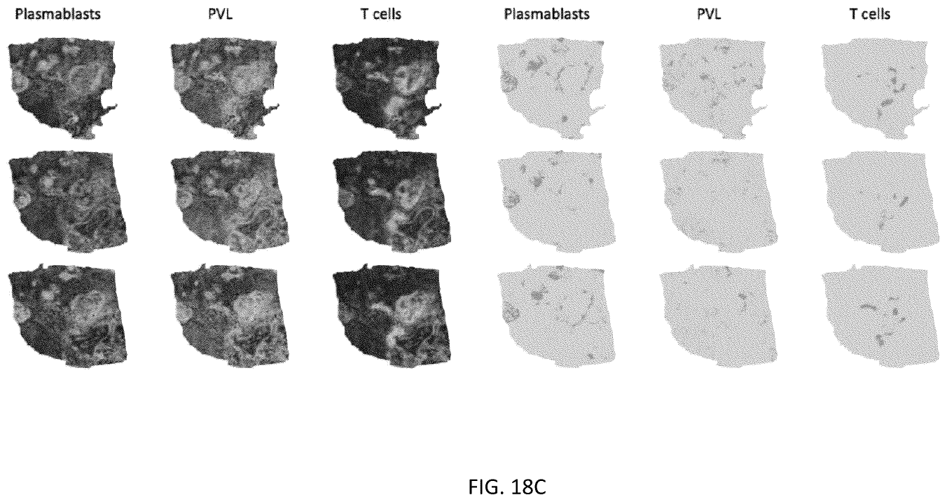

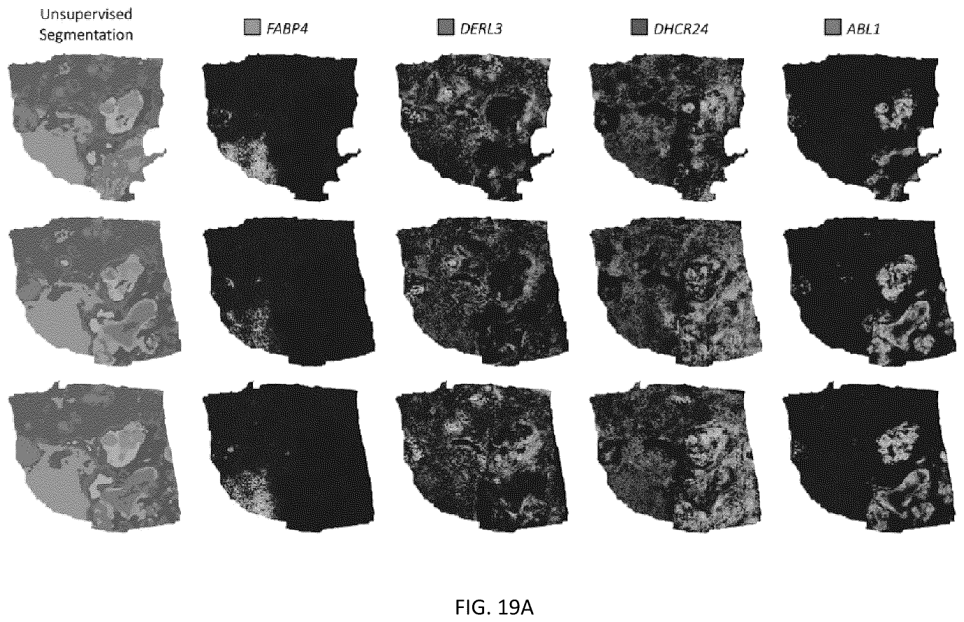

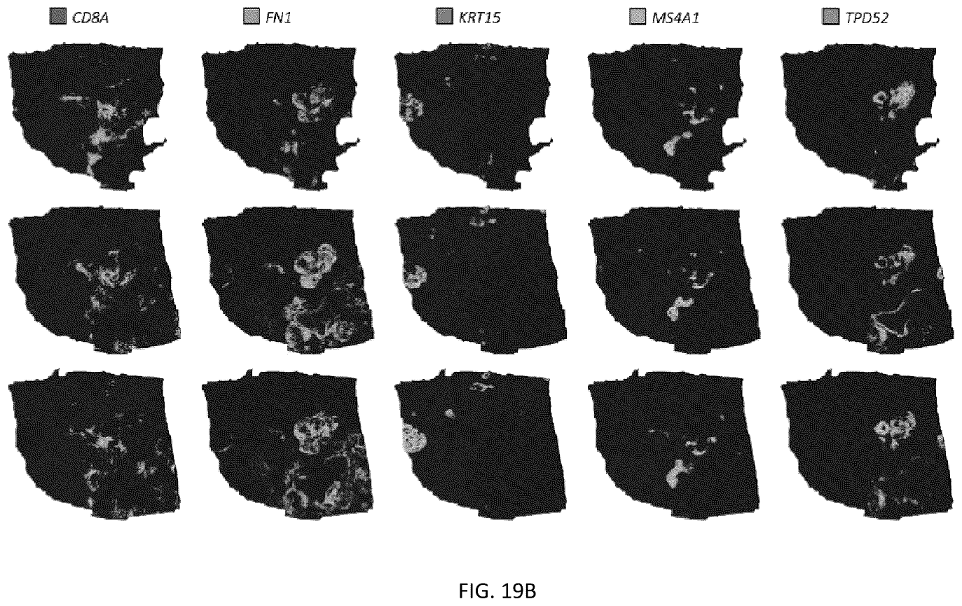

After segmenting the tissue, we conducted cell-type annotation at the superpixel level, as shown in FIG. 3C. iStar assigned biologically meaningful labels to the tissue clusters by performing superpixel-level cell type inference, followed by a cell type enrichment analysis, where depletion, i.e., negative enrichment, was not shown in the heatmap. FIG. 3C shows clusters of predicted cell types of tissue architecture identified by iStar including B cells, cancer-associated fibroblasts (CAFs), cancer epithelial, endothelial, myeloid, normal epithelial, periventricular leukomalacia (PVL), plasmablasts, and T cells. In this process, we treated each superpixel as an artificial cell and inferred its cell type based on predicted gene expressions and a reference panel of marker genes. The cell type annotation yielded an estimate of cell type proportions within each tissue cluster, enabling us to evaluate the extent of cell type enrichment in each cluster. As shown in FIG. 3C, Clusters 9 (cyan), 6 (pink), and 4 (purple), and 3 (red) are enriched with cancer epithelial cells, and these clusters closely matched with the invasive and in situ cancer regions based on the manual annotation. Furthermore, Clusters 8 (yellow) and 5 (brown) were enriched with B cells and T cells, as expected from the manual annotation. FIG. 3C visualizes the superpixels annotated as B cells, T cells, and cancer epithelial cells, with the prediction maps for the other cell types displayed in FIG. 18. In addition to cell type inference and enrichment analysis, the underlying biological relevance of each tissue cluster from unsupervised segmentation is also hinted at by the most over-expressed genes in the cluster. For example, FABP4 (fatty acid binding protein) was enriched in Cluster 1 (orange), CD8A (a lineage marker of T cell) was enriched in Cluster 5 (brown), and MS4A1 (a lineage marker of B cell) was enriched in Cluster 8 (yellow), as shown in FIGS. 19A and 19B.

FIG. 3D compares manual annotations of tissue architecture samples with segmentations by iStar. Further examination of the iStar unsupervised segmentation and the histology image revealed intratumoral heterogeneity of cancer cells that agreed with the pathologist's manual annotation, as shown in FIG. 3D, where the refined annotation was provided by a board-certified pathologist (E. E. F). Overall, superpixel-level cell type annotation provides biologically meaningful interpretations of the automatically detected tissue clusters, closely aligned with manual labels while revealing fine tissue structures. Notably, iStar was even able to detect a small cancer region, as shown in FIG. 3E, missed in the original manual annotation, and the validity of this cancer region was confirmed by E. E. F. This example demonstrates that iStar can identify small regions of interest that are easily neglected during the initial manual annotation process. Our findings in this HER2ST dataset suggest that iStar can accurately annotate tissue architecture even for the low-resolution data generated by the legacy Spatial Transcriptomics platform. The identification of biologically relevant genes within tissue clusters further supports the potential utility of iStar in uncovering new insights into tissue biology and diseases.

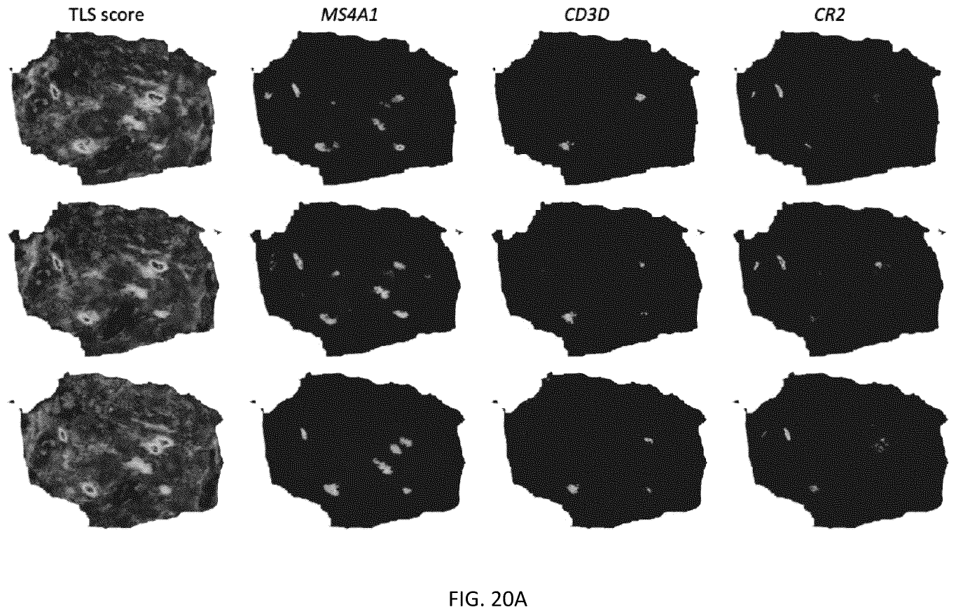

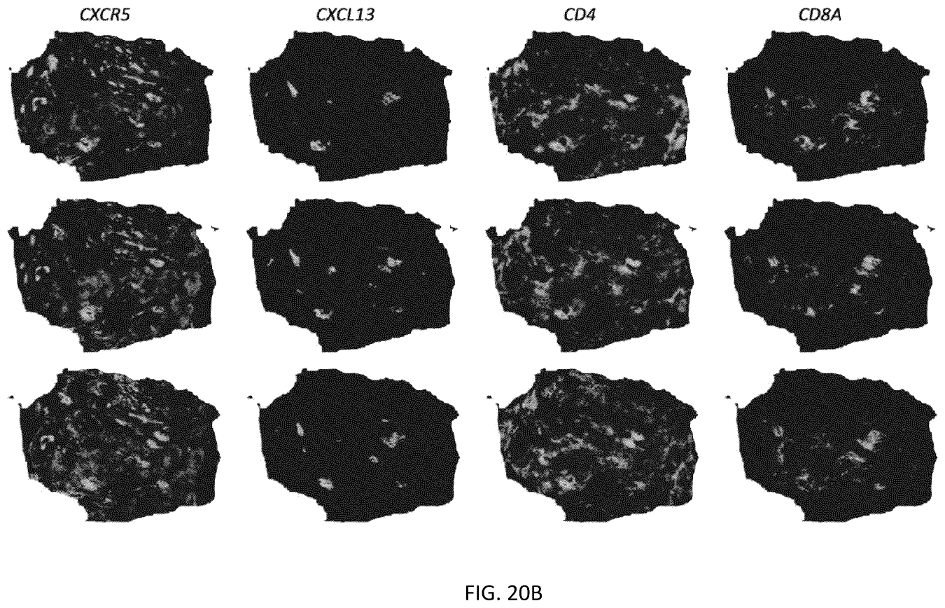

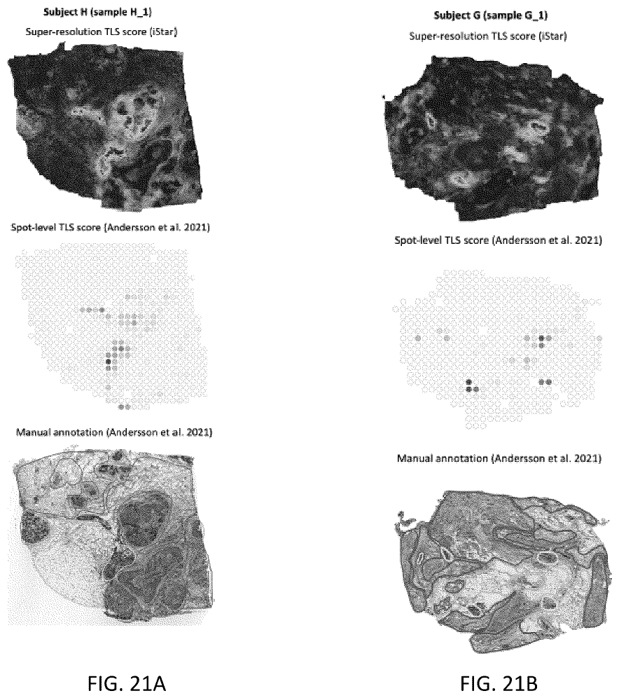

Next, we show that iStar can be utilized to detect multicellular structures, such as tertiary lymphoid structures (TLSs), which are clusters of highly organized immune cells formed in non-lymphoid tissues, often found at sites of inflammation, including a variety of solid tumors. The presence of TLSs has been shown to be associated with positive clinical outcomes and responses to immunotherapy. However, the manual detection of TLSs using the spot-resolution Visium data is labor-intensive, time-consuming, and imprecise, due to the small size and the fine-grained characteristics of TLSs. To demonstrate the ability of iStar for automatic TLS detection, we analyzed three consecutively cut tissue sections of another patient (Subject G) in the HER2ST dataset. In order to efficiently detect TLSs, we curated a list of unique TLS marker genes, as shown in Table 2, and computed TLS gene signature scores by standardizing and averaging the predicted gene expression of our curated TLS marker genes, as shown in FIG. 3F. We identified multiple TLSs in these tissue sections, all of which were confirmed by a board-certified pathologist (E. E. F.), and the TLS marker gene expressions are shown in FIG. 20. By contrast, the original HER2ST study detected several TLSs, but the analyses were based on low-resolution spot-level gene expression, resulting in a much lower resolution compared to our results, as shown in FIG. 21.

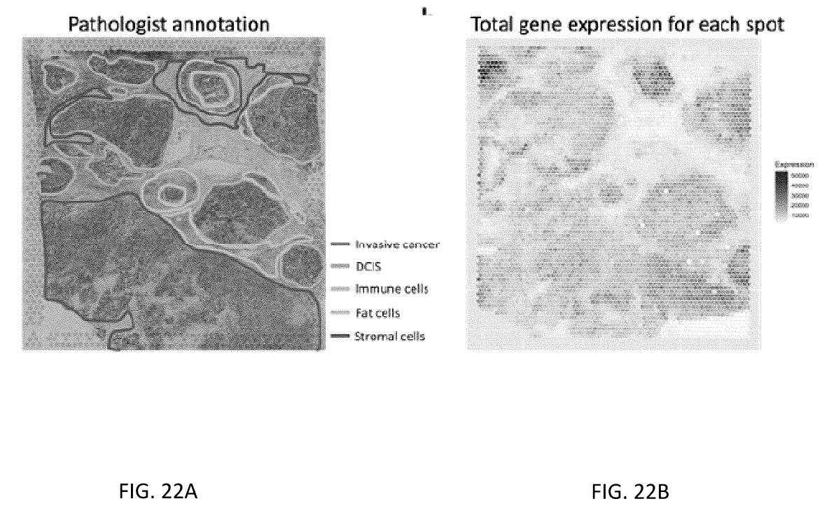

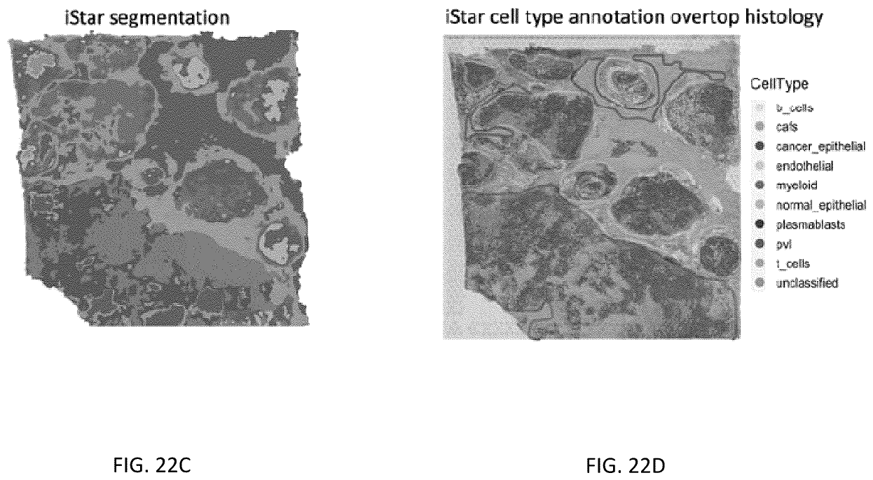

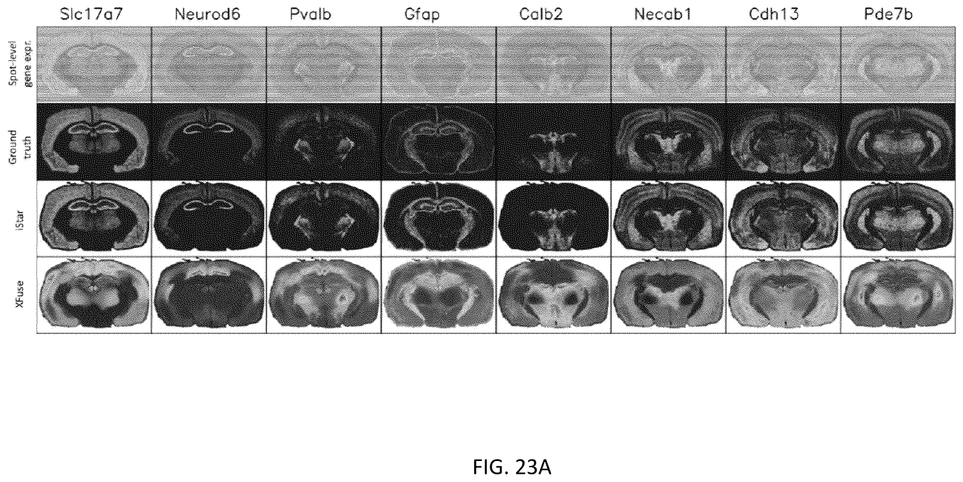

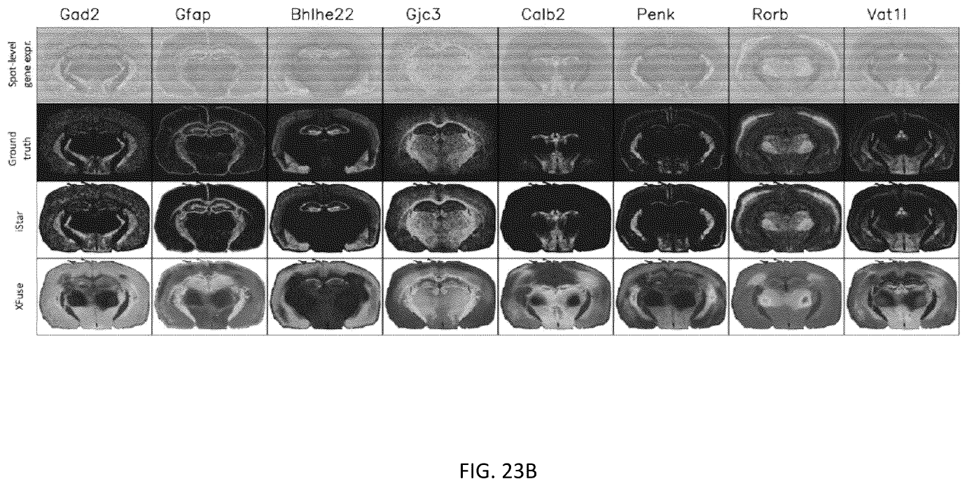

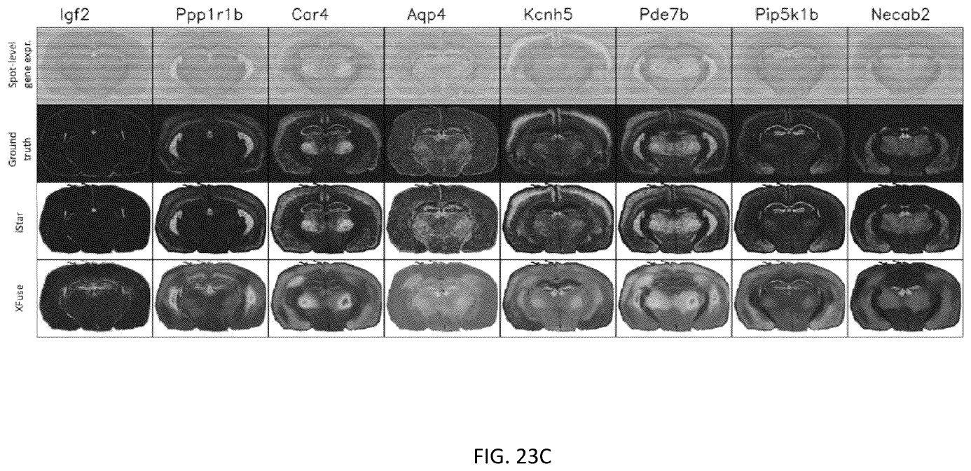

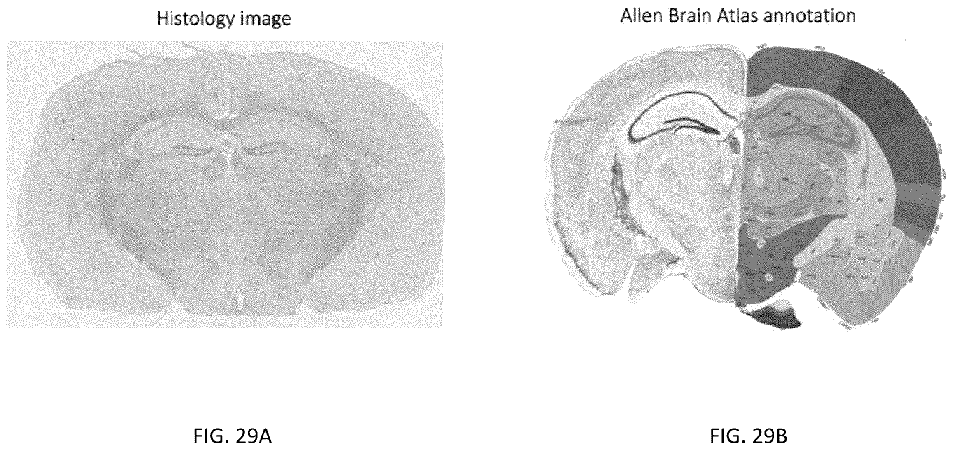

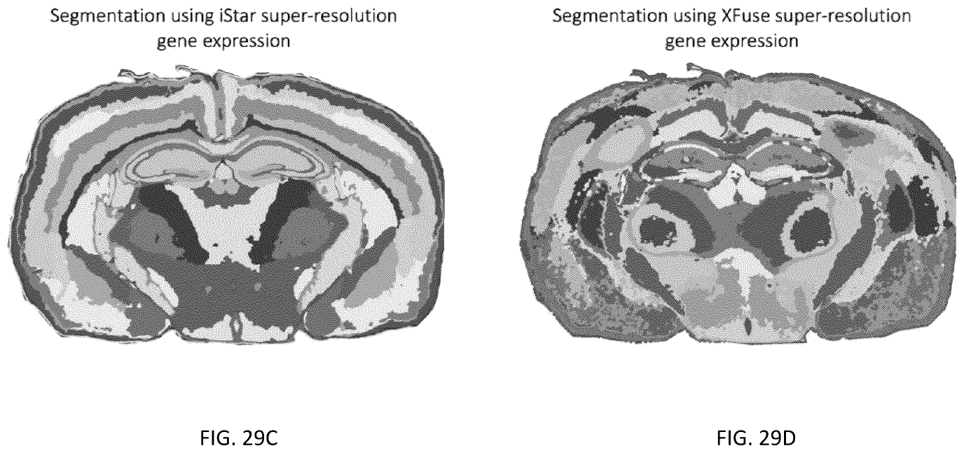

In addition to the two breast cancer datasets shown in FIGS. 2 and 3, we also analyzed one additional breast cancer dataset generated using Visium by 10× Genomics. As shown in FIG. 22, iStar revealed fine-grained tissue structures. Although we have primarily focused on the applications to breast cancer in this study, iStar is a generic tool that can be applied to various diseased or healthy tissue types. To demonstrate iStar's capability in analyzing healthy tissues, we conducted benchmarking evaluations using the recently released Xenium data generated from the mouse brain by 10× Genomics. The benchmarking was designed similarly to the experiments for the Xenium-derived pseudo-Visium breast cancer dataset in FIGS. 2 and 3. As shown in FIGS. 23-28, iStar achieved high accuracy in this evaluation across all resolutions and outperformed XFuse. In addition, our super-resolution gene expression-based segmentation, shown in FIG. 29, revealed a fine-grained tissue structure that matches closely with the Allen Brain Atlas annotation.

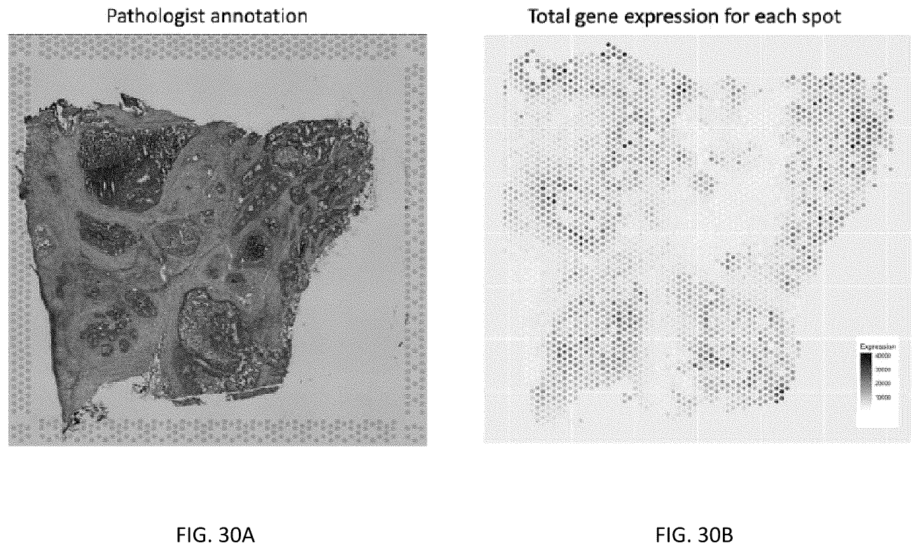

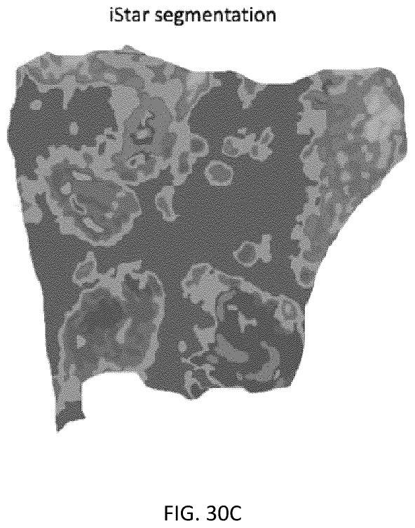

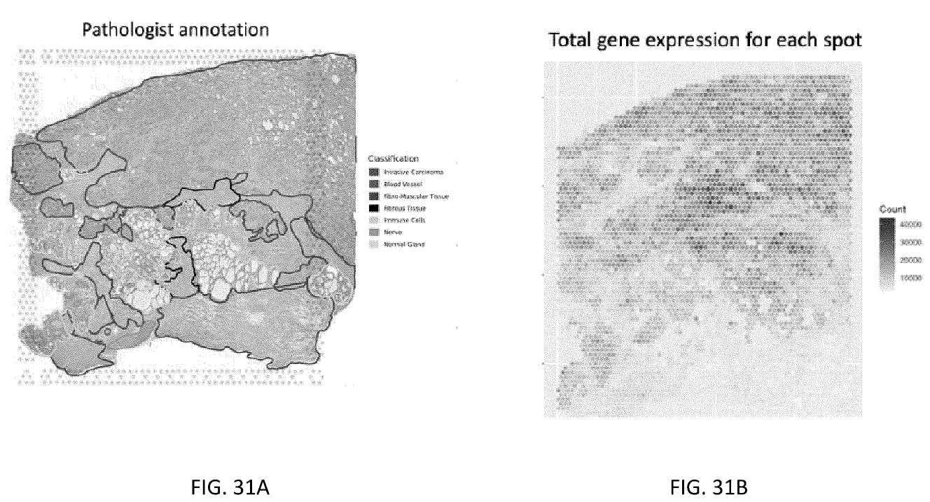

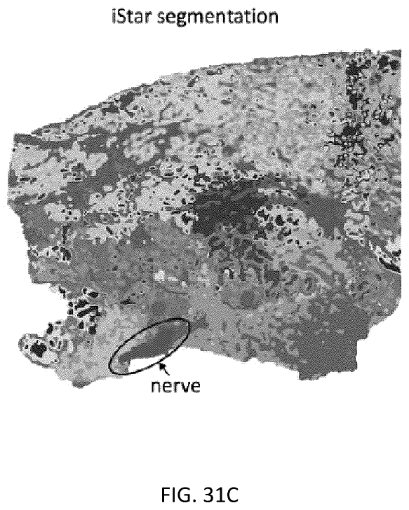

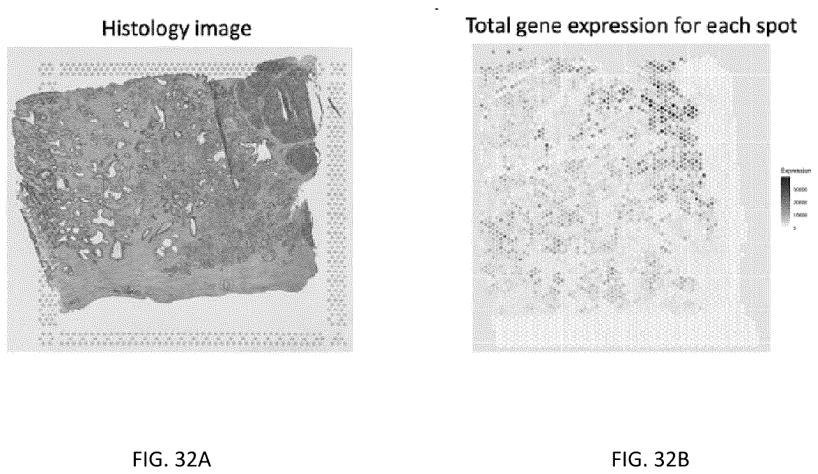

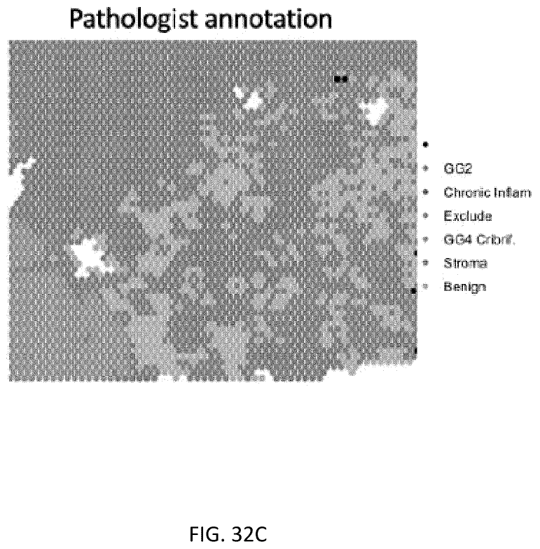

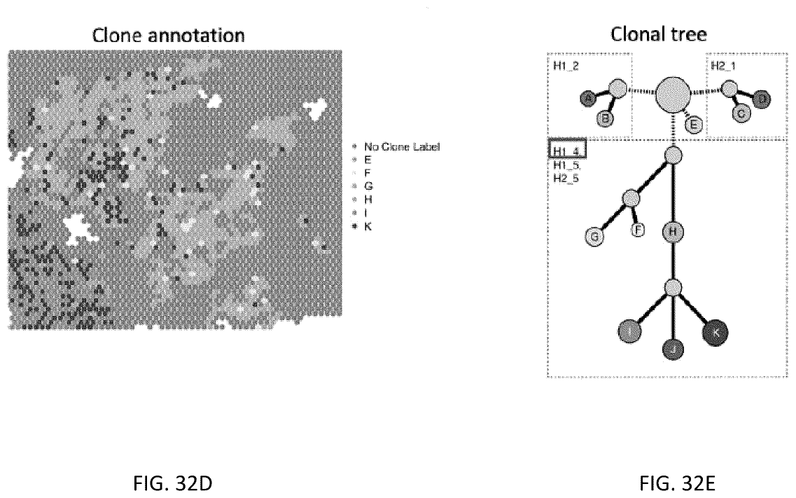

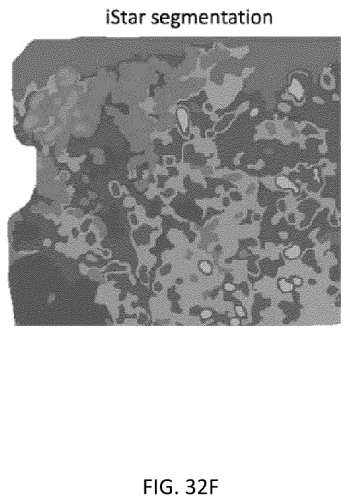

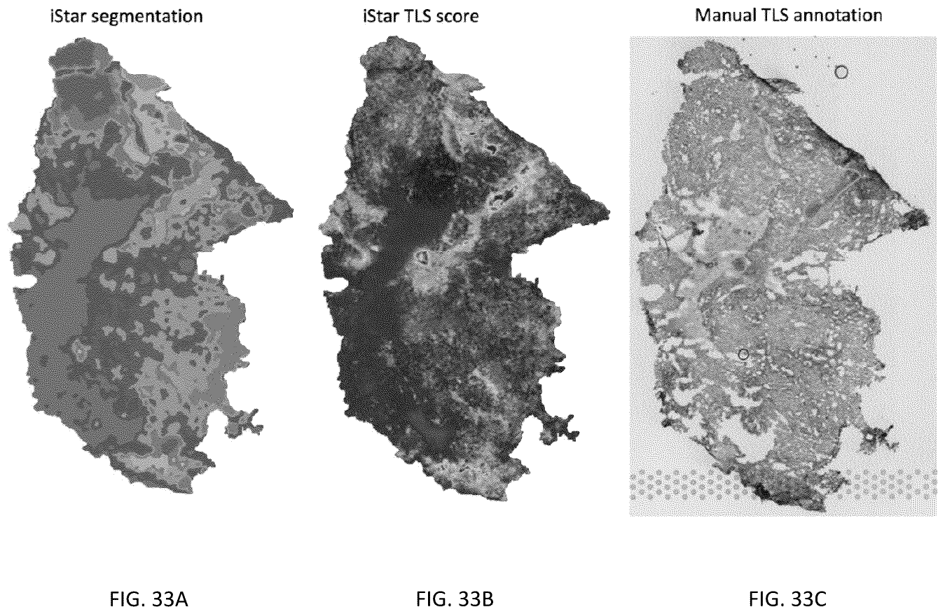

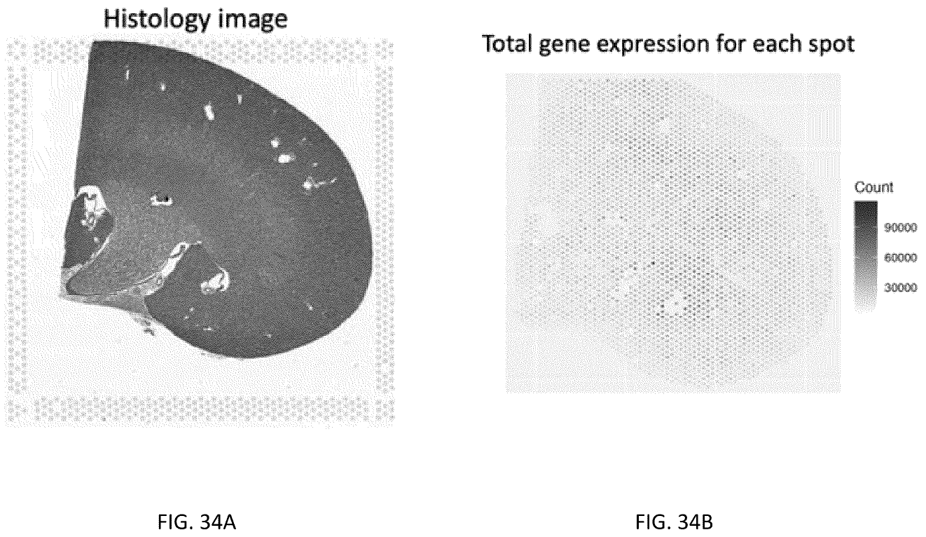

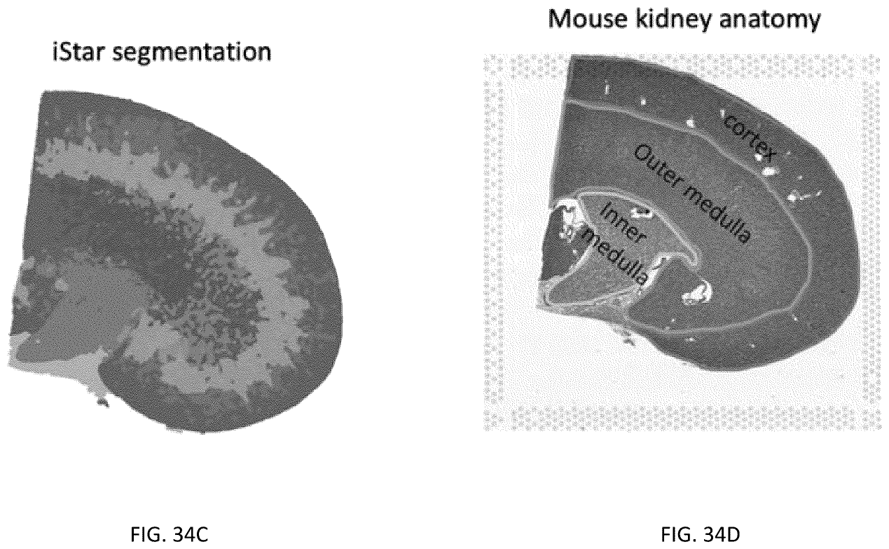

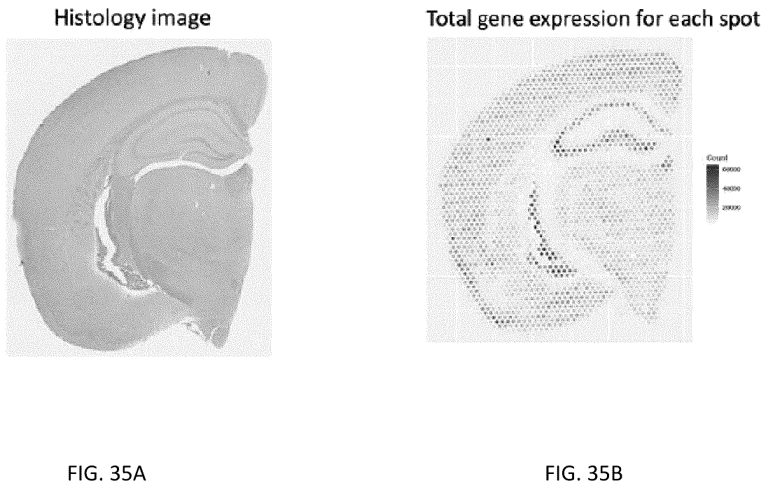

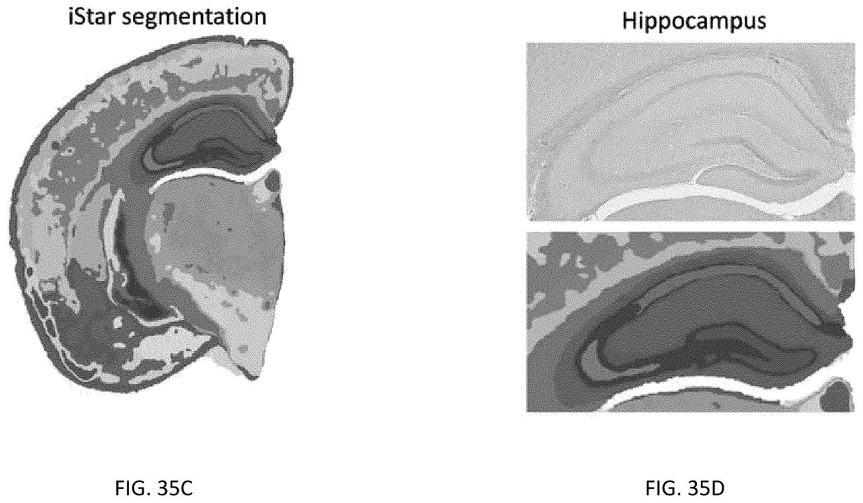

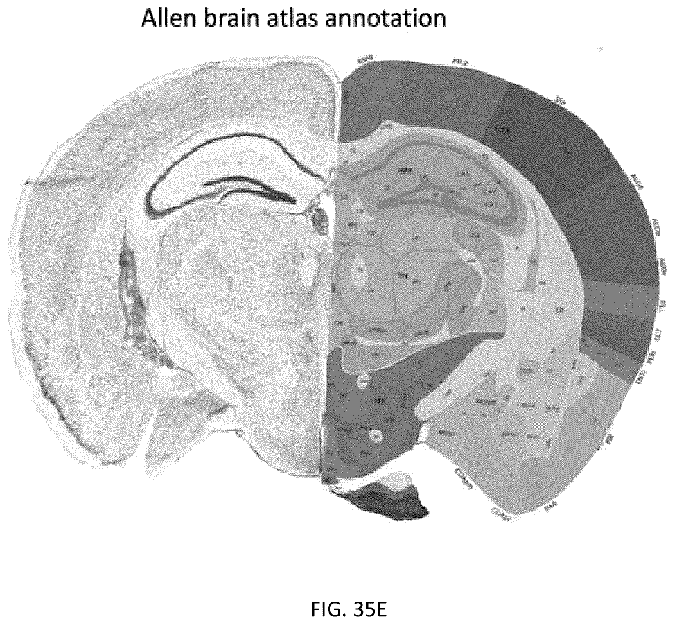

Finally, to demonstrate iStar's broad applicability to diverse cancer and healthy tissue types, we applied it to Visium data from colorectal cancer (FIG. 30), prostate cancer (FIGS. 31 and 32), kidney cancer (FIG. 33), mouse kidney (FIG. 34), and mouse brain (FIGS. 35-37). In all of the aforementioned applications, iStar was able to effectively characterize tissue architecture with high resolution. For example, iStar accurately detected TLSs that aligned well with the pathologist's manual annotation, as shown in FIG. 33.

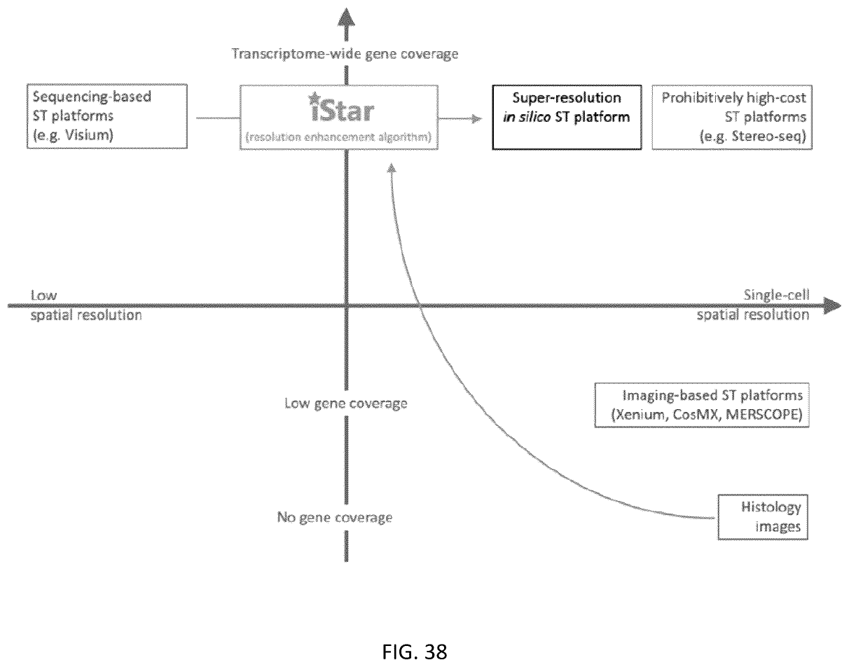

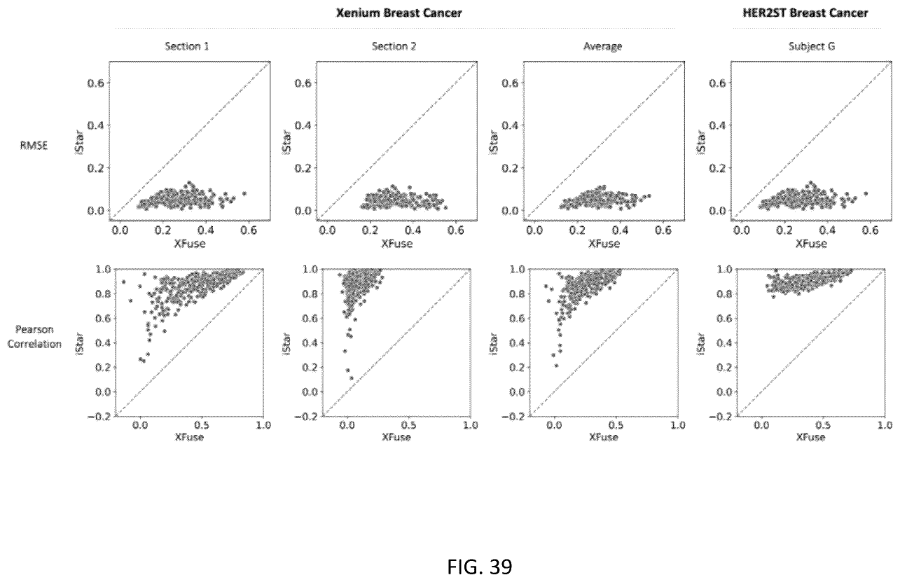

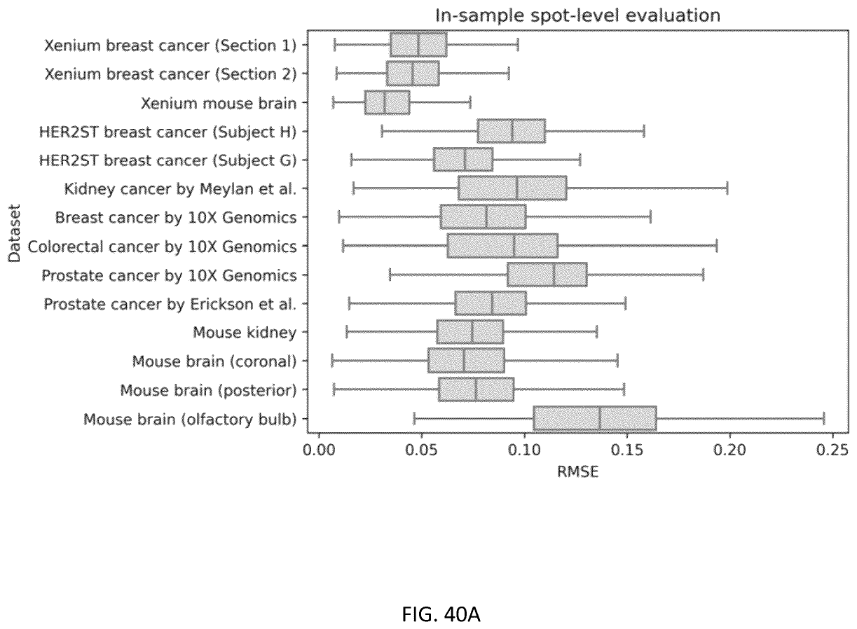

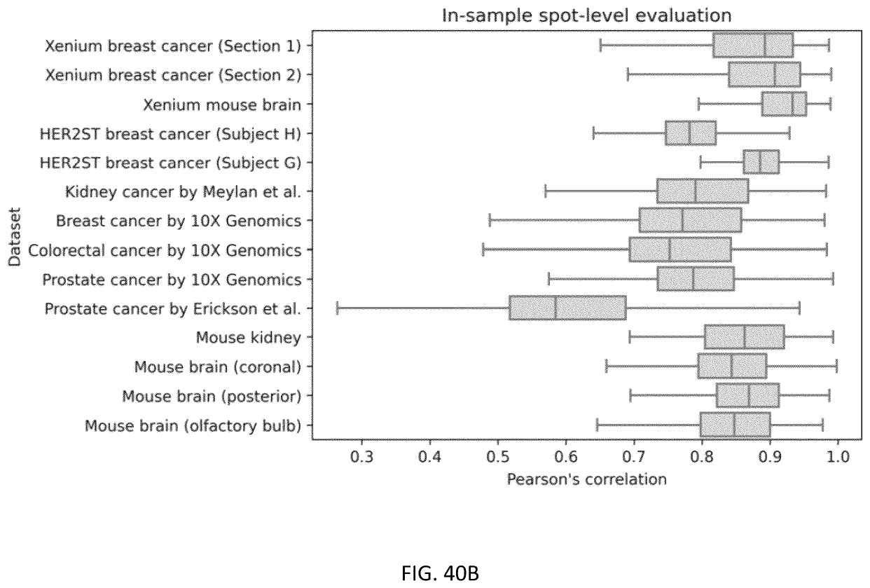

In summary, we have presented iStar, an end-to-end workflow for rapid annotation of super-resolution tissue architecture based on ST data generated from platforms that lack single-cell resolution. This holds significant implications for practical studies, as existing ST platforms lack either single-cell resolution or whole-transcriptome coverage. However, iStar allows us to generate ST data that cover the entire transcriptome with near-single-cell resolution, as shown in FIG. 38. A key step of iStar is to leverage the high-resolution histology image obtained from the same ST tissue section to reconstruct the unobserved super-resolution gene expression. Through the analysis of several datasets across multiple cancer types (e.g., breast, colorectal, prostate, and kidney) and healthy tissues (e.g., mouse kidney and mouse brain), we have demonstrated that the super-resolution gene expressions predicted by iStar are accurate. These predictions not only preserve the original gene expression at the spot-level, as shown in FIGS. 39 and 40, but also have practical applications in various tissue architecture inference tasks. Moreover, we have shown that iStar can perform out-of-sample prediction for tissue sections where only histology images are available. Furthermore, iStar is computationally efficient, with the end-to-end analysis of the Xenium-derived pseudo-Visium breast cancer data taking only 9 minutes, as shown in Table 1. By contrast, XFuse took 1,969 minutes (more than 32 hours) to analyze the same data. This advantage in computational efficiency allows iStar to generate virtual ST data from a large number of consecutively cut tissue sections with histology images, enabling a comprehensive characterization of gene expression variations in 3D tissues.

TABLE 1
Runtime comparison

| | | Runtime (minute) | | |
|---|---|---|---|---|
| Dataset | Method | Training | Prediction | Total |
| Xenium breast cancer | iStar | 8 | 1 | 9 |
| Xenium breast cancer | XFuse | 1946 | 23 | 1969 |
| Xenium mouse brain | iStar | 5 | 1 | 6 |
| Xenium mouse brain | XFuse | 1197 | 8 | 1205 |

TABLE 2
TLS marker genes.
TLS
markers
CD4
CD8A
CD74
CD79A
IL7R
ITGAE
CD1D
CD3D
CD3E
CD8B
CD19
CD22
CD52
CD79B
CR2
CXCL13
CXCR5
FCER2
MS4A1
PDCD1
PTGDS
TRBC2

### Methods

The Algorithm of iStar

The algorithm of iStar consists of three components: the histology feature extractor, super-resolution gene expression predictor, and tissue architecture annotator.

### Histology Feature Extractor

To facilitate the processing of histology images with different resolutions, we first rescale each image such that the size of one pixel is 0.5×0.5 μm². This rescaling ensures a 16×16-pixel tile corresponds to 8×8 μm², which is about the size of a single cell. To simplify the subsequent tiling procedure, we pad the rescaled image so that its height and width are both divisible by 256.

Next, we partition the whole image into image tiles hierarchically such that the large (high-level) tiles reflect the global tissue structure, whereas the small (low-level) tiles within a large tile reflect the local fine-grained cellular structure of the tissue. Let X∈ [symbol] ^{M}× [symbol] N× [symbol] ³ be the RGB-channel histology image with height M and width N. We first partition X into a (M/256)-row, (N/256)-column rectangular grid of 256×256-pixel image tiles: X=[Xₘ_{₁}ₙ_{₁}]ₘ_{₁}_{=1,n}_{₁}₌₁^{M/256,N/256}, where each Xₘ_{₁}ₙ_{₁}∈ [symbol] ²⁵⁶× [symbol] ²⁵⁶× [symbol] ³. Next, each 256×256-pixel image tile is further partitioned into a 16-row, 16-column rectangular grid of 16×16-pixel image tiles:

$${x}_{{m}_{1} {n}_{1}} = {[ {X}_{{m}_{1} {n}_{1} {m}_{2} {n}_{2}} ]}_{{m}_{2} = 1 , {n}_{2} = 1}^{16 , 16} , where each {X}_{{m}_{1} {n}_{1} {m}_{2} {n}_{2}} ∈ {ℝ}^{16} \times {ℝ}^{16} \times {ℝ}^{3} .$$

To extract hierarchical histology features, we use a hierarchical vision transformer architecture that consists of a local vision transformer (ViT) f₁°f₂ and a global ViT f₀. First, within each 256×256-pixel image tile, the local ViT maps each 16×16-pixel sub-tile into a low-level local feature vector of length C₂, that is, Zₘ_{₁}ₙ_{₁}ₘ_{₂}ₘ_{₂}ₙ_{₂}=f₂(Xₘ_{₁}ₙ_{₁}ₘ_{₂}ₙ_{₂})∈ [symbol] ^{C}^{₂}, and then maps all the 256 low-level local feature vectors within the 256×256-pixel image tile into a high-level local feature vector of length C₁, that is, Zₘ_{₁}ₙ_{₁}=f₁([Zₘ_{₁}ₙ_{₁}ₘ_{₂}ₙ_{₂}]ₘ_{₂}_{=1,n}_{₂}₌₁^{16,16})∈ [symbol] ^{C}₁.

Next, to model long-range dependencies of histology features within the whole image, the global ViT maps all the high-level local features within the whole image into high-level global features of the same dimension:

$${[ {t}_{{m}_{1} , {n}_{1}} ]}_{{m}_{1} = 1 , {n}_{1} = 1}^{M / 256 N / 256} = {f}_{0} ( {[ {z}_{{m}_{1} {n}_{1}} ]}_{{m}_{1} = 1 , {n}_{1} = 1}^{M / 2 \; 56 , N / 256} ) ∈ {ℝ}^{M / 256} \times {ℝ}^{N / 256} \times {ℝ}^{{C}_{1}} .$$

After this hierarchical histology feature extraction procedure, we have:
1. the high-level global feature image T=[tₘ_{₁}_{,n}_{₁}]ₘ_{₁}_{=1,n}_{₁}₌₁^{M/256,N/256}, which is an image of size (M/256)×(N/256) with C₁ channels,
2. the low-level local feature image Z=[Zₘ_{₁}_{=1,n}_{₁}_{=1,m}_{₂}_{=1,}_{ₙ}₂]ₘ_{₁}_{=1,n}_{₁}_{=1,m}_{₂}_{=1,n}_{₂}_{=1′}^{M/256,N/256}, which is an image of size (M/16)×(N/16) with C₂ channels, and
3. the original RGB image, which is an image of size M×N with 3 channels.
   To align these feature images, we use bicubic interpolation to resize each image into the desired size M′×N′ and stack the channels of the resized images, which results in a combined histology feature image H=[hₘₙ]_{m=1,n=1}^{M′,N′} of size M′×N′ with C₁+C₂+3 channels, where each hₘₙ∈ [symbol] ^{C}^{₁}^{+C}^{₂}⁺³ is the histology feature vector at pixel (m, n). In our implementation, we set C₁=192 and C₂=384. For the image size, we varied (M′,N′) among (M/16,N/16), (M/32,N/32), (M/64,N/64), and (M/128,N/128).

To train the histology feature extractor, we optimize the ViTs through self-supervised learning (SSL). Because of the benefits of transfer learning on ViTs, the model is pretrained on publicly available histology datasets. In this step, since only histology images are needed and no gene expression data or image-level labels are required, many publicly available histology datasets, such as the Cancer Genome Atlas (TCGA), the Genotype-Tissue Expression (GTEx) project, and the kidney biopsies in Holscher et al. (2023), are suitable for pretraining the model. Moreover, for the choice of the SSL algorithm, any SSL method for analyzing image data, such as DINO or BEIT, is suitable for our purpose. In our implementation, we adopted the pretrained model in Chen et al. (2022), which uses DINO to train the ViTs hierarchically on the TCGA data. In our experiments, we found that the pretrained model was able to capture the histology characteristics well and thus decided to skip the fine-tuning step to improve computation efficiency.

### Super-Resolution Gene Expression Predictor

Once the histology feature images have been extracted, we use them to predict super-resolution gene expression. The histology features at every superpixel contain information on not only its local cellular characteristics but also its global relationships to other regions in the whole image. Thus the gene expression predictor does not need to explicitly model the spatial dependencies, since correlation between superpixels are already reflected in the similarity between their high-level global histology features (i.e. the C₁ channels in the combined histology feature image H), even if the superpixels are physically far away from each other. Therefore, when predicting the gene expression at a superpixel, the input of the predictor only includes the histology features at this superpixel, and no convolution, attention, or any other mechanisms with spatial awareness are needed, which significantly reduces the computation cost.

To train the super-resolution gene expression predictor, since the model output is at the superpixel level but the training data are at the spot level, we adopt a weakly supervised learning framework. We model the gene expression observed at each spot as the sum of the superpixels' gene expression inside that spot. This model design mimics the data collection procedure of sequencing-based ST platforms, which barcodes and combines all the transcripts inside a spot into a sample and sends it for next-generation sequencing. To express the loss function, let S be the number of spots in the whole image, K be the number of genes to predict, gₖ be the gene expression prediction model for gene k, yₖₛ be the observed gene expression for gene k at spot s, Mₛ be the collection of superpixels covered by spot s, and hₘₙ be the histology feature vector at superpixel (m, n). Then the weakly supervised loss function is

$$ℒ = {\sum }_{k = 1}^{K} {\sum }_{s = 1}^{S} {( {y}_{k s} - {\sum }_{( m , n ) ∈ {M}_{s}} {g}_{k} ( {h}_{m n} ) )}^{2} .$$

Superpixels outside the spot masks, including the between-spot gaps and the background image, are excluded during model training. After model training, the predicted gene expression for gene k at superpixel (m, n) is ŷₖₘₙ=gₖ(hₘₙ), which gives us the gene expression image Ŷₖ=[ŷₖₘₙ]_{m=1,n=1}^{M′,N′}. Furthermore, if cell segmentation masks are provided, single cell-level gene expression can be obtained using the predicted superpixel-level gene expressions, where the former is computed as a weighted sum of the latter, with the weight equal to the proportion of the superpixel that overlaps with the cell mask. In our experiments, we only predicted the union of the top 1,000 most highly variable genes in each dataset and the marker genes for the user-defined structures (e.g. TLS), since lowly variables genes had low signal-to-noise ratios and would introduce extra noise to the model training procedure. The only two exceptions are the benchmark experiments using the Xenium breast cancer dataset (313 genes) and the Xenium mouse brain dataset (248 genes), in which case we predicted all the genes due to their small number and the need for method evaluation.

For network architecture of the gene expression prediction model, we use a feed-forward neural network with 4 hidden layers and 256 nodes per hidden layer. The leaky rectified linear unit (ReLU) is used as the activation function for the hidden layers. The output layer is a linear layer with 256 input nodes and K output nodes, and the outputs are activated by an exponential linear unit (ELU) to ensure that the predicted gene expressions are non-negative.

### Tissue Architecture Annotator

Once obtaining the super-resolution gene expression, we segment the tissue by clustering the superpixels using their gene expression information. First, we obtain gene expression embeddings by reducing the dimension of the predicted gene expression vector, where each superpixel is treated as a sample and each gene as a feature. Although any dimension reduction technique (e.g. PCA or UMAP) can obtain gene expression embeddings from the predicted super-resolution gene expression, we recommend treating the intermediate values in the second-last feed-forward layer of the gene expression prediction model as the gene expression embeddings, since they are not only low-dimensional (256 in our setting) but also linearly related to the predicted gene expression vectors, and using these pre-computed values does not incur any additional computational cost. Next, to promote spatial contiguousness in segmentation, we smooth the gene expression embeddings by a Gaussian filter, an approach that is similar in spirit to the sliding-window method for cell neighborhood identification. Then we treat the smoothed gene expression embedding vector at every superpixel as a sample and cluster all the superpixels with the k-means algorithm. This procedure partitions the tissue into functionally distinct regions in an unsupervised manner based on their gene expression profiles.

To assign biologically meaningful interpretations to the tissue regions in the segmentation, we perform cell type inference at the superpixel level, where we treat each superpixel as an artificial cell and infer its cell type using its predicted gene expressions along with a marker gene reference panel. Recall that the total number of genes in the model is [symbol] . Let T be the total number of candidate cell types. For each cell type t∈{1, . . . , T}, suppose we have a list of marker gene indices Aₜ, which is a subset of {1, . . . , [symbol] }. For example, in our experiments with breast cancer data, we used the marker gene lists provided by Wu et al. For each marker gene k∈Aₜ, we standardize its predicted super-resolution gene expression image [symbol] =[ŷₖₘₙ]_{m=1,n=1}^{M′,N′}∈ [symbol] ^{M′N′} into the range of [0.0,1.0] and obtain \tilde{Y}ₖ=[\tilde{y}ₖₘₙ]_{m=1,n=1}^{M′N′}∈ [symbol] ^{M′,N′}, where \tilde{y}ₖₘₙ=(ŷₖₘₙ−min Ŷₖ). Then for each superpixel (m,n), we compute the score for cell type t by averaging the standardized gene expressions of all its marker genes: uₜₘₙ=|Aₜ|⁻¹Σ_{k∈A}_{ₜ}\tilde{y}ₖₘₙ where |Aₜ| is the number of genes in Aₜ. To infer the cell type of superpixel (m,n), let

$${t}_{m n}^{max} = {arg max}_{1 \leq t \leq T} {u}_{tmn}$$

be the cell type with the maximal score and

$${u}_{m n}^{max} = {max}_{1 \leq t \leq T} {u}_{tmn}$$

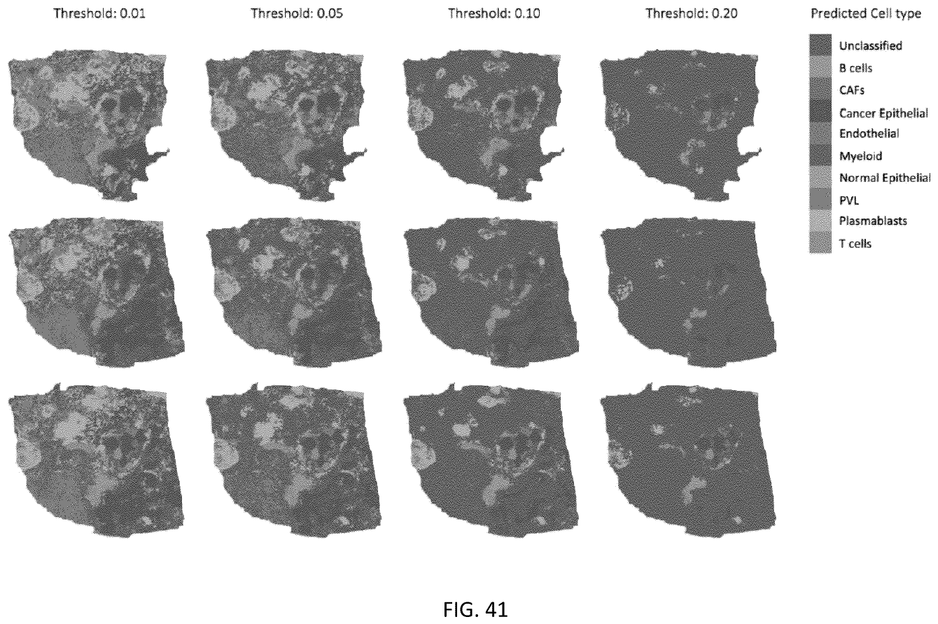

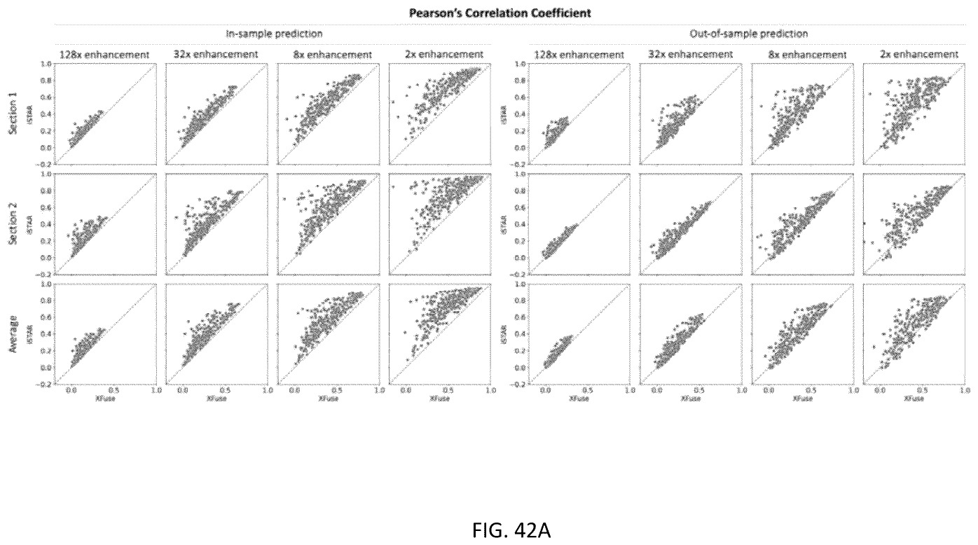

be the score of this cell type. Given a predetermined threshold u_{threshold}∈[0,1] if uₘₙ^{max}>u_{threshold}, then the cell type of superpixel (m,n) is predicted to be tₘₙ^{max}; otherwise, the cell type of this superpixel is unclassified. In our experiments, we set u_{threshold}=0.1 and found it effective in most cases. See FIG. 41 for a demonstration of the effects of u_{threshold} on cell type inference. While this score-based approach was used for cell type inference in our experiments, any cell type annotation tool serves the purpose. For example, when a well-annotated single-cell RNA-seq reference panel is available, the cell types can be annotated by methods such as SingleR or ItClust. Finally, to combine the superpixel-level predicted cell types with the tissue clusters obtained through unsupervised segmentation, an enrichment analysis is applied to every cell type-tissue cluster pair, which elucidates the biological activities inside each cluster by examining the cell types over-represented in the cluster.

In addition to the above-described unsupervised tissue annotation procedure, iStar also allows annotation with user-defined tissue structures. Given a user-defined list [symbol] of marker genes for the structure of interest (e.g. TLS, Table 2), for every gene k in [symbol] , we first standardize its predicted gene expression image [symbol] =[ŷₖₘₙ]_{m=1,n=1}^{M′,N′}∈ [symbol] ^{M′,N′}; into the range of [0.0, 1.0] and obtain the standardized image \tilde{Y}ₖ∈ [symbol] ^{M′N′}. Then we compute the score for the user defined tissue structure by averaging the standardized gene expression of all the user-define marker genes: [symbol] =| [symbol] |⁻¹ [symbol] \tilde{Y}ₖ, where | [symbol] | is the number of genes in [symbol] . The resulting score image U [symbol] reflects the activity of the user-defined structure in the tissue.

### Benchmark Data Generation

To evaluate the super-resolution gene expression prediction accuracy, we generated spot-level pseudo-Visium data using pixel-level Xenium data. The pixel size of the Xenium gene expression images was 0.2×0.2 μm², and we rescaled the pixel size to 0.5×0.5 μm². The gene expression measurements in Xenium were binned into spots based on the spot size, shape and layout of Visium: a hexagonal grid of disc-shaped spots with a spot diameter of 55 μm and a center-to-center distance of 100 μm. For the ground truth, we binned the Xenium gene expressions into a rectangular grid of superpixels, where the size of the superpixels varied among 8×8 μm², 16×16 μm², 32×32 μm², and 64×64 μm², depending on the experimental settings.

### Evaluation Criteria for Super-Resolution Gene Expression Prediction Accuracy

To evaluate the accuracy of the predicted super-resolution gene expressions, for each gene, we treated both the ground truth and the predicted gene expression as images, where the image intensity was standardized into the range of [0.0, 1.0]. Then the prediction accuracy was measured by the root mean squared error (RMSE) and the structural similarity index measure (SSIM). To compute the RMSE, the ground truth and the predicted gene expression images were flattened into vectors, and the RMSE was equal to the Euclidean distance between the two vectors. The RMSE is a straightforward and fast metric for assessing the prediction accuracy of any outcomes that can be vectorized, but for image data, the RMSE ignores the spatial contexts within the images. Thus, in addition to the RMSE, we also computed the SSIM to evaluate the similarity between the spatial structures of the ground truth and the predicted gene expression images. The SSIM is an image similarity metric that is widely used for super-resolution tasks in computer vision and medical imaging. A higher SSIM indicates a higher degree of similarity between two images. In our context, the SSIM captures both global trends and the fine-grained spatial structures in the super-resolution gene expression images. Our experiments showed that iStar significantly outperformed XFuse as measured by both the RMSE and SSIM.

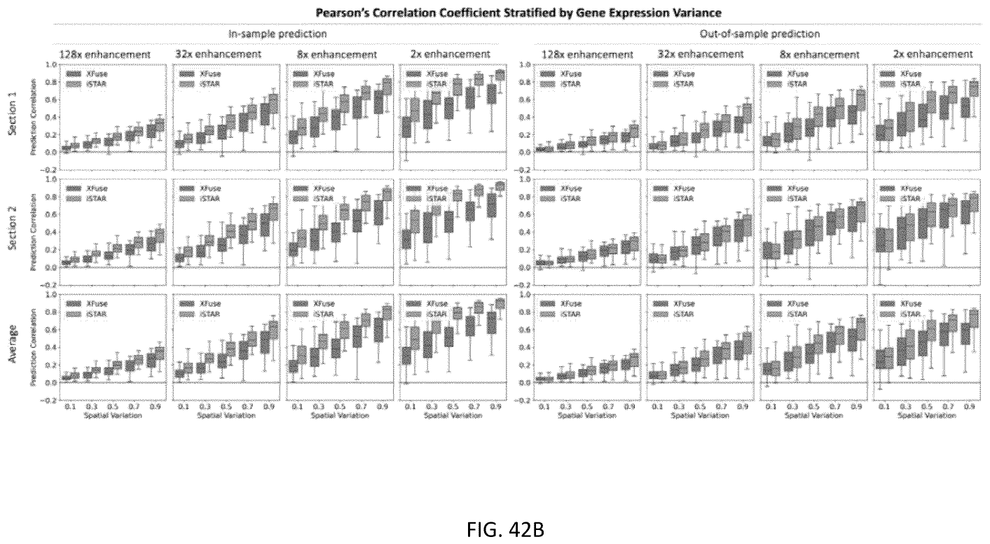

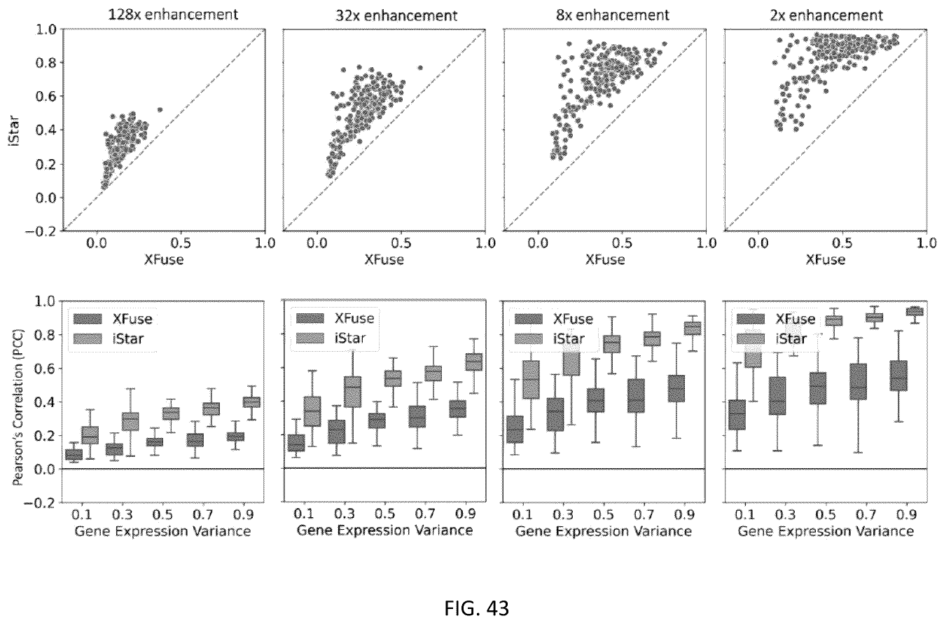

In addition to RMSE and SSIM, Pearson correlation coefficient (PCC), which is an uncommon metric for super-resolution tasks, was employed in some previous works on spatial transcriptomics as an evaluation criterion for gene expression prediction accuracy. However, these works studied spatial transcriptomics at low resolutions, where the number of spatial units (i.e. superpixels or spots) was no more than 2000, and the size of the spatial units was around 100 μm. By contrast, the prediction accuracy in our experiments was evaluated at a much higher resolution, where the number of superpixels was as large as 10⁶, and their size was as small as 8 μm. Due to the high image resolution and high noise magnitude in the ground truth, PCC is sensitive to outlying noisy superpixels, especially for sparsely expressed genes. In our experiments, compared to RMSE and SSIM, PCC had difficulties in differentiating superior and inferior super-resolution predictions when the resolution was high. As the resolution decreased, the noise level in the ground truth also decreased, which led to sharpened contrast between the accuracy of iStar and XFuse as measured by PCC. Furthermore, more spatially variables genes, which were associated with higher signal-to-noise ratio, produced higher PCCs and greater differences in PCC between iStar and XFuse, which again indicates the sensitivity of PCC to the noise level in the ground truth. Overall, PCC had limited power in differentiating super-resolution prediction accuracy for high-resolution, high-noise gene expression images. On the other hand, when the resolution and noise level were low, PCC produced results similar to those by RMSE and SSIM, as shown in FIGS. 42 and 43.

FIGS. 4A and 4B show predicted super-resolution gene expressions by iStar and XFuse compared to ground truth and spot-level gene expressions for genes in Section 1 tissue and Section 2 tissue, respectively, whose variances are in the 80%-100% qualities among all the 313 genes in the Xenium-derived pseudo-Visium data obtained from a breast cancer patient. The variance quantiles of the genes from left to right are 97.5%, 95.0%, . . . , 82.5%, and 80.0%, respectively. Super-resolution gene expressions were visualized at the scale of 8× resolution enhancement.

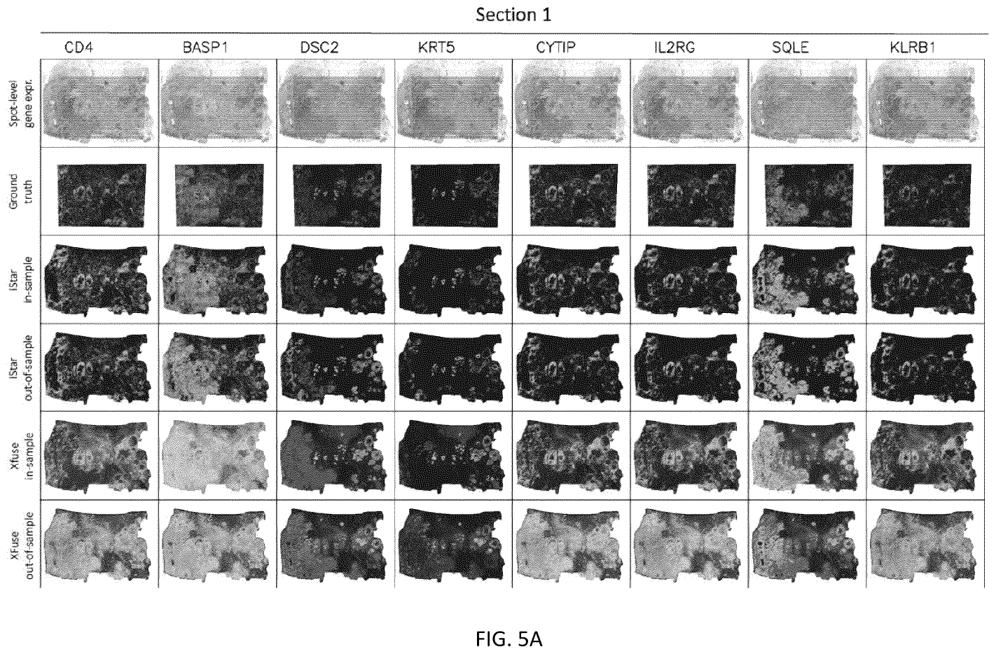

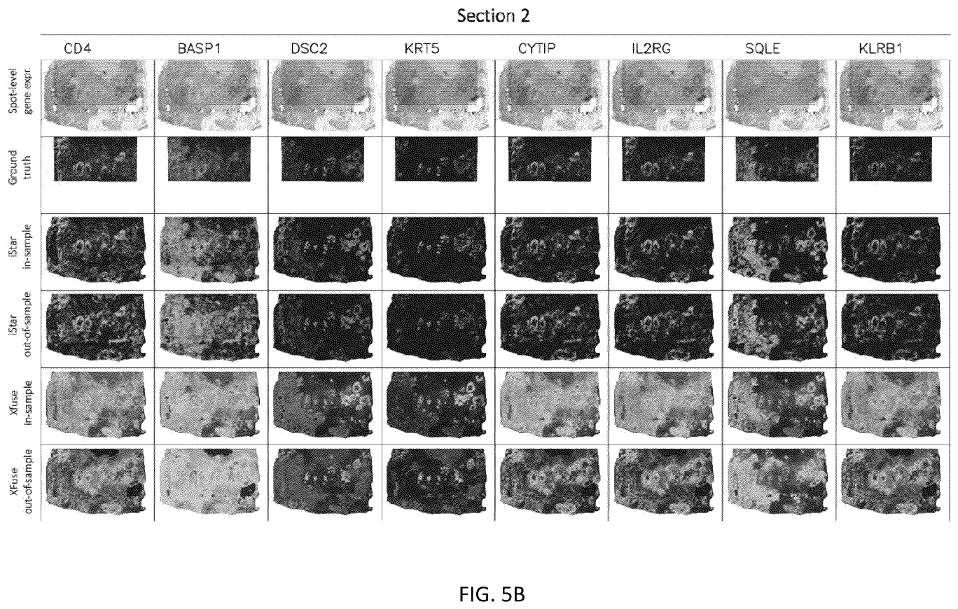

FIGS. 5A and 5B show predicted super-resolution gene expressions by iStar and XFuse compared to ground truth and spot-level gene expressions for genes in Section 1 tissue and Section 2 tissue, respectively, whose variances are in the 60%-80% qualities among all the 313 genes in the Xenium-derived pseudo-Visium data obtained from a breast cancer patient. The variance quantiles of the genes from left to right are 77.5%, 75.0%, . . . , 62.5%, and 60.0%, respectively. Super-resolution gene expressions were visualized at the scale of 8× resolution enhancement.

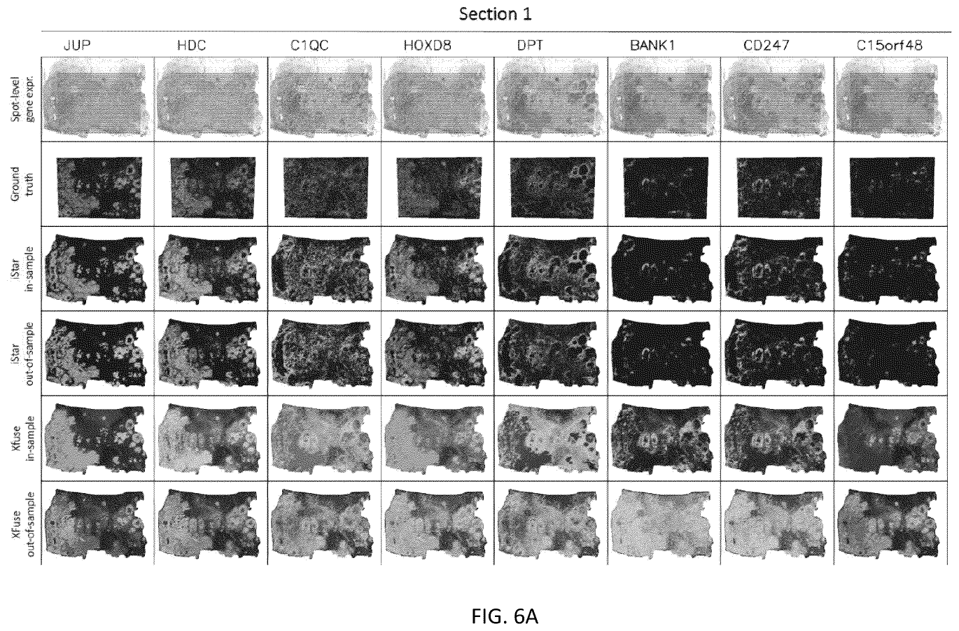

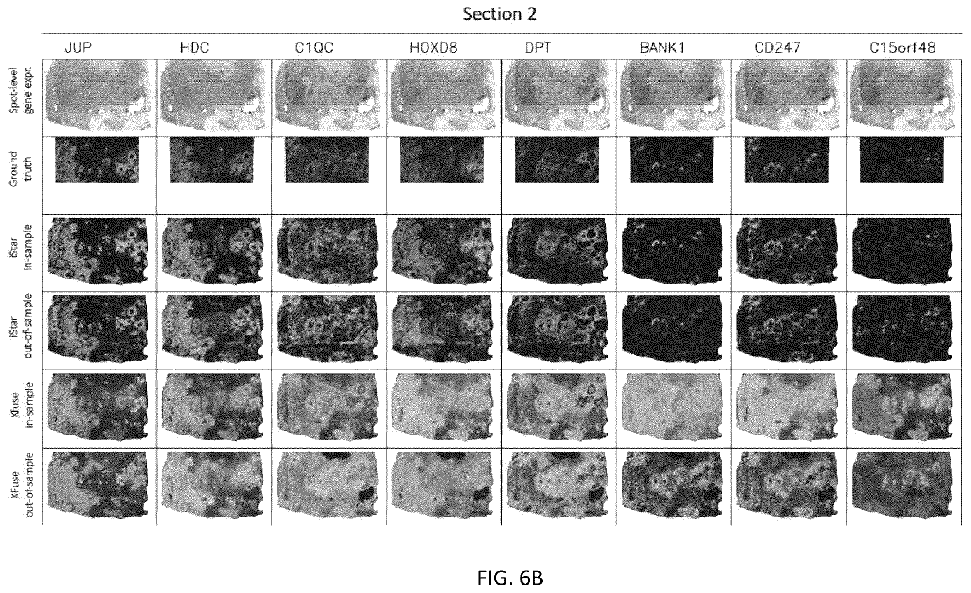

FIGS. 6A and 6B show predicted super-resolution gene expressions by iStar and XFuse compared to ground truth and spot-level gene expressions for genes in Section 1 tissue and Section 2 tissue, respectively, whose variances are in the 40%-60% qualities among all the 313 genes in the Xenium-derived pseudo-Visium data obtained from a breast cancer patient. The variance quantiles of the genes from left to right are 57.5%, 55.0%, . . . , 42.5%, and 40.0%, respectively. Super-resolution gene expressions are visualized at the scale of 8× resolution enhancement.

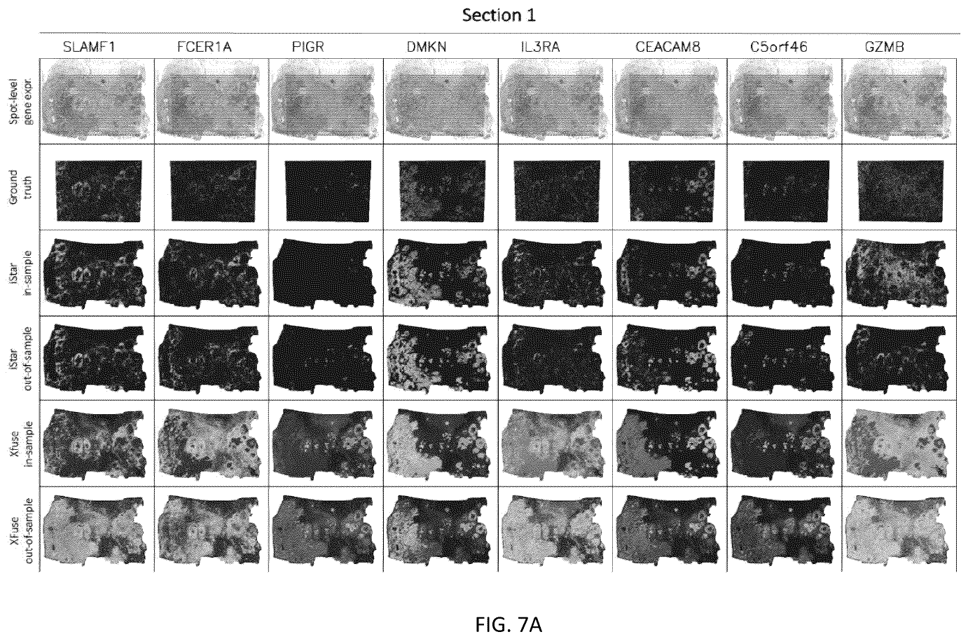

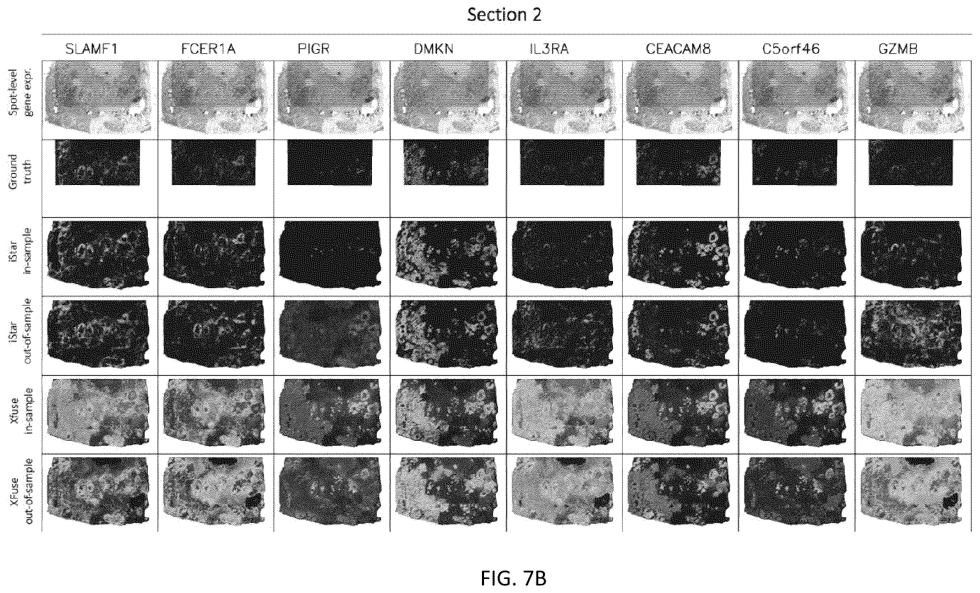

FIGS. 7A and 7B show predicted super-resolution gene expressions by iStar and XFuse compared to ground truth and spot-level gene expressions for genes in Section 1 tissue and Section 2 tissue, respectively, whose variances are in the 20%-40% qualities among all the 313 genes in the Xenium-derived pseudo-Visium data obtained from a breast cancer patient. The variance quantiles of the genes from left to right are 37.5%, 35.0%, . . . , 22.5%, and 20.0%, respectively. Super-resolution gene expressions are visualized at the scale of 8× resolution enhancement.

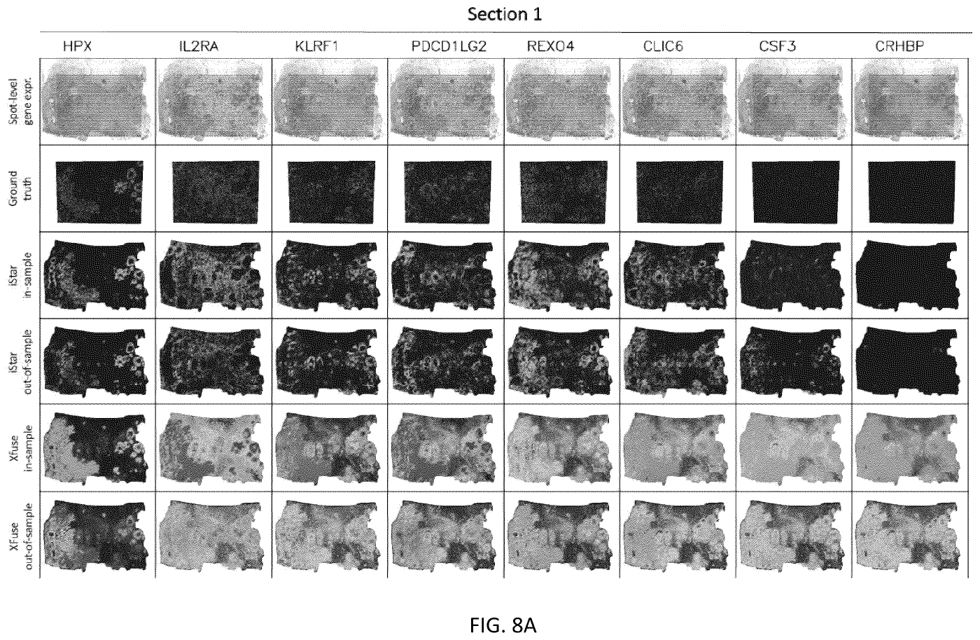

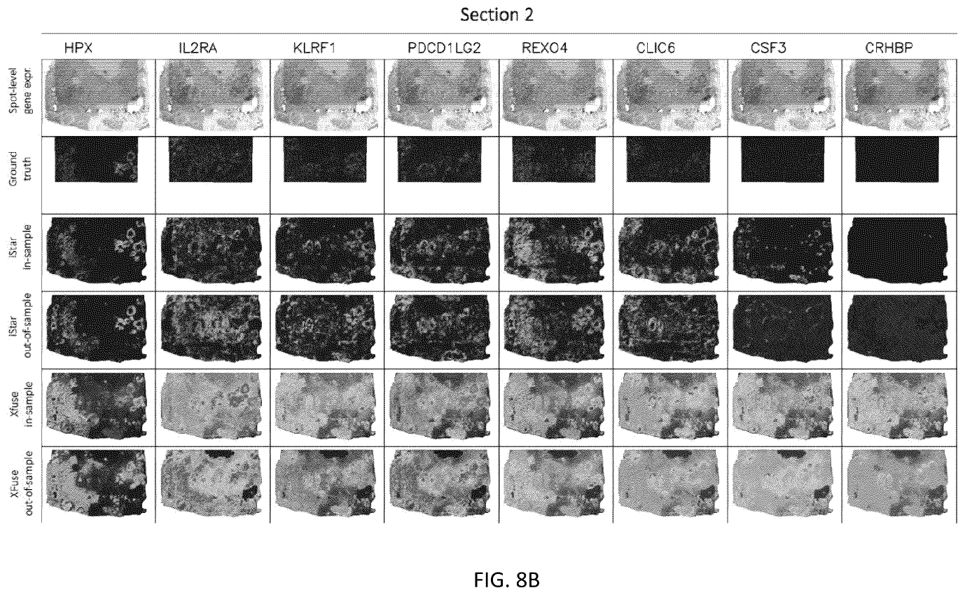

FIGS. 8A and 8B show predicted super-resolution gene expressions by iStar and XFuse compared to ground truth and spot-level gene expressions for genes in Section 1 tissue and Section 2 tissue, respectively, whose variances are in the 0%-20% qualities among all the 313 genes in the Xenium-derived pseudo-Visium data obtained from a breast cancer patient. The variance quantiles of the genes from left to right are 17.5%, 15.0%, . . . , 2.5%, and 0.0%, respectively. Super-resolution gene expressions are visualized at the scale of 8× resolution enhancement.

FIG. 9 shows predicted super-resolution gene expressions by iStar for three breast cancer-related genes (ESR1, ERBB2, and PGR) at various resolution enhancements;

FIGS. 10A and 10B show prediction accuracy of iStar and XFuse as measured by root squared error (RMSE) and structural similarity index measure (SSIM), respectively, for all the 313 genes in the Xenium-derived pseudo-Visium data obtained from a breast cancer patient. In each scatter plot, a dot represents a gene. In this analysis, Section 1 was first treated as the training sample in which in-sample prediction was performed for Section 1 and then out-of-sample prediction was performed for Section 2. We then repeated this analysis by treating Section 2 as the “in-sample” and Section 1 as the “out-of-sample”. The evaluation metrics in “Average” is the average of those in Sections 1 and Section 2.

FIG. 11 compares iStar predicted super-resolution gene expression patterns with corresponding histology image features. iStar predicted super-resolution gene expression patterns correlate well with the underlying histology image features.

FIG. 12 shows single-cell level gene expression predicted by iStar pseudo-Visium breast cancer data derived from Xenium data. Shown on the left is the ground truth single-cell level gene expression directly measured by Xenium, and shown on the right is the single-cell level gene expression predicted by iStar.

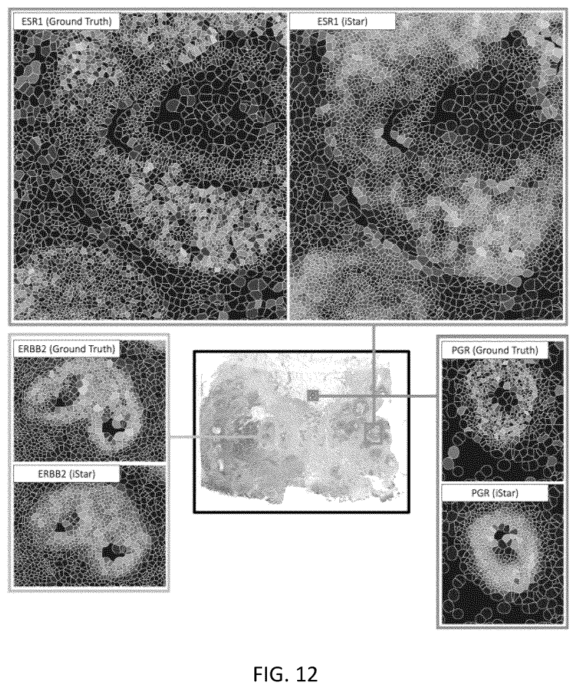

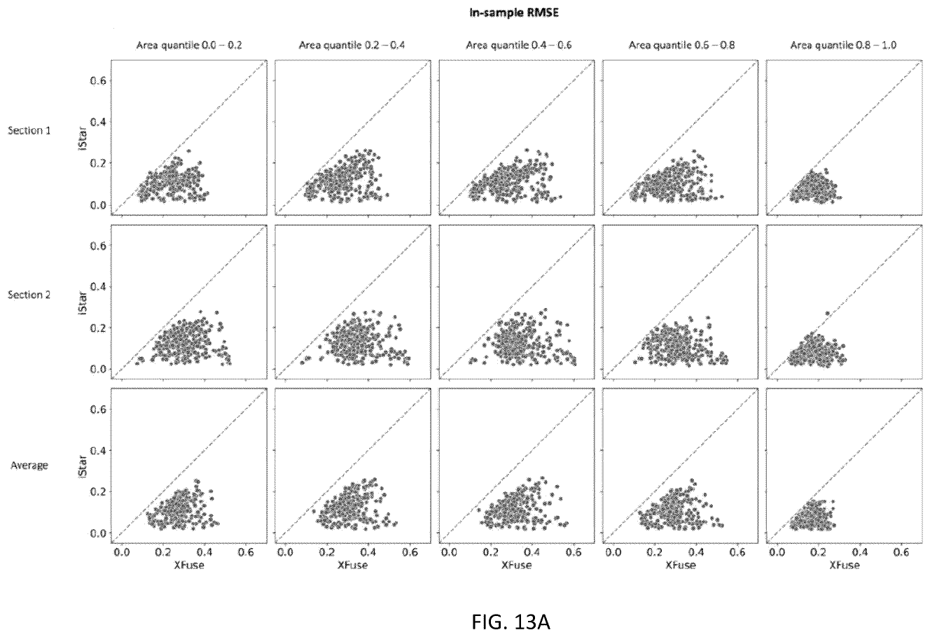

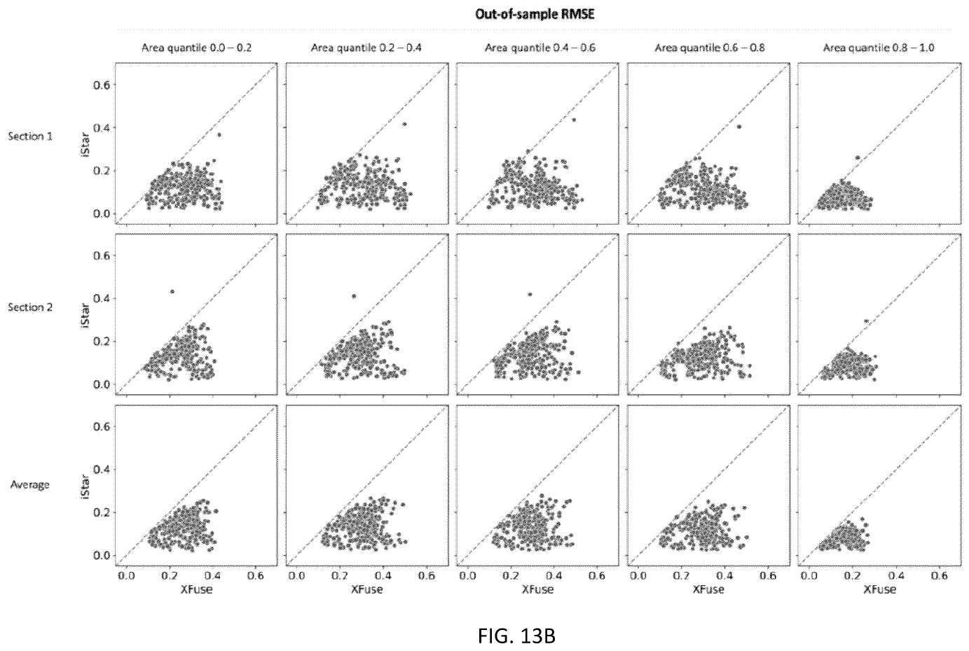

FIGS. 13A and 13B show accuracy of iStar and XFuse for single-cell level gene expression in-sample prediction for Section 1 out-of-sample prediction for Section 2, respectively, as measured by RMSE for all the 313 genes in the Xenium-derived pseudo-Visium data obtained from a breast cancer patient. In each scatter plot, a dot represents a gene. In this analysis, Section 1 was first treated as the training sample in which in-sample prediction was performed for Section 1 and then out-of-sample prediction was performed for Section 2. We then repeated this analysis by treating Section 2 as the “in-sample” and Section 1 as the “out-of-sample”. The evaluation metrics in “Average” is the average of those in Sections 1 and Section 2. We stratified cells by the quantiles of their cell size.

FIG. 14 compares tissue segmentation results by iStar and XFuse on a Xenium-derived pseudo-Visium breast cancer dataset.

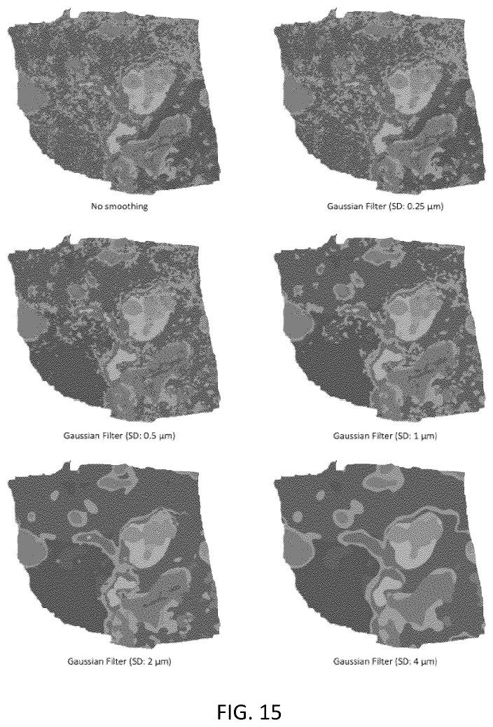

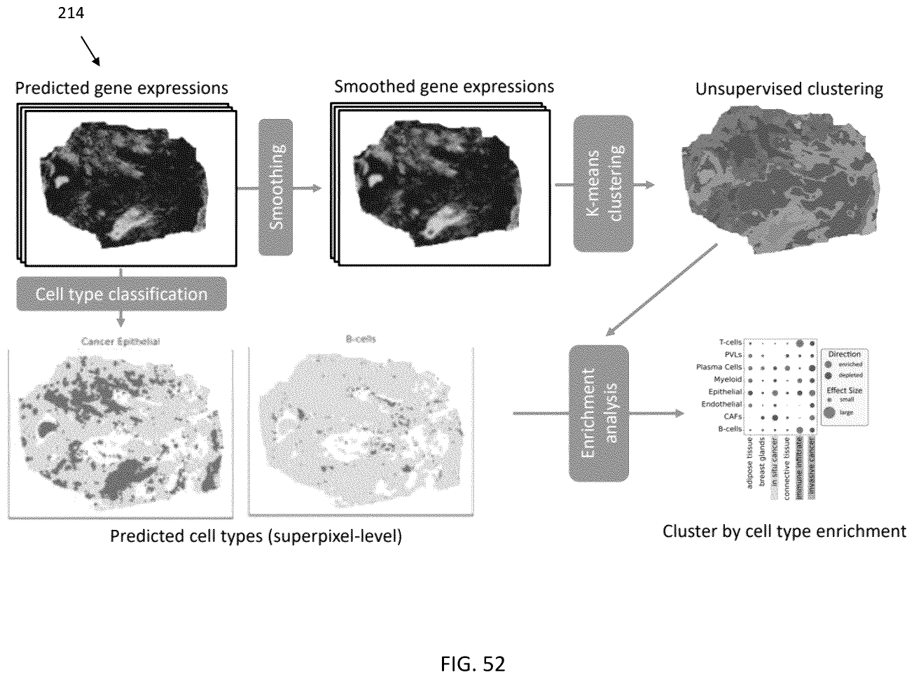

FIG. 15 shows iStar segmentation with different strengths of smoothing in a Gaussian filter in sample H1 of the HER2ST breast cancer dataset. In this analysis, the Gaussian filter smoothed gene expression embeddings obtained from the second last layer of the feed-forward neural network in the gene expression predictor. The smoothed embeddings were then used as inputs for K-means clustering, as also shown in FIG. 52. The standard deviation (SD) in the Gaussian filter determines the degree of smoothing.

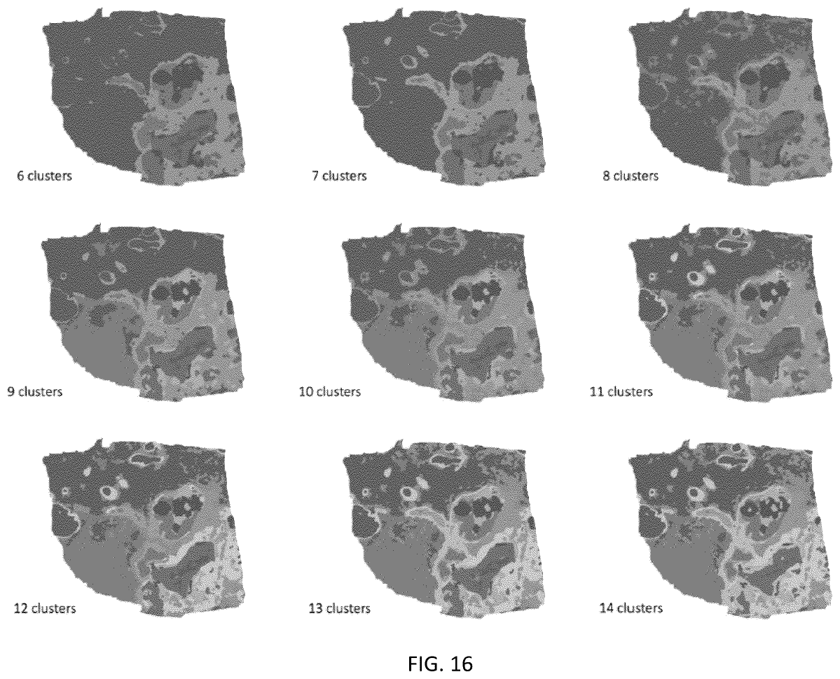

FIG. 16 shows iStar segmentation with different numbers of clusters in sample H1 of the HER2ST breast cancer dataset.

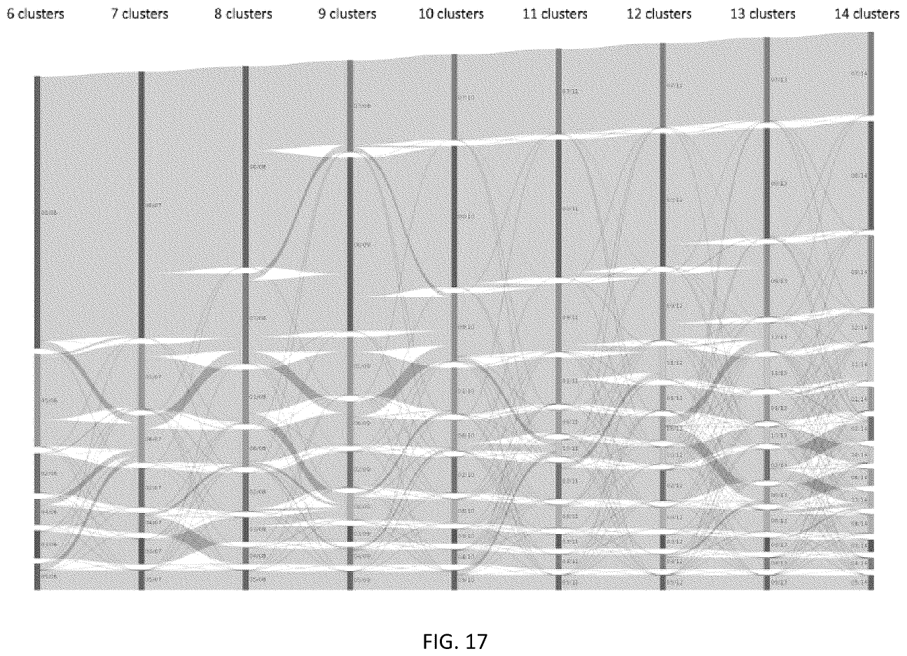

FIG. 17 is a river plot for iStar segmentation with different numbers of clusters in sample H1 of the HER2ST breast cancer dataset.

FIGS. 18A-18C shows Gene expression score for each cell type and the predicted cell type map in the three consecutively cut tissue sections of Subject H in the Anderson et al. HER2ST breast cancer dataset. FIG. 18A shows gene expression score for cell types B cells, CAFs, and cancer epithelial. FIG. 18B shows gene expression score for cell types endothelial, myeloid, and normal epithelial. FIG. 18C shows gene expression score for cell types plasmablasts, PLV, and T cells. Super-resolution was performed with 128× resolution enhancement.

FIGS. 19A and 19B show the most over-expressed gene in each automatically detected tissue cluster in three consecutively cut tissue sections of Subject H in the Anderson et al. HER2ST breast cancer dataset. Only clusters that have at least one gene with a mean fold change of 2.0 or above were included. Super-resolution was performed with 128× resolution enhancement.

FIGS. 20A-20B show TLS score and the super-resolution gene expression of seven TLS marker genes in the three consecutively cut tissue sections of Subject G in the Anderson et al. HER2ST breast cancer dataset. FIG. 20A shows the TLS score and the super-resolution gene expression of TLS marker genes MSA1, CD3D, and CR2. FIG. 20B shows the super-resolution gene expression of CXCR5, CXCL13, CD4, and CD8A. Super-resolution was performed with 128× resolution enhancement.

FIGS. 21A-21B show TLSs of Subject H and Subject G, respectively, in the Anderson et al. HER2+ breast cancer dataset detected by iStar. Displayed are the predicted TLS scores by iStar and the original publication, along with the pathologist's manual annotation reported in the original publication. Super-resolution was performed with 128× resolution enhancement.

FIGS. 22-22D show analysis of a breast cancer Visium dataset generated by 10× Genomics. The tissue was AJCC/UICC Stage T2N0M0, ER positive, PR negative, and HER2 positive. iStar was applied to this dataset in which the top 1,000 highly variable genes together with additional T cell marker genes in Wu et al. (with a total of 1,194 genes) were included in this analysis. FIG. 22A shows a pathologist annotation of the breast cancer dataset. FIG. 22B shows a total gene expression for each spot in the breast cancer dataset. FIG. 22C shows an iStar segmentation of the breast cancer dataset. FIG. 22D shows an iStar cell type annotation overtop histology of the breast cancer dataset.

FIGS. 23A-23C show spot-level training data, ground truth gene expression, and predicted super-resolution gene expression by iStar and xFuse for 24 genes whose variance are in the 80%-100% qualities among all the 248 genes in the Xenium-derived pseudo-Visium data obtained from mouse brain. The variance quantiles of the genes in the order of top-left, top-right, bottom-left, bottom-right are equally spaced from 100% to 80% (in descending order). Super-resolution gene expressions are visualized at the scale of 8× resolution enhancement.

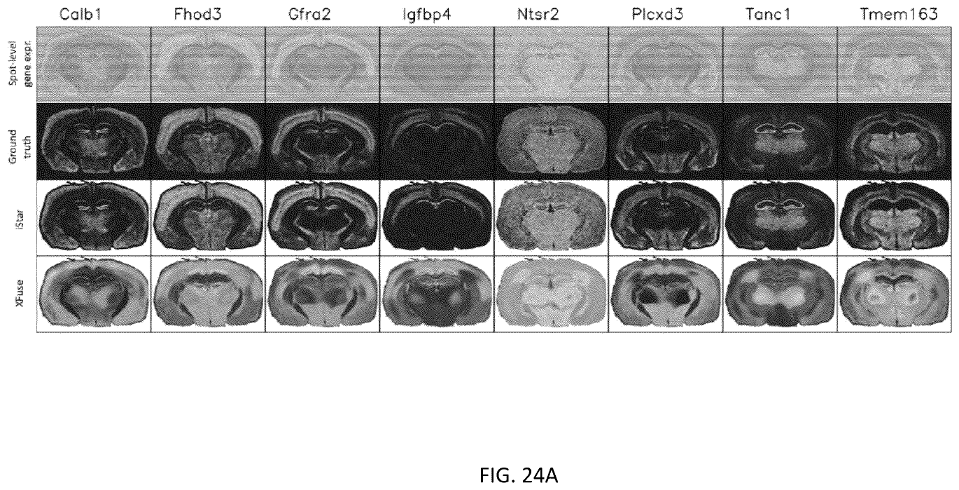

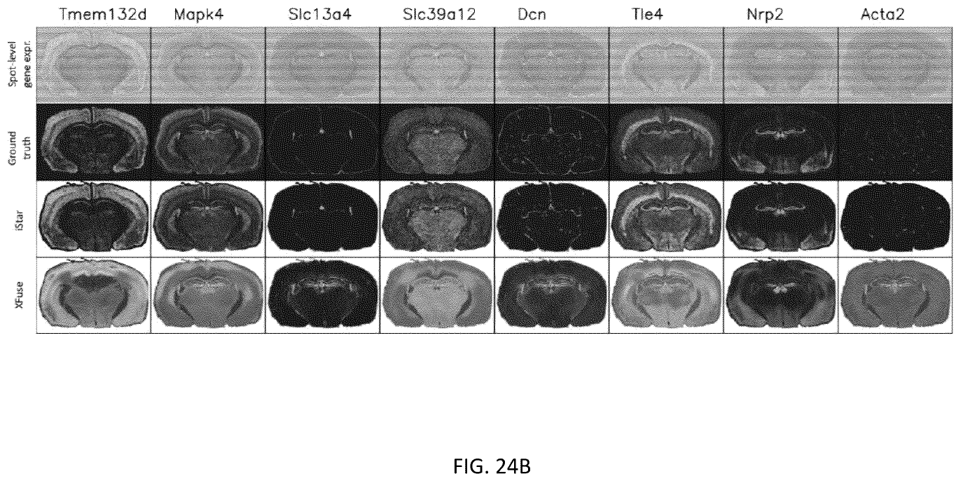

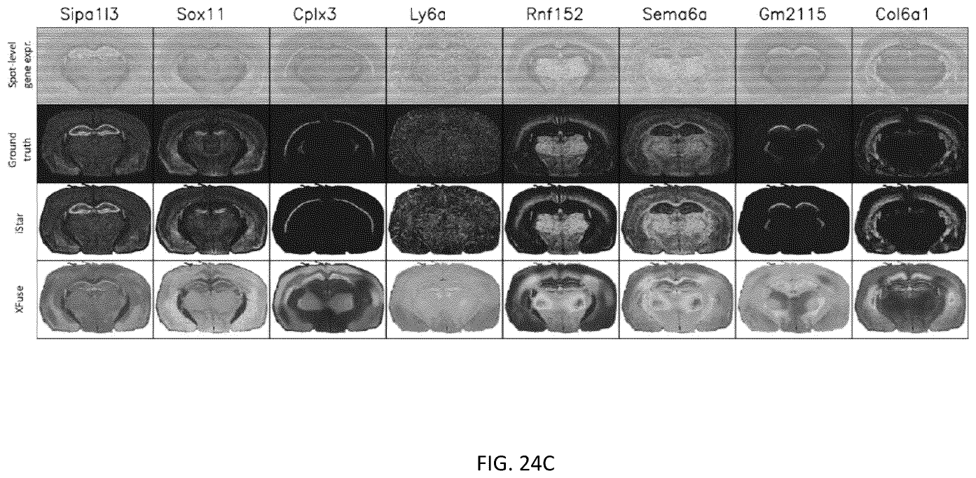

FIGS. 24A-24C show spot-level training data, ground truth gene expression, and predicted super-resolution gene expression by iStar and xFuse for 24 genes whose variance are in the 60%-80% qualities among all the 248 genes in the Xenium-derived pseudo-Visium data obtained from mouse brain. The variance quantiles of the genes in the order of top-left, top-right, bottom-left, bottom-right are equally spaced from 80% to 60% (in descending order). Super-resolution gene expressions are visualized at the scale of 8× resolution enhancement.

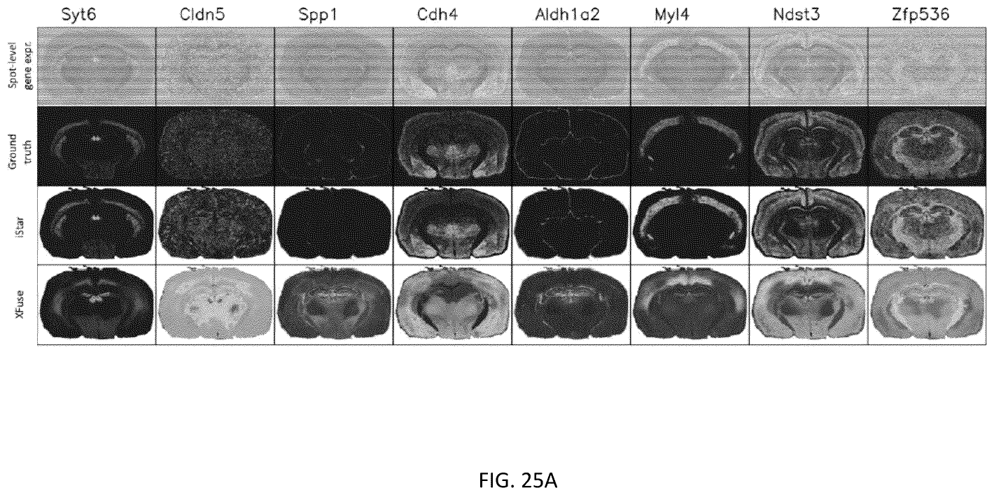

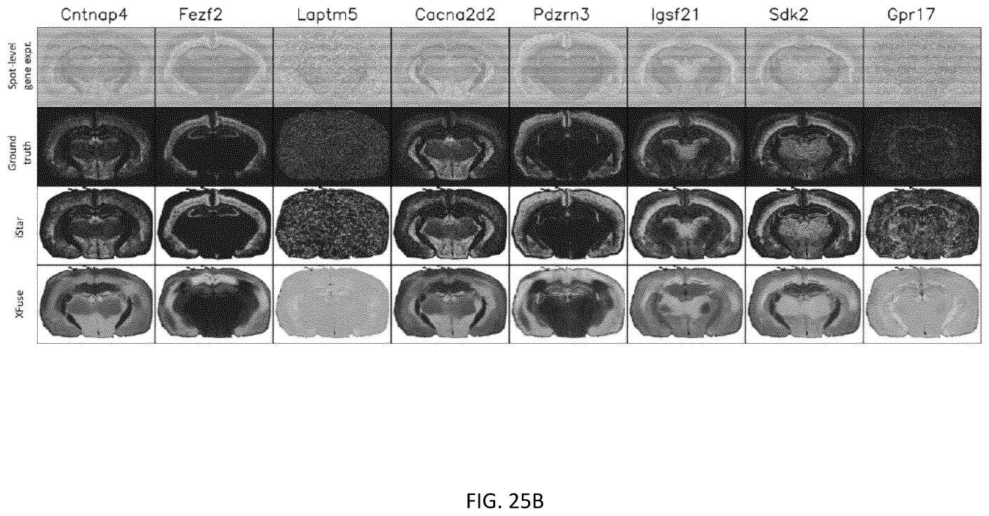

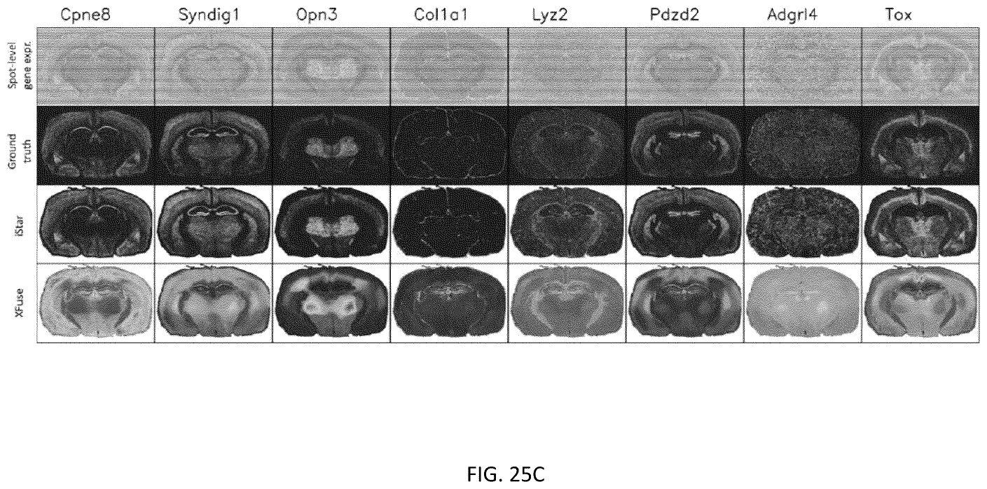

FIGS. 25A-25C show spot-level training data, ground truth gene expression, and predicted super-resolution gene expression by iStar and xFuse for 24 genes whose variance are in the 40%-60% qualities among all the 248 genes in the Xenium-derived pseudo-Visium data obtained from mouse brain. The variance quantiles of the genes in the order of top-left, top-right, bottom-left, bottom-right are equally spaced from 60% to 40% (in descending order). Super-resolution gene expressions are visualized at the scale of 8× resolution enhancement.

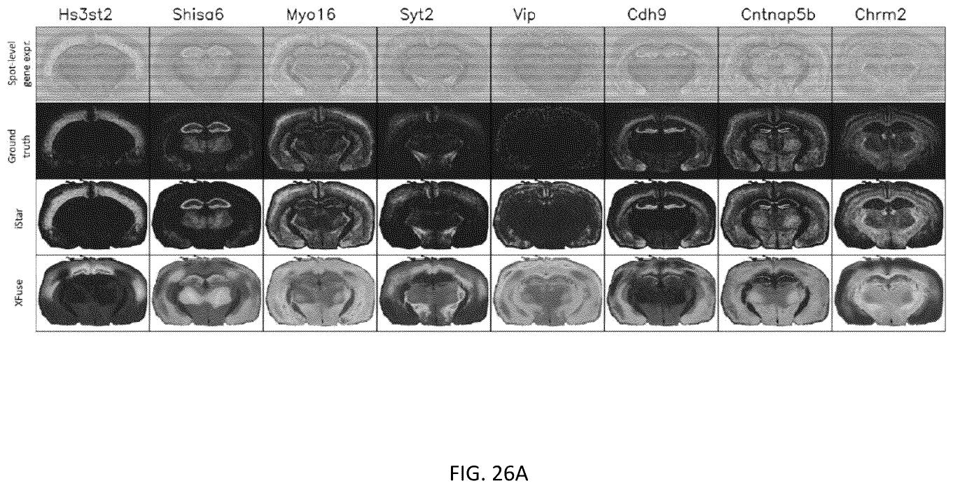

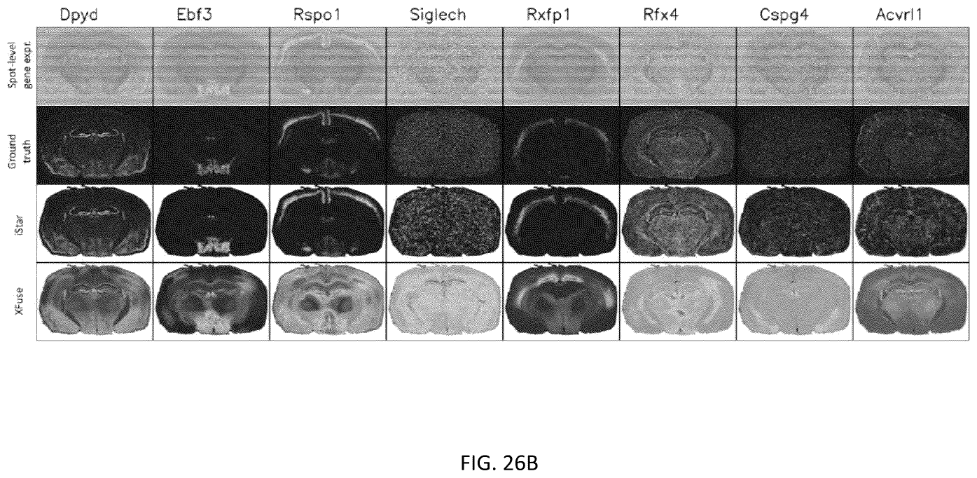

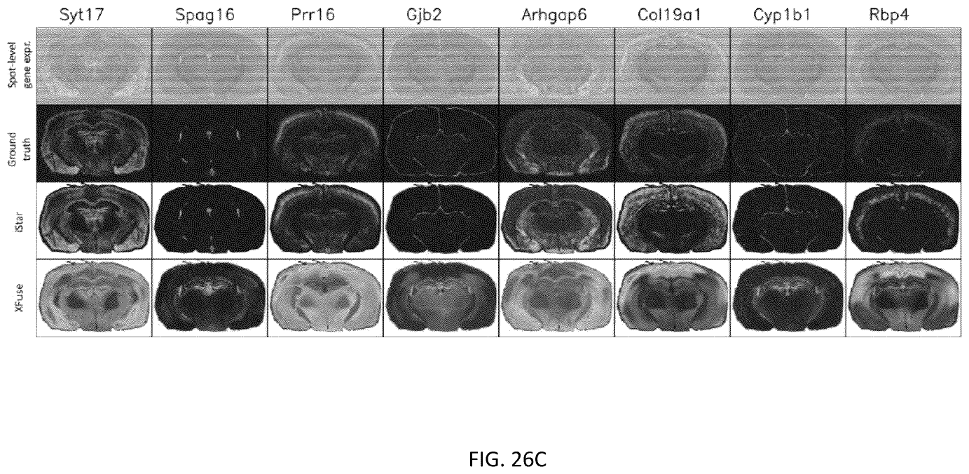

FIGS. 26A-26C show spot-level training data, ground truth gene expression, and predicted super-resolution gene expression by iStar and xFuse for 24 genes whose variance are in the 20%-40% qualities among all the 248 genes in the Xenium-derived pseudo-Visium data obtained from mouse brain. The variance quantiles of the genes in the order of top-left, top-right, bottom-left, bottom-right are equally spaced from 40% to 20% (in descending order). Super-resolution gene expressions are visualized at the scale of 8× resolution enhancement.

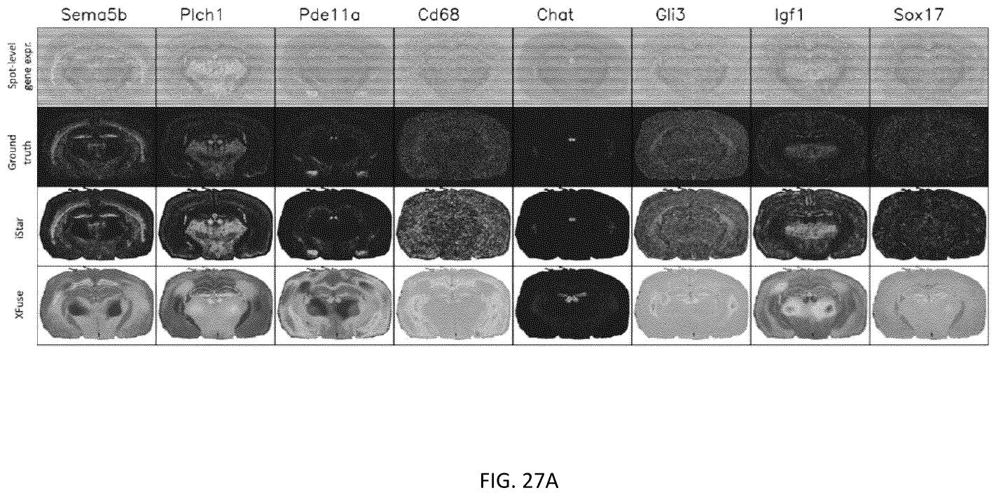

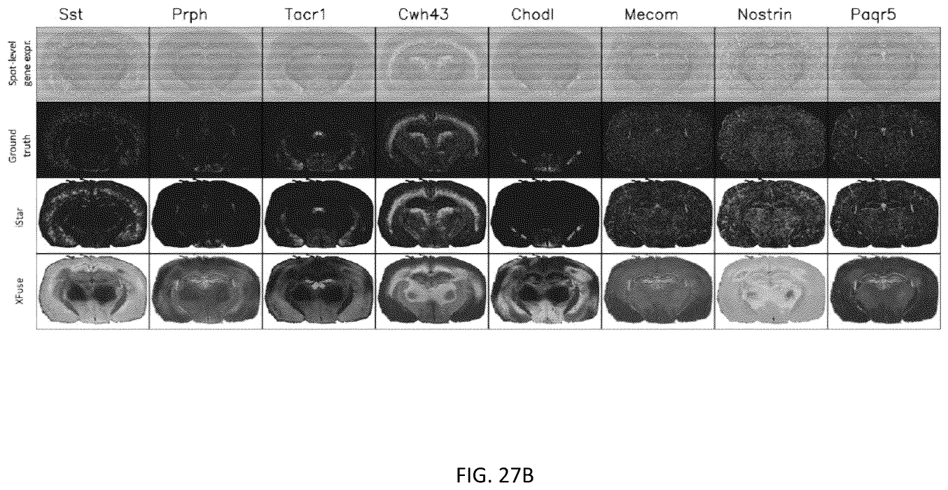

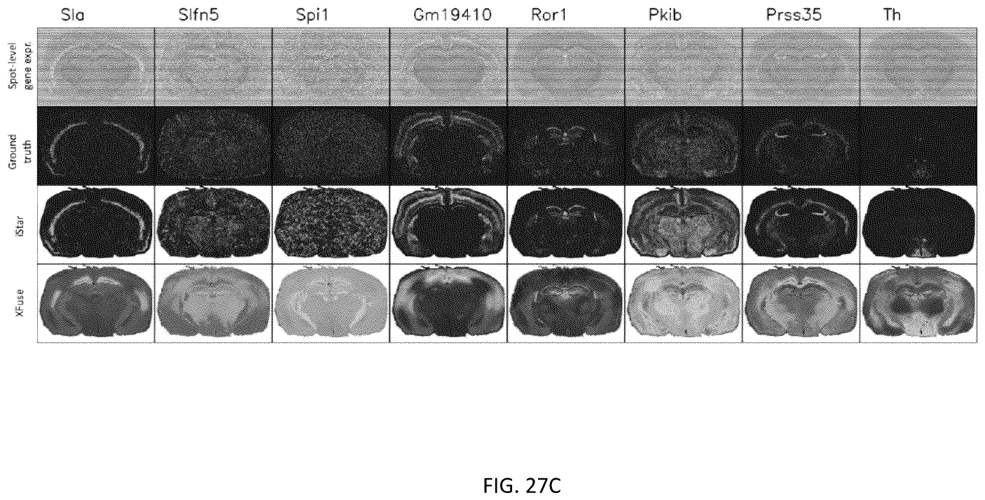

FIGS. 27A-27C show spot-level training data, ground truth gene expression, and predicted super-resolution gene expression by iStar and xFuse for 24 genes whose variance are in the 0%-20% qualities among all the 248 genes in the Xenium-derived pseudo-Visium data obtained from mouse brain. The variance quantiles of the genes in the order of top-left, top-right, bottom-left, bottom-right are equally spaced from 20% to 0% (in descending order). Super-resolution gene expressions are visualized at the scale of 8× resolution enhancement.

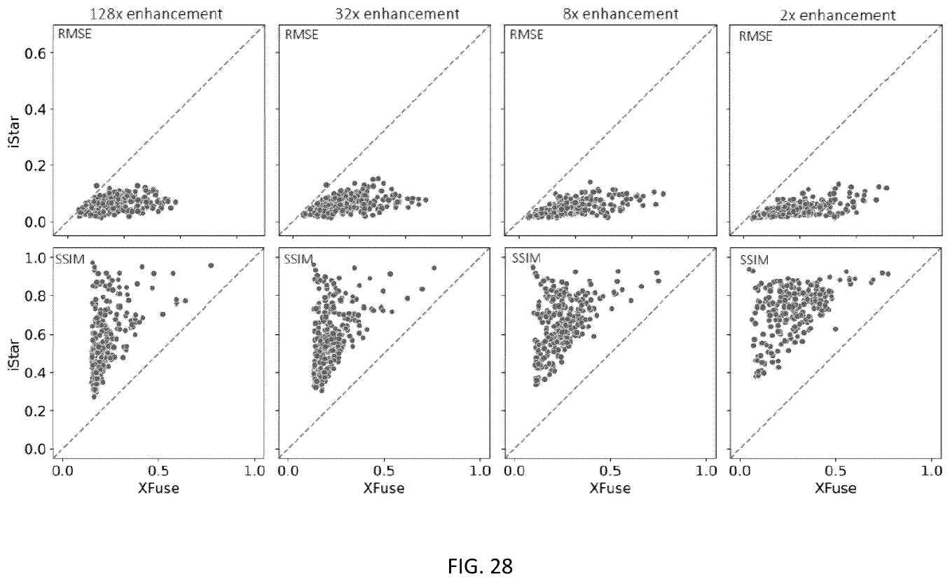

FIG. 28 shows prediction accuracy of iStar and XFuse as measured by RMSE and SSIM for all the 248 genes in the Xenium-derived pseudo-Visium data obtained from mouse brain. In each scatter plot, a dot represents one gene.

FIGS. 29A-29D show segmentation of the Xenium-derived pseudo-Visium data obtained from a mouse brain by iStar and XFuse using all 248 genes available in this dataset. FIG. 29A shows a histology image of the mouse brain. FIG. 29B shows an Allen Brain Atlas annotation of the mouse brain. FIG. 29C shows segmentation of the mouse brain using iStar super-resolution gene expression. FIG. 29D shows segmentation of the mouse brain using XFuse super-resolution gene expression. Super-resolution was performed with 128× resolution enhancement.

FIGS. 30A-30C shows analysis of a colorectal cancer Visium dataset generated by 10× Genomics. The tissue was AJCC/UICC T4aN0M0, Stage Group IIB. iStar was applied to this dataset in which the top 1,000 highly variable genes together with additional marker genes in Qi et al. (with a total of 1183 genes) were included in analysis. FIG. 30A shows a pathologist annotation of the colorectar cancer Visium dataset. FIG. 30B shows a total gene expression for each spot of the colorectar cancer Visium dataset. FIG. 30C shows an iStar segmentation of the colorectar cancer Visium dataset. Super-resolution was performed with 128× resolution enhancement.

FIGS. 31A-31C show analysis of a prostate cancer Visium dataset generated by 10× Genomics. The tissue was annotated with Adenocarcinoma, Invasive Carcinoma, Stage III, and total Gleason score is 7 by 10× Genomics. iStar was applied to this dataset in which the top 1,000 highly variable genes were included in analysis. FIG. 31A shows a pathologist annotation of the prostate cancer Visium dataset. FIG. 31B shows a total gene expression for each spot of the prostate cancer Visium dataset. FIG. 31C shows an iStar segmentation of the prostate cancer Visium dataset. Super-resolution was performed with 128× resolution enhancement.

FIGS. 32A-32F show an analysis of a prostate cancer Visium dataset generated by Erickson et al. iStar was applied to this dataset in which the top 1,000 highly variable genes were included. FIG. 32A shows a histology image of the prostate cancer Visium dataset. FIG. 32B shows a total gene expression for each spot of the prostate cancer Visium dataset. FIG. 32C shows a pathologist annotation of the prostate cancer Visium dataset. FIG. 32D shows a clone annotation of the prostate cancer Visium dataset. FIG. 32E shows a clonal tree of the prostate cancer Visium dataset. FIG. 32F shows an iStar segmentation of the prostate cancer Visium dataset. The red cluster in iStar segmentation matched clones I and K, which are closely related based on the clonal tree reported in the original publication. Super-resolution was performed with 128× resolution enhancement in sample H1_4.

FIGS. 33A-33C show an analysis of a kidney cancer Visium dataset generated by Meylan et al. iStar was applied to this dataset in which the top 1,000 highly variable genes were included in analysis. TLSs were detected based on TLS score calculated using TLS marker genes in Table 2. FIG. 33A shows an iStar segmentation of the kidney cancer Visium dataset. FIG. 33B shows an iStar TLS score of the kidney cancer Visium dataset. FIG. 33C shows a manual TLS annotation of the kidney cancer Visium dataset. Super-resolution was performed with 128× resolution enhancement.

FIGS. 34A-34D show an analysis of a mouse kidney Visium dataset generated by 10× Genomics. iStar was applied to this dataset in which the top 1,000 highly variable genes were included in analysis. FIG. 34A shows a histology image of the mouse kidney Visium dataset. FIG. 34B shows a total gene expression for each spot of the mouse kidney Visium dataset. FIG. 34C shows an iStar segmentation of the mouse kidney Visium dataset. FIG. 34D shows an anatomy of the mouse kidney Visium dataset. Super-resolution was performed with 128× resolution enhancement.

FIGS. 35A-35D show an analysis of a coronal section of a mouse brain Visium dataset generated by 10× Genomics. iStar was applied to this dataset in which the top 1,000 highly variable genes were included in analysis. FIG. 35A shows a histology image of the coronal section of the mouse brain Visium dataset. FIG. 35B shows a total gene expression for each spot of the coronal section of the mouse brain Visium dataset. FIG. 35C shows an iStar segmentation of the coronal section of the mouse brain Visium dataset. FIG. 35D shows a hippocampus of the coronal section of the mouse brain Visium dataset. FIG. 35E shows an Allen Brain Atlas annotation of the coronal section of the mouse brain Visium dataset. Super-resolution was performed with 128× resolution enhancement. Zoomed-in examination of iStar's segmentation result in hippocampus indicates iStar was able to identify CA1, CA2, and CA3, which are hard to distinguish from the histology image.

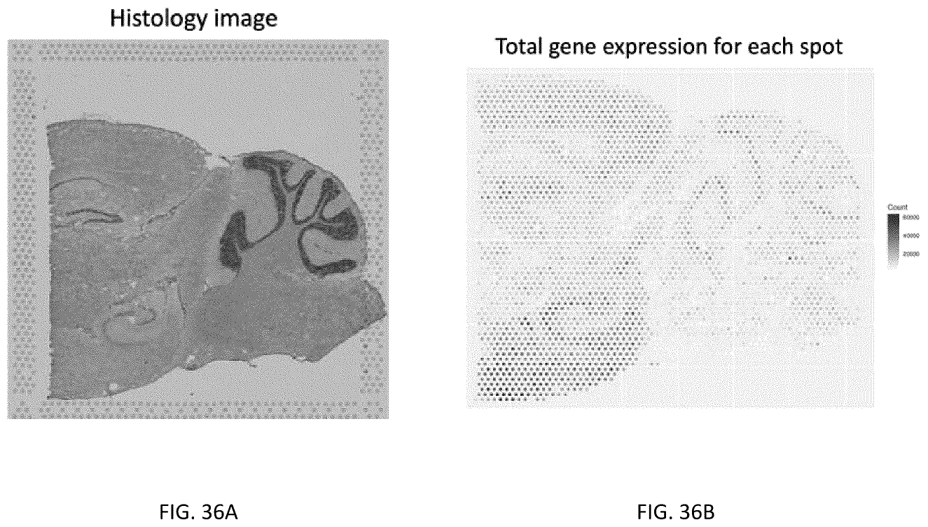

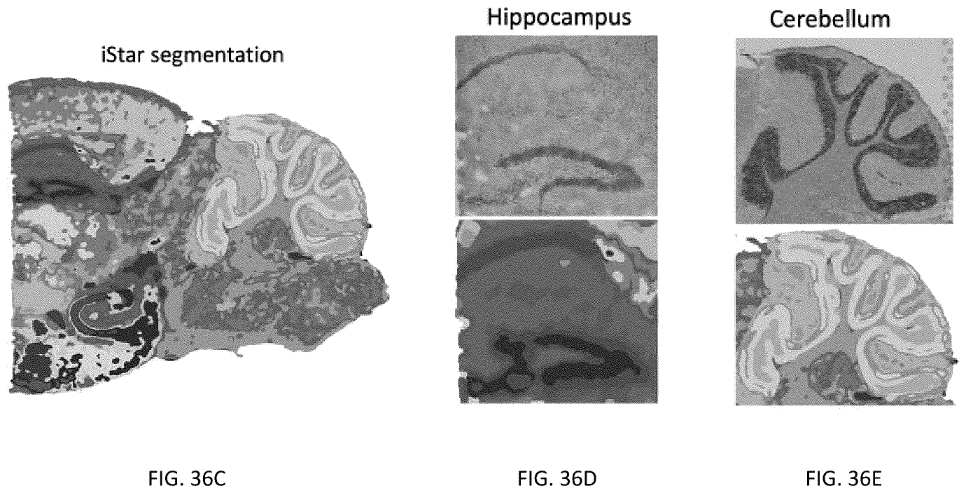

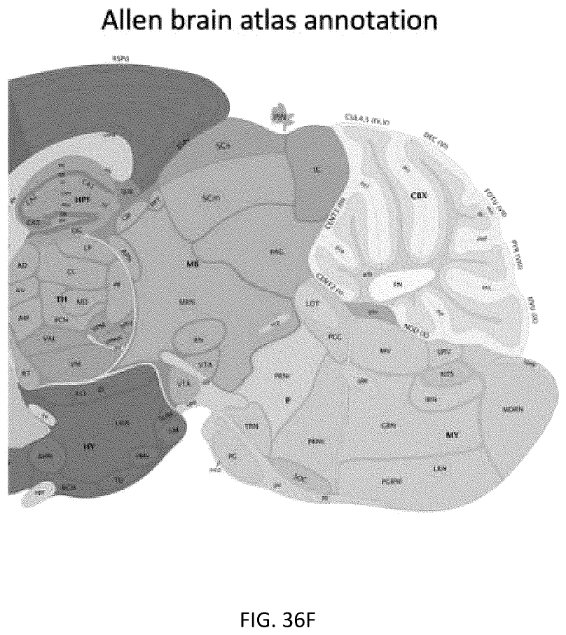

FIGS. 36A-36F show an analysis of a posterior section of a mouse brain Visium dataset generated by 10× Genomics. iStar was applied to this dataset in which the top 1,000 highly variable genes were included in analysis. FIG. 36A shows a histology image of the posterior section of the mouse brain Visium dataset. FIG. 36B shows a total gene expression for each spot of the posterior section of the mouse brain Visium dataset. FIG. 36C shows an iStar segmentation of the posterior section of the mouse brain Visium dataset. FIG. 36D shows a hippocampus of the posterior section of the mouse brain Visium dataset. FIG. 36E shows a cerebellum of the posterior section of the mouse brain Visium dataset. FIG. 36F shows an Allen Brain Atlas annotation of the posterior section of the mouse brain Visium dataset. Zoomed-in examination of iStar's segmentation result in cerebellum indicates iStar was able to reveal fine-grained tissue structure in this region that agreed with Allen Brain Atlas annotation.

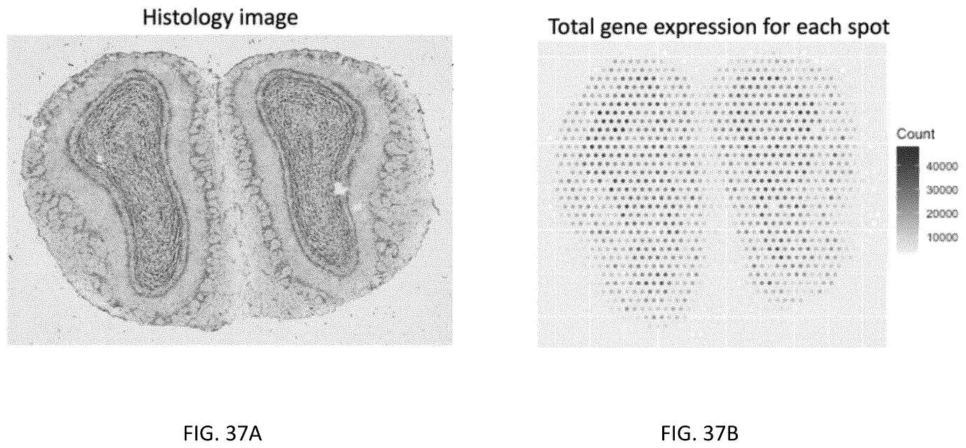

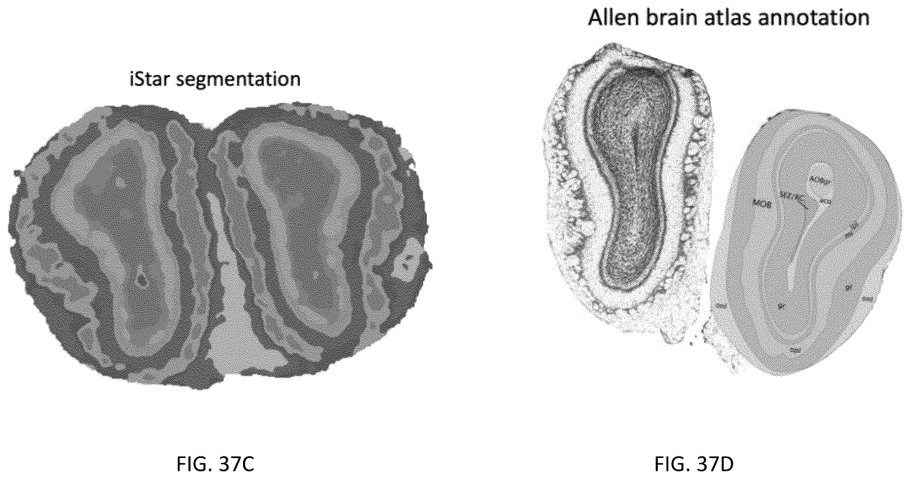

FIGS. 37A-37D show an analysis of a mouse brain (olfactory bulb) Visium dataset generated by 10× Genomics. iStar was applied to this dataset in which the top 1,000 highly variable genes in analysis. Super-resolution was performed with 128× resolution enhancement. iStar was able to reveal fine-grained tissue structure in olfactory bulb that agreed with Allen Brain Atlas annotation. FIG. 37A shows a histology image of the olfactory bulb of the mouse brain Visium dataset. FIG. 37B shows a total gene expression for each spot of the olfactory bulb of the mouse brain Visium dataset. FIG. 37C shows an iStar segmentation of the olfactory bulb of the mouse brain Visium dataset. FIG. 37D shows an Allen Brain Atlas annotation of the olfactory bulb of the mouse brain Visium dataset.

FIG. 38 shows a graph comparing levels of transcriptome-wide gene coverage and special resolution of iStar and other sources. iStar is a computational approach that reconstructs super-resolution spatial transcriptomics (ST) data with transcriptome-wide gene coverage by integrating ST data generated from sequencing-based ST platforms, such as Visium, with high-resolution histology images. Although ST platforms like Xenium, CosMx, and MERSCOPE have single-cell resolution, they rely on pre-designed gene panels and lack whole-transcriptome coverage. iStar, on the other hand, does not impose any extra experimental cost but can substantially increase the gene expression resolution for sequencing-based ST data to near-single-cell resolution.

FIG. 39 compares a goodness of fit of iStar and XFuse as measured by the in-sample spot-level RMSE and Pearson's correlation on the training. This evaluation was based on the Xenium-derived pseudo-Visium data and Subject G in the HER2ST breast cancer data.

FIGS. 40A-40B show a goodness of fit of iStar for all datasets analyzed herein as measured by the in-sample spot-level RMSE and Pearson's correlation on the training, respectively.

FIG. 41 shows the impact of the confidence threshold for marker gene-based cell type annotation in iStar. Shown is the result for the analysis of sample H1 in the HER2ST breast cancer dataset.

FIGS. 42A-42B show a super-resolution gene expression prediction accuracy of iStar and XFuse as measured by Pearson's correlation coefficient and Pearson's correlation coefficient stratified by gene expression variance, respectively. Super-resolution gene expression prediction accuracy of iStar and XFuse as measured by Pearson Correlation Coefficient (PCC) for all the 313 genes in the Xenium derived pseudo-Visium data obtained from a breast cancer patient (top), stratified by gene expression variance (bottom). In each scatter plot, a dot represents one gene. In each box plot (center line: median, box limits: upper and lower quartiles, whiskers: 1.5 interquartile range), the x-axis represents the quantile of the expression variance, where Stratum 1 to Stratum 5 correspond to quantiles 0.0-0.2, 0.2-0.4, . . . , 0.8-1.0. In this analysis, Section 1 was first treated as the training sample in which in-sample prediction was performed for Section 1 and then out-of-sample prediction was performed for Section 2. The analysis was then repeated by treating Section 2 as the “in-sample” and Section 1 as the “out-of-sample”. The evaluation metrics in “Average” is the average of those in Sections 1 and Section 2.

FIG. 43 shows super-resolution gene expression prediction accuracy of iStar and XFuse as measured by Pearson correlation coefficient for all the 248 genes in the Xenium-derived pseudo-Visium data obtained from mouse brain (top), stratified by gene expression variance (bottom). In each scatter plot, a dot represents one gene. In each box plot (center line: median, box limits: upper and lower quartiles, whiskers: 1.5 interquartile range), the x-axis represents the quantile of the expression variance, where Stratum 1 to Stratum 5 correspond to quantiles 0.0-0.2, 0.2-0.4, . . . , 0.8-1.0.

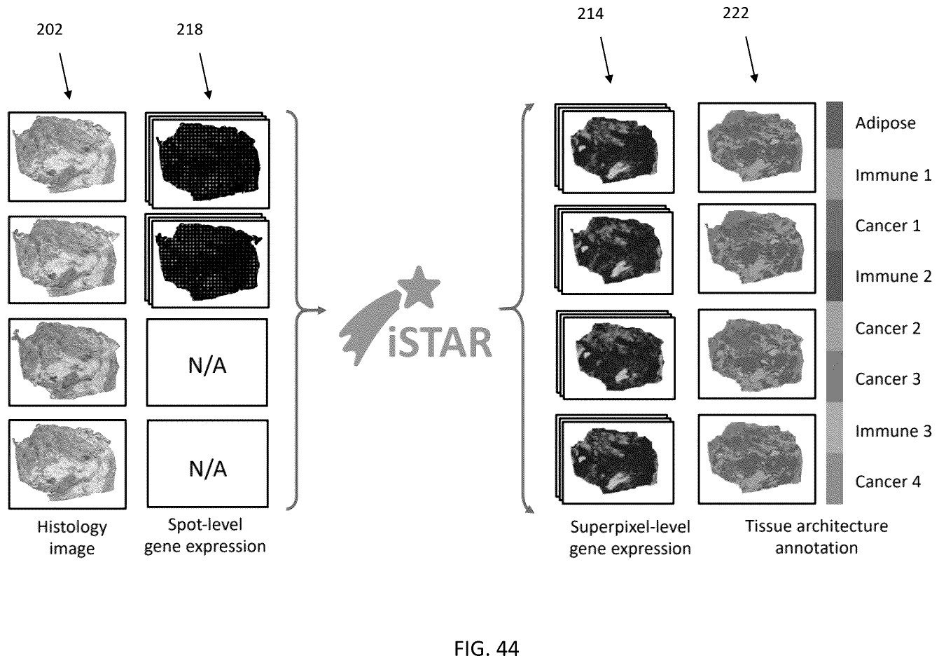

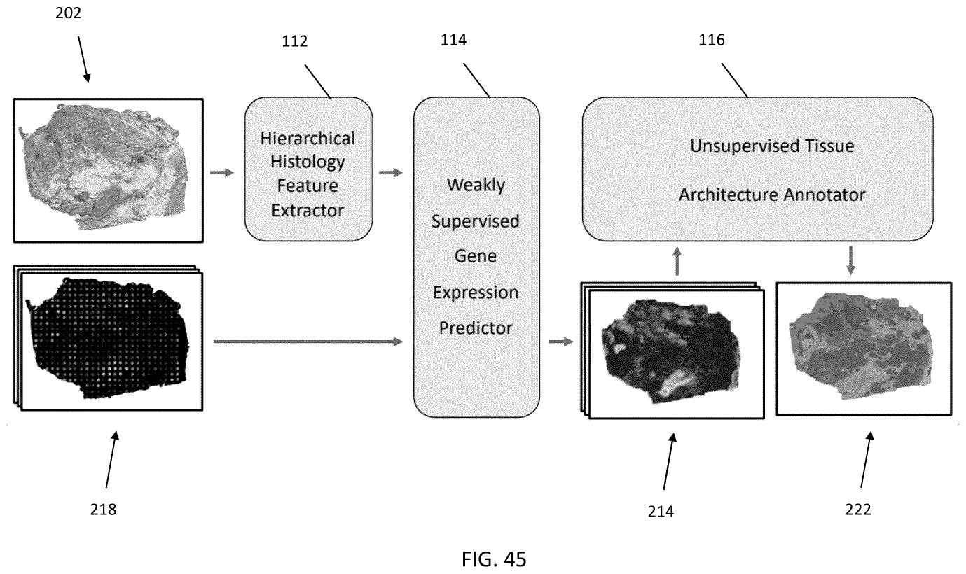

FIGS. 44 and 45 show workflows of iStar, wherein iStar uses histology images 202 and spot-level gene expressions 218 to predict super-resolution gene expressions 214 and automatically generate tissue architecture annotations 222. Histology feature extractor 112 receives histology images 202, partitions them, and extracts histology features from them. Super-resolution gene expression predictor 114, which can include a weakly supervised model, receives the extracted features and spot-level gene expressions 218 to predict super-resolution gene expressions 214, which tissue architecture annotator 116, which can implement an unsupervised model, receives to automatically generate tissue architecture annotations 222.

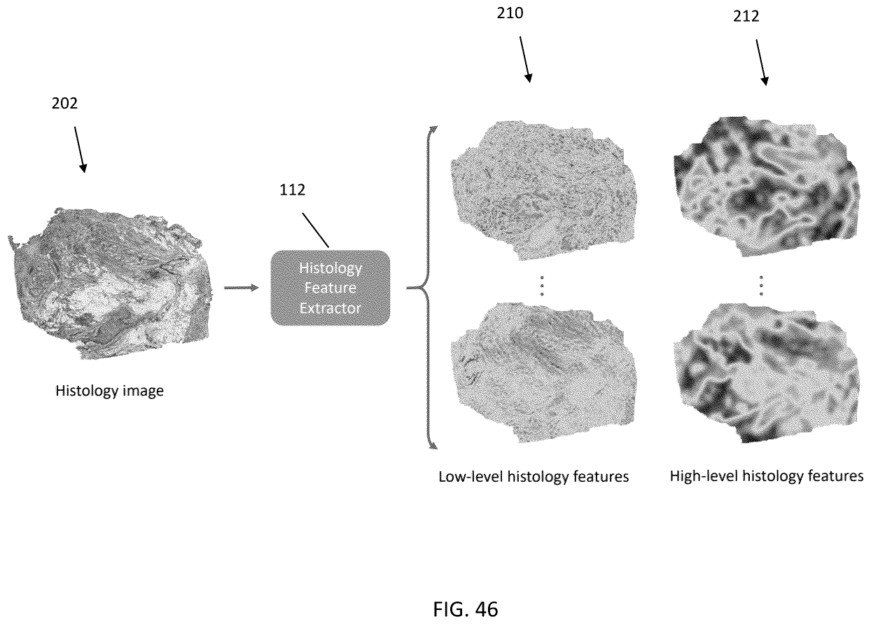

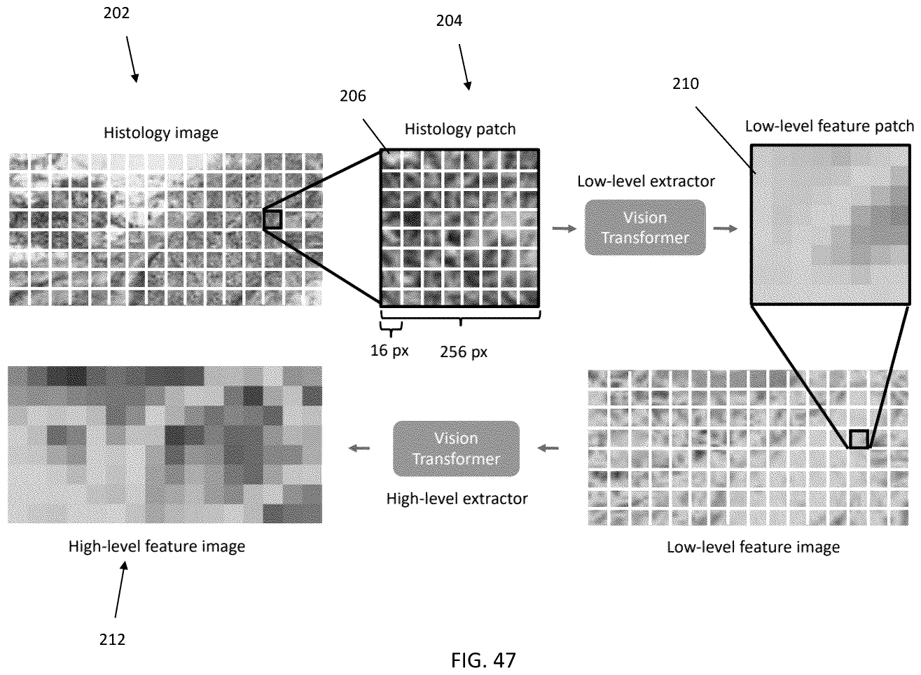

FIGS. 46 and 47 show workflows of feature extractor 112, which receives histology images 202 and extracts low-level image features 210 (or low-level histology features) from image sub-tiles 206 partitioned from the histology images 202 and high-level image features 212 (or high-image histology features) from image tiles 204 partitioned from the histology images 202. Feature extractor 112 can implement a low-level extractor including a vision transformer to extract low-level image features 210 and a high-level extractor including a vision transformer to extract high-level image features 212.

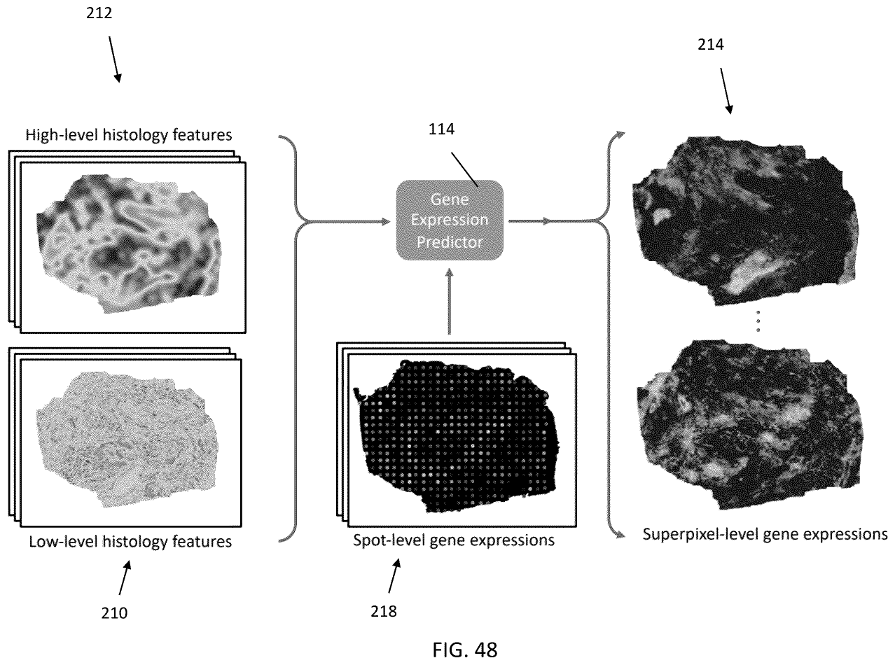

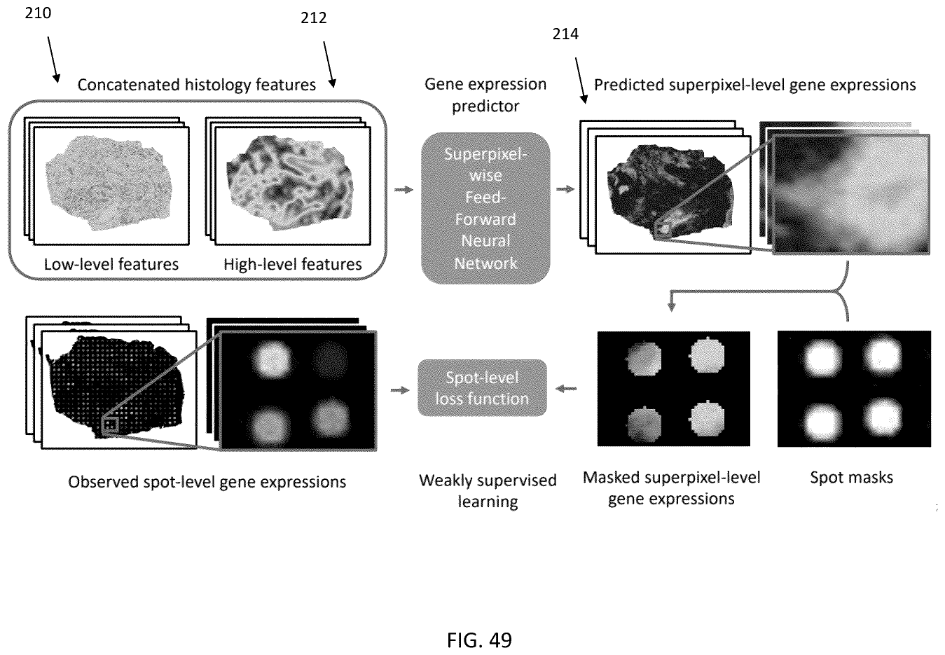

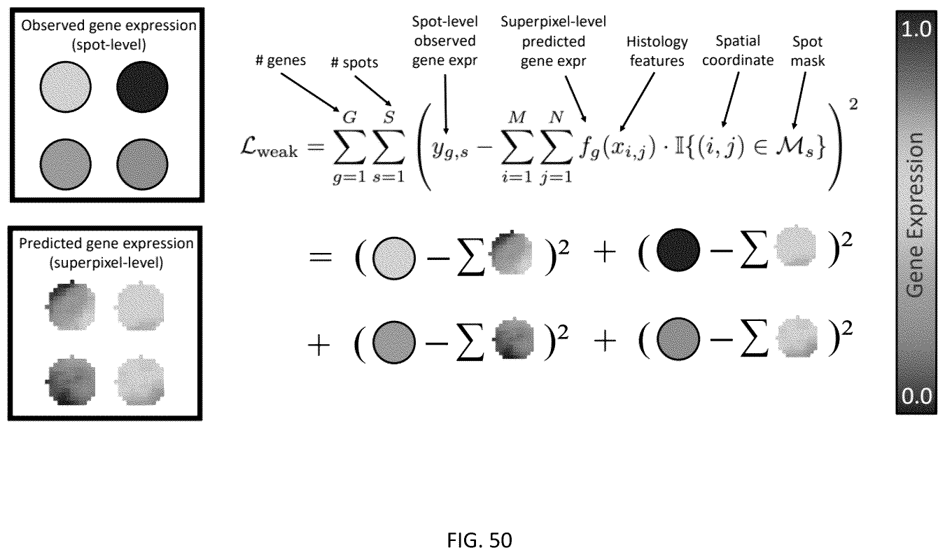

FIGS. 48 and 49 show workflows of super-resolution gene expression predictor 114. Super-resolution gene expression predictor 114 receives low-level image features 210 and high-level image features 212, which were extracted from the partitioned histology image, and spot-level gene expressions 218 to predict super-resolution gene expressions 214. FIG. 50 shows a workflow of a spot-level weakly supervised learning that trains a feed-forward neural network for super-resolution gene expression predictor 114 shown in FIGS. 1 and 2.

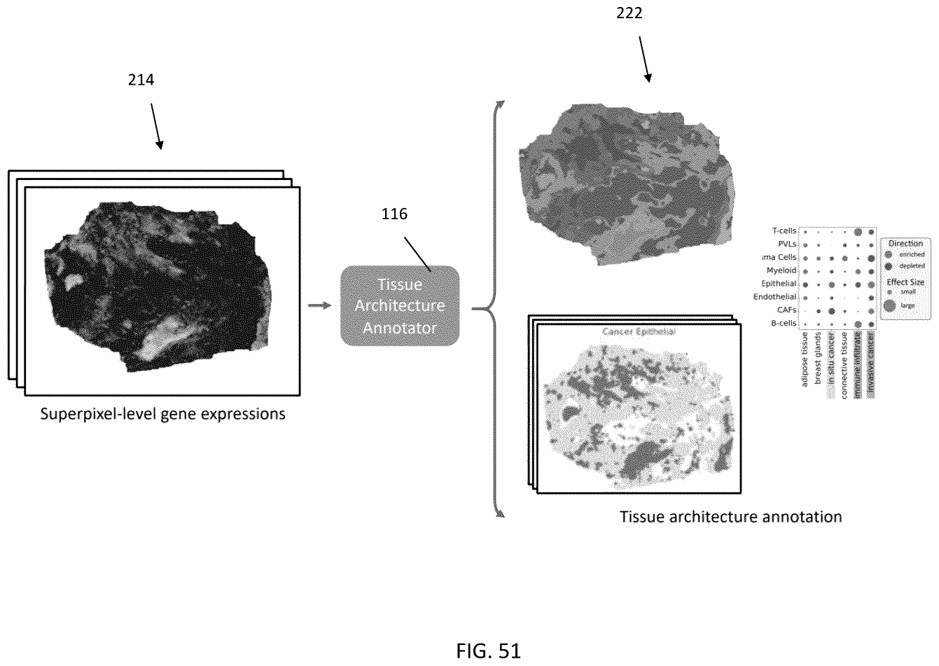

FIGS. 51 and 52 show a workflow of tissue architecture annotator 116, which receives super-resolution gene expressions 214 to automatically generate tissue architecture annotations 222. The Gaussian filter smoothed gene expression embeddings obtained from the second last layer of the feed-forward neural network in the gene expression predictor. The smoothed embeddings were then used as inputs for K-means clustering. Also from super-resolution gene expressions 214, cell types are predicted at a super-pixel level and a cell type enrichment analysis is conducted. The super-pixels are clustered based on their cell type enrichment.

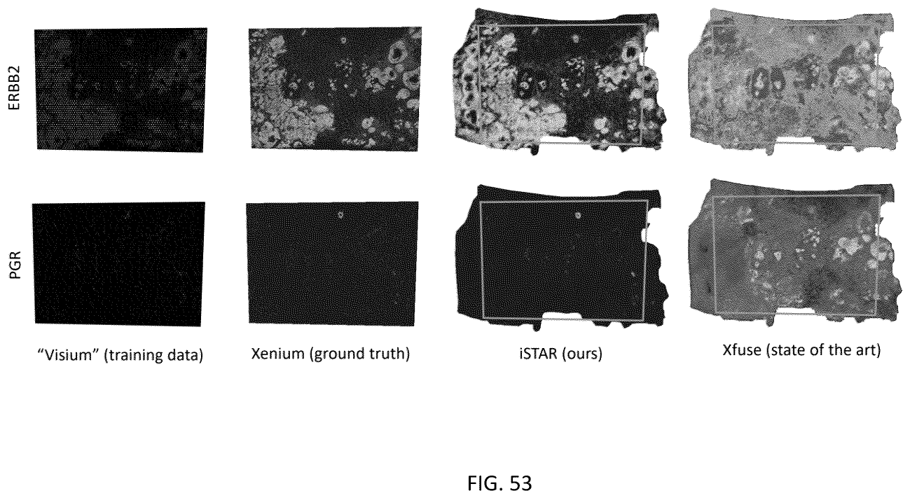

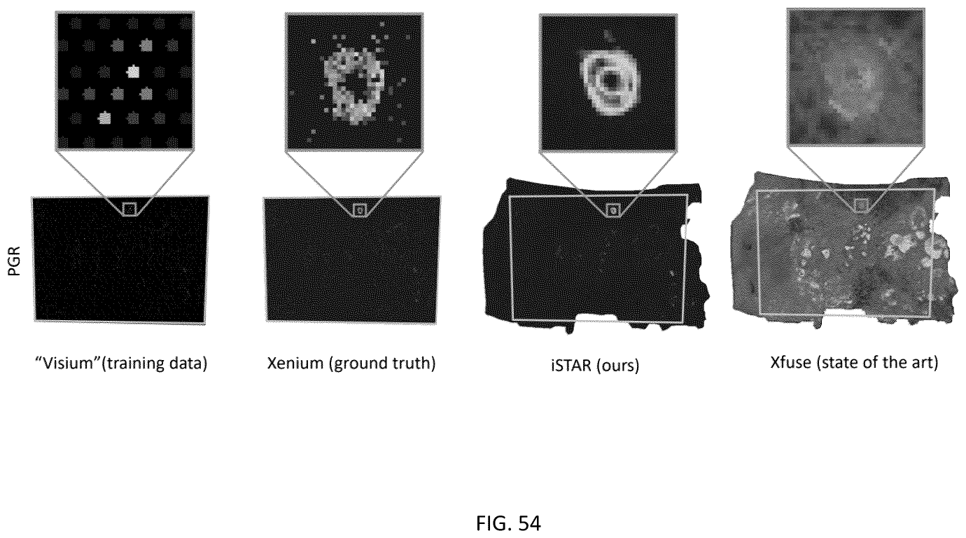

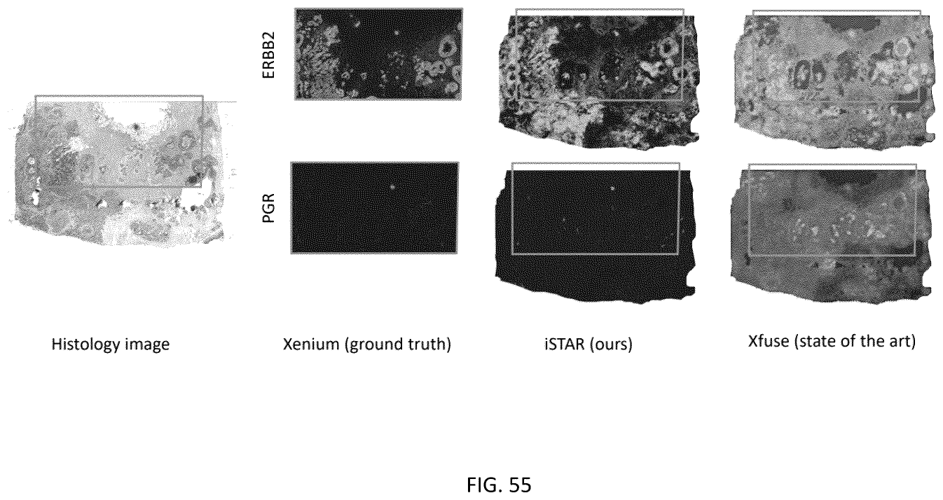

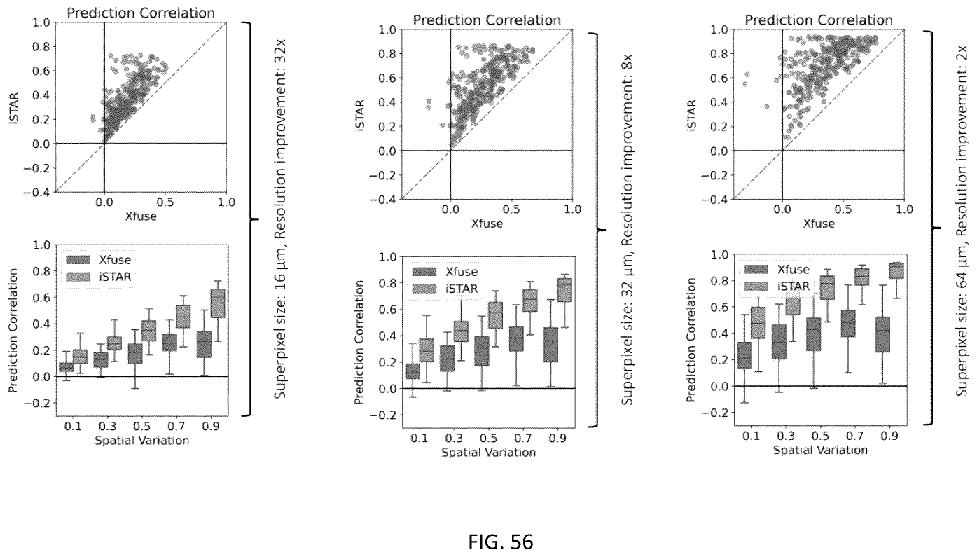

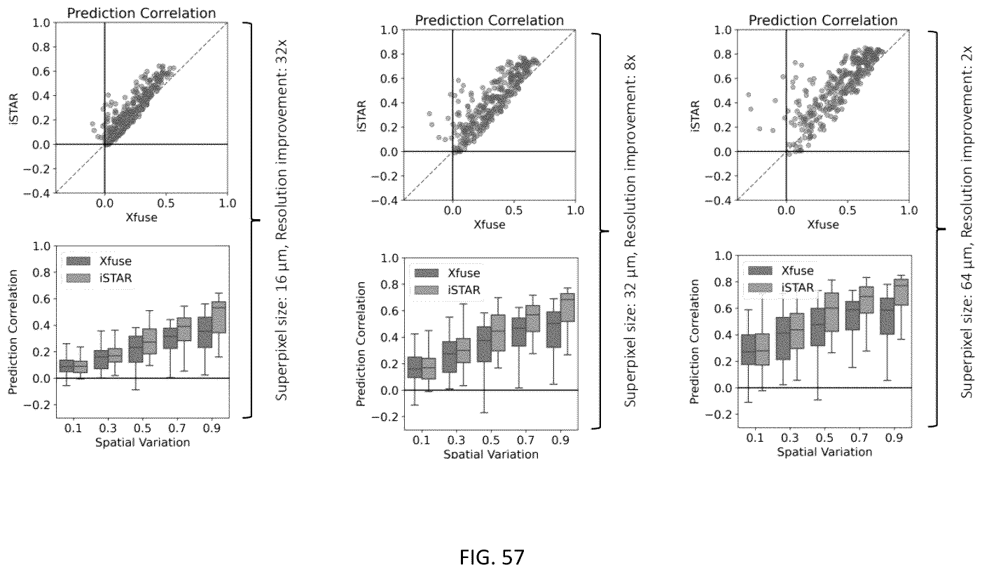

FIG. 53 shows in-sample gene expression predictions of ERBB2 and PRG by Visium, Xenium, iStar, and XFuse. FIG. 54 shows in-sample gene expression predictions of PRG by Visium, Xenium, iStar, and XFuse. FIG. 55 shows out-of-sample gene expression predictions of ERBB2 and PRG by Xenium, iStar, and XFuse. FIGS. 56 and 57 show in-sample and out-of-sample prediction accuracy, respectively, of iStar and XFuse;

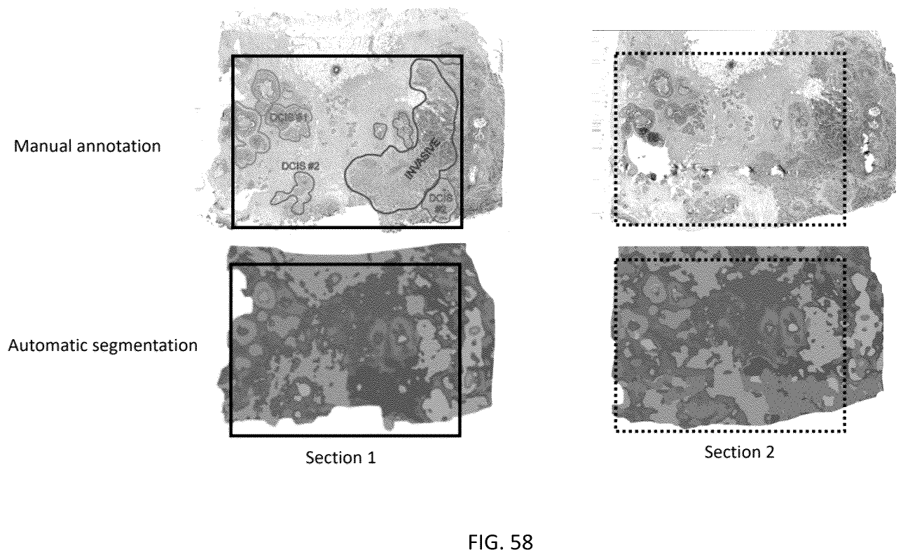

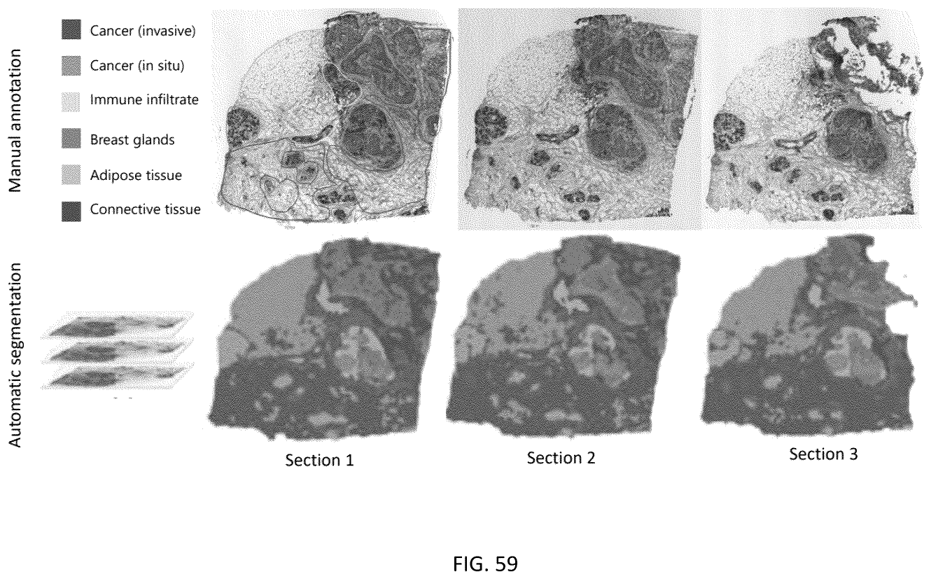

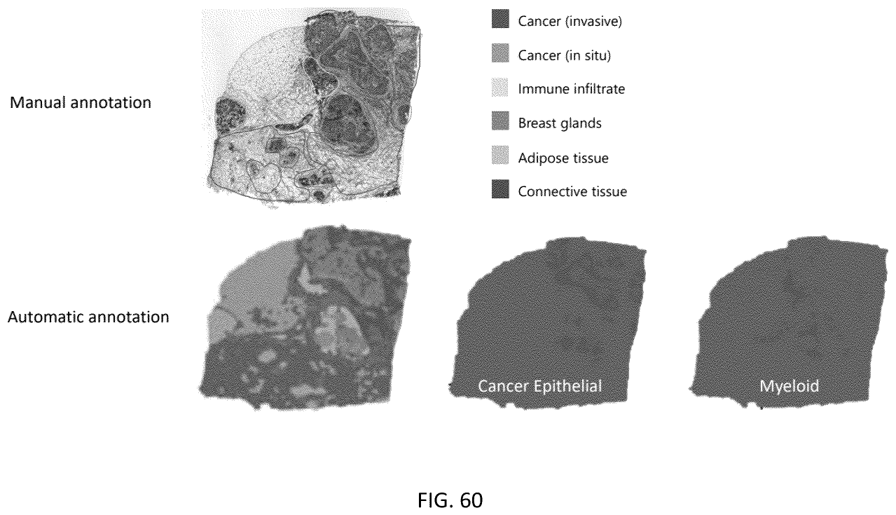

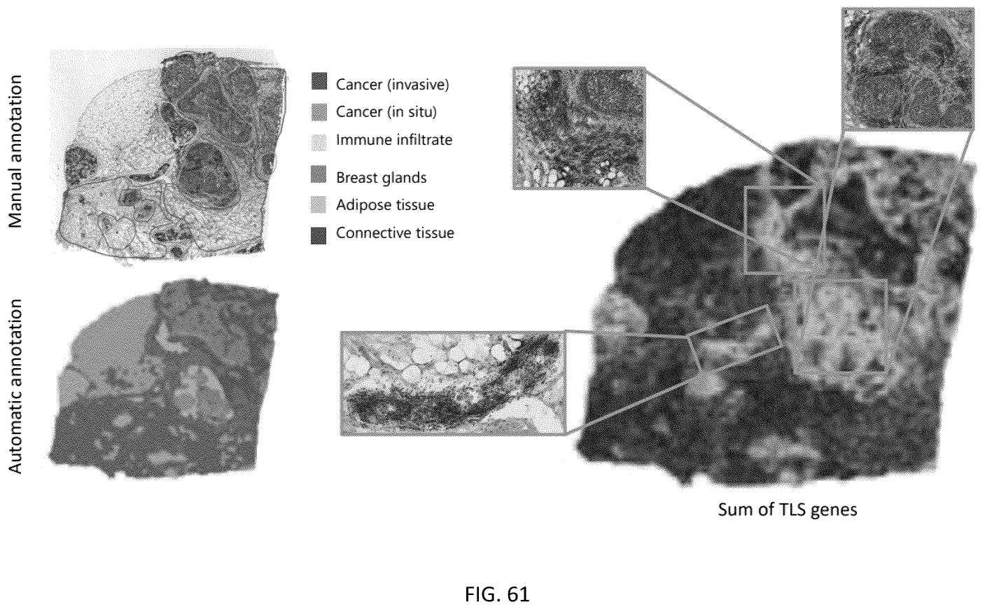

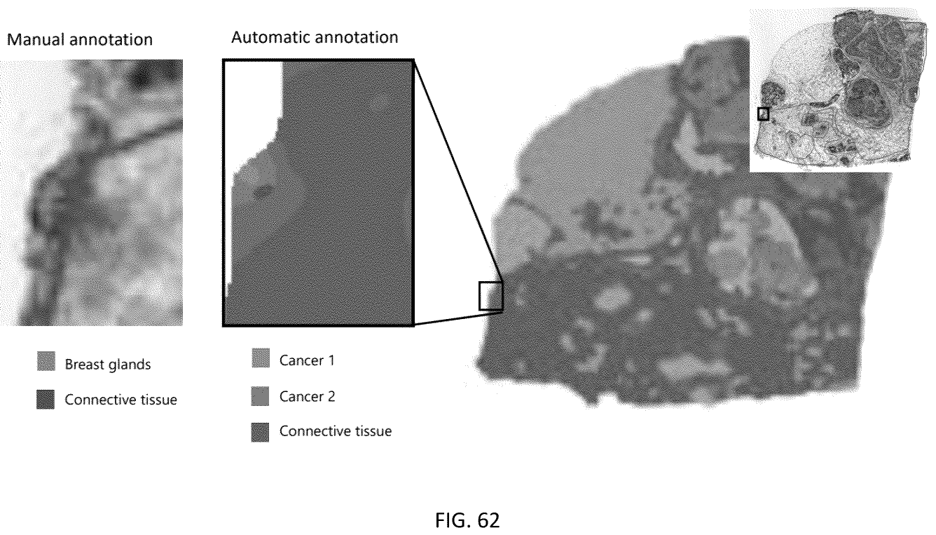

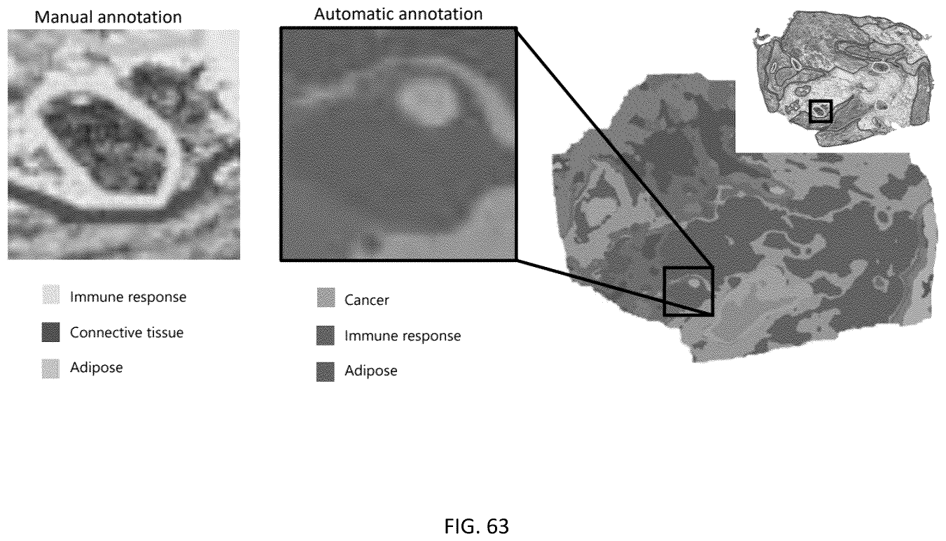

FIGS. 58 and 59 show registration-free 3D tissue segmentation by iStar. Tissue architecture annotator 116, as shown in FIGS. 1 and 2, can bypass the image registration step. FIG. 60 shows an example of automatic tissue annotation by iStar. FIG. 61 shows TLS detection from automatic tissue annotation by iStar. FIG. 62 shows an example of precision from automatic annotation by iStar. FIG. 63 shows an example of precision from automatic annotation by iStar. The automatic annotation by iStar can detect cancer regions with computed annotation that is overlooked by manual annotation.

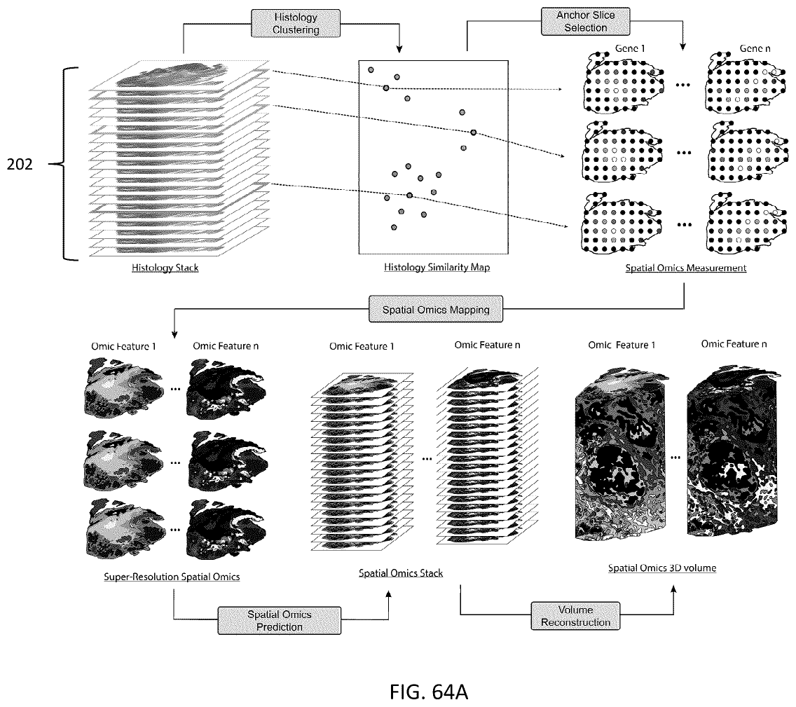

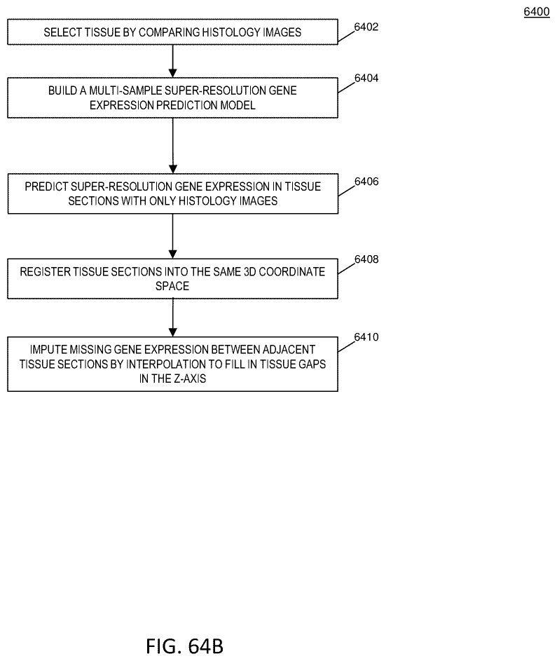

FIG. 64A shows a flow diagram of a process for inferring super-resolution tissue architecture for a three-dimensional (3D) tissue volume. The at least one histology image 202, as shown in FIGS. 1 and 2, of the tissue sample can include a plurality of histology images 202 that iStar utilizes to infer super-resolution tissue architecture for a 3D tissue volume rather than just a 2D tissue image based on a single histology image 202. Thin slices of a tissue sample are cut and histology images 202 of the slices are generated. Each of histology images 202 is of a distinct tissue slice of the tissue sample. iStar can align histology images 202 and identify representative histology images 202 of the distinct tissue slices, also referred to herein as anchor slices, and discard the histology images 202 that are redundant or near redundant to the representative histology images 202. As an example, iStar can reduce twenty histology images 202 to about three histology images 202, which significantly reduces computation time. iStar imputes gene expressions between the representative histology images 202 to infer super-resolution tissue architecture for the 3D tissue volume.

First, histology images 202 are aligned by first extracting deep histology features from the histology images 202 using a feature extractor, such as histology feature extractor 112 shown in FIGS. 1 and 2, which is trained by self-supervised learning. Histology feature extractor 112 converts the RGB value of each pixel in histology images 202 to a vector of n histology features, which turns the 3-channel RGB histology image 202 into an n-channel histology feature image. Then key pairs are identified between every two adjacent 2D histology slices (i.e., histology images 202 of adjacent tissue slices). For every key pair between histology slices S1 and S2, the key pair consists of a location in S1 and a location in S2. Then optimization is performed to find the best deformation of each slice that minimizes the distance between each key pair.

FIG. 64B is a flow diagram of an example method 6400 for inferring super-resolution tissue architecture for a 3D tissue volume. At step 6402, tissue is selected by comparing histology images, such as hematoxylin-and-eosin-stained histology images or any other type of histology images known in the art. At step 6404, a multi-sample super-resolution gene expression prediction model is built. At step 6406, super-resolution gene expression in tissue sections is predicted with only histology images using the built model. At step 6408, tissue sections are registered into the same 3D coordinate space. At step 6410, missing gene expression between adjacent tissue sections is imputed by interpolation to fill in tissue gaps in the z-axis.

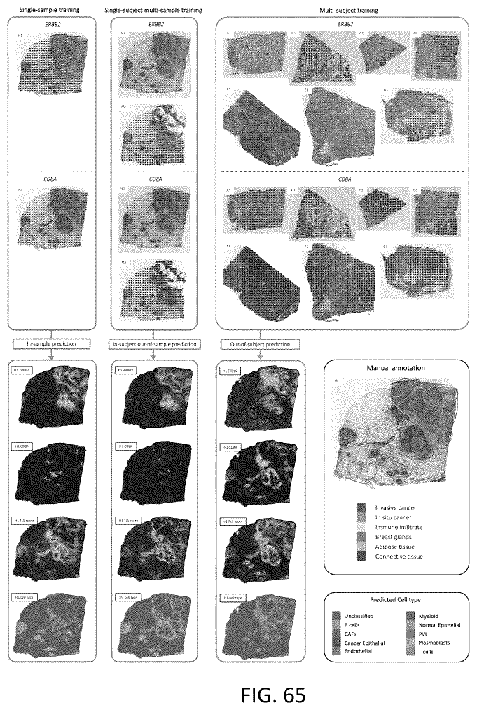

Tissues across subjects are prone to batch effects. When training a model that predicts spatial gene expression (SGE) from histology, batch effects can substantially mislead the model and hamper the prediction performance. The key to solve the batch effect problem is to expose the model to a diverse range of samples during training. To maximize the histology-SGE training sample size, we adopt the following approach. Datasets are collected that contain samples with both histology and non-SGE (nSGE) information. A first model is trained that predicts the nSGE features from histology images. Next, datasets that contain samples with both nSGE and SGE information are collected. A second model is trained that predicts SGE features from nSGE features. Then, the first and second models are combined, which yields a third model that predicts SGE from histology. Finally, the third model is used to initialize a fourth and final model for predicting SGE from histology. After initialization, this final model is trained on publicly available datasets that contain both histology and SGE information. iStar can predict gene expression in biobank tissue samples to allow the integration with electronic medical record information and to build diagnostic and predictive models for disease. Thus, iStar can train with a subject's histology and gene expression data and predict a new subject's gene expression using the new subject's histology data. The predictor model can be trained with spot-level gene expression observations of at least one training subject, wherein the at least one training subject is distinct from a source of the tissue sample. FIG. 65 shows out-of-subject gene expression prediction, where the model was trained using spot-level gene expression of subjects independent from the testing tissue section, in-sample prediction, where the model was trained using spot-level gene expression of the testing tissue section, and the in-subject out-of-sample prediction, where the model was trained using spot-level gene expression of the tissue sections that are from the same subject as and are adjacent to the testing tissue section. Shown are the predicted gene expression of ERBB2 and CD8A, as well as the predicted cell type and tertiary lymphoid structure (TLS) score.

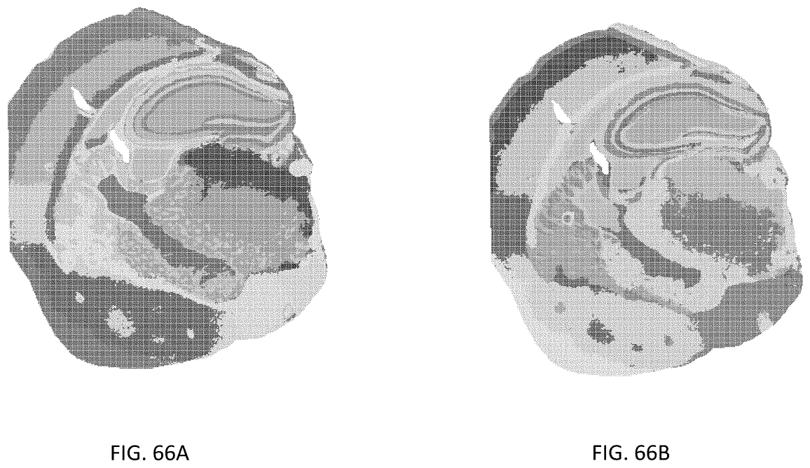

iStar can include an integration of multi-modal spatial omics. iStar can predict near-single-cell resolution intensity for metabolomics data. For example, super-resolution gene expression and resulting annotation can be at a near single-cell level rather than a square-shaped superpixel level as described herein. When combined with a prediction model for transcriptomics data, this yields multi-modal spatial omics data that include both metabolite and gene expression information. This multi-modal prediction approach can also be applied to other omics modalities, such as proteomics, lipid omics, and epigenetics. FIG. 66A shows tissue segmentation using metabolomics data with enhanced spatial resolution. FIG. 66B shows tissue segmentation using both metabolomics and transcriptomics data with enhanced spatial resolution. iStar first identifies the associations between different data modalities in existing databases. For example, if the goal is to integrate gene expression and protein expression data, existing databases can be combined from transcriptome-wide association studies and proteome-wide association studies to identify the associations between each gene's gene expression level and protein expression level. When training the prediction model, the identified association relationships are incorporated into the objective function, so that after training, the association relationships of the predicted multi-modal spatial omics resemble the association relationships obtained from existing databases.

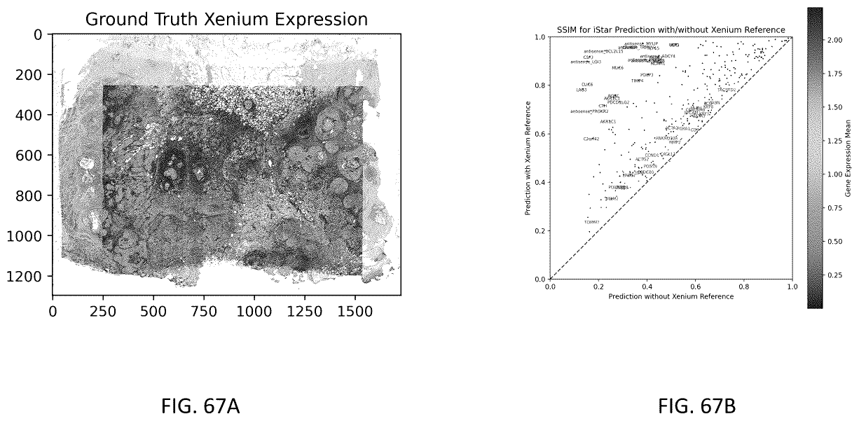

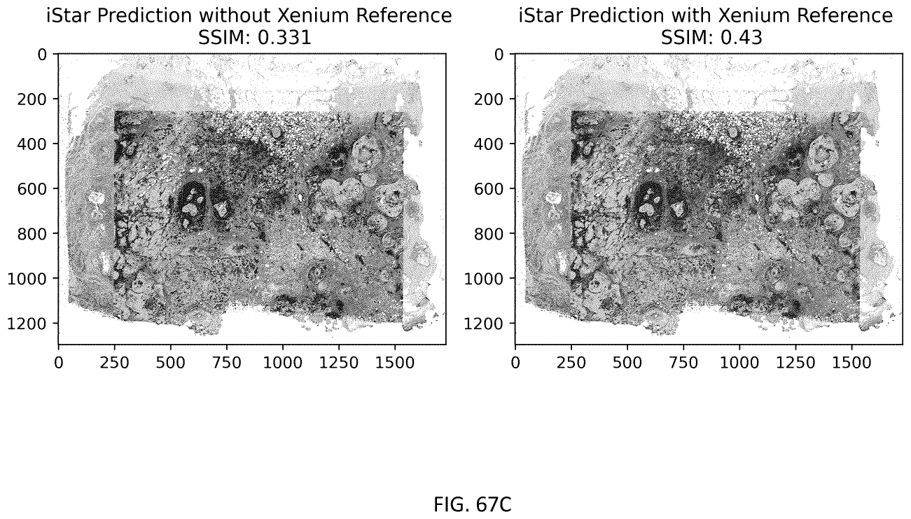

Many newer spatial transcriptomics platforms are now available on the market, creating a need to integrate different spatial transcriptomics data types for gene expression prediction. iStar can incorporate both Visium and Xenium data to build a gene expression prediction model. This model yields more accurate gene expression predictions than a model trained with data from a single platform, such as only Visium data. iStar can also incorporate data generated from other platforms, such as Visium HD. Some platforms provide high gene coverage and low resolution or low gene coverage and high resolution. Some platforms provide expressions for only a few genes while other platforms provide expressions for thousands of genes. Platforms also vary in the region size and shape for providing gene expression. Since each platform has unique strengths and weaknesses, using data from multiple platforms to train iStar balances the platform-specific weaknesses and makes iStar more adaptable to input from various platforms. The model can be trained for predicting gene expression coverage in two stages. The model can be trained using low gene coverage data and then with high gene coverage data. FIG. 67A shows a ground truth Xenium expression. FIG. 67B shows the SSIM of iStar predictions with and without Xenium expression. Visium+Xenium combined training data yield higher gene expression prediction accuracy. FIG. 67C shows iStar prediction having a SSIM of 0.331 without the Xenium reference and iStar prediction having a SSIM of 0.43 with the Xenium reference.

### Computational Efficiency

Computational efficiency was another area that iStar significantly outperformed XFuse. In the benchmark experiments, iStar was approximately 200 times faster than XFuse. The end-to-end analysis of a typical dataset by iStar usually finishes within ten minutes, while XFuse took about a day. The detailed runtimes for training and prediction are reported in Table 1. Experiments were conducted on an NVIDIA Geforce RTX 2080 Ti graphics card.

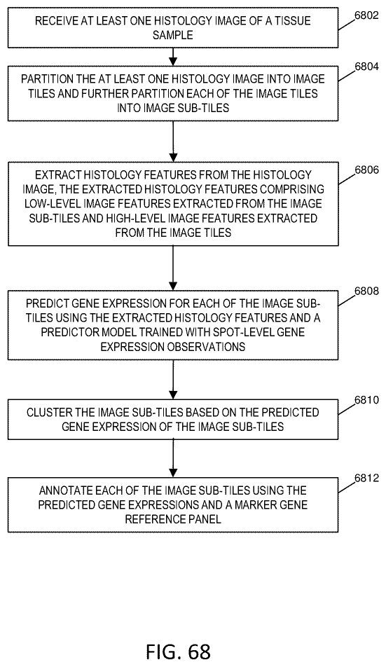

FIG. 68 is a flow diagram illustrating an example method 6800 for inferring super-resolution tissue architecture by integrating spatial transcriptomics with histology. At step 6802, a histology feature extractor receives at least one histology image of a tissue sample. As a preliminary optional step, the histology feature extractor may rescale the histology image such that a defined superpixel of the histology image displays an image of the tissue sample that is approximately a size of a cell, wherein each of the image sub-tiles are sized corresponding to the defined superpixel.

At step 6804, the histology feature extractor partitions the histology image into image tiles and further partitions each of the image tiles into image sub-tiles.

At step 6806, the histology feature extractor extracts histology features from the histology image, the extracted histology features comprising low-level image features extracted from the image sub-tiles and high-level image features extracted from the image tiles. Extracting the histology features from the histology image may include mapping each of the image sub-tiles into a low-level local feature vector, mapping the low-level local features vectors of the image sub-tiles within each of the image tiles into a high-level local feature vector for each of the image tiles, and mapping the high-level local feature vectors into high-level global features. Extracting the histology features from the histology image may include using an extractor model trained by histology datasets. The histology feature extractor can extract the histology features without using any information in spot-level spatial transcriptomic (ST) data, such as spot-level gene expressions. The histology feature extraction procedure can be separate and independent from the gene expression prediction procedure described in following steps. The two-stage approach in extracting high-quality histology features, then integrating the histology features with spot-level ST data to enhance the resolution of the spot-level ST data is significantly more accurate and efficient compared to methods implementing one-stage approaches of simultaneously attempt to improve histology feature extraction and ST resolution enhancement in a single model.

At step 6808, a super-resolution gene expression predictor predicts gene expression for each of the image sub-tiles using the extracted histology features and a predictor model trained with spot-level gene expression observations. The predictor model may include a weakly supervised learning model trained with training data including spot-level gene expression observations. The spot-level gene expression may be modeled as the sum of the gene expressions of the image sub-tiles inside the spot.

At step 6810, a tissue architecture annotator clusters the image sub-tiles based on the predicted gene expression of the image sub-tiles.

At step 6812, the tissue architecture annotator annotates each of the image sub-tiles using the predicted gene expressions and a marker gene reference panel. Annotating each of the image sub-tiles may include determining cell type scores by averaging the super-resolution gene expressions of each cell type's marker genes for each of the image sub-tiles and attributing the cell type with the highest score to the corresponding image sub-tile when the highest score exceeds a threshold. The marker gene reference panel may include user-defined structures and associated marker genes received by the tissue architecture annotator for detecting user-defined structures. The tissue architecture annotator may predict cell type composition for the clusters by determining over-represented cell types within the clusters using the annotated cell types for the image sub-tiles within the clusters.

The at least one histology image of the tissue sample can include a plurality of histology images, wherein each of the histology images is of a distinct tissue slice of the tissue sample. Method 6800 can further include identifying representative histology images of the distinct tissue slices, aligning gene expressions of the representative histology images, and imputing gene expressions between the representative histology images to infer super-resolution tissue architecture (and annotate) a 3D volume of tissue.

The predictor model can be trained with spot-level gene expression observations of at least one training subject, wherein the at least one training subject is distinct from a source of the tissue sample.

The prediction model can be trained with spot-level gene expression observations and transcriptomics data, wherein the super-resolution gene expression predictor predicts gene expression and an omic modality.

The prediction model can be trained with information sourced from a plurality of platforms.

It will be appreciated that method 6800 is for illustrative purposes and that different and/or additional actions may be used. It will also be appreciated that various actions described herein may occur in a different order or sequence. It will be understood that various details of the subject matter described herein may be changed without departing from the scope of the subject matter described herein. Furthermore, the foregoing description is for the purpose of illustration only, and not for the purpose of limitation, as the subject matter described herein is defined by the claims as set forth hereinafter.

### Data Availability

We analyzed the following publicly available ST datasets: (1) 10× Xenium human breast cancer data (https://www.10×genomics.com/products/xenium-in-situ/preview-dataset-human-breast); (2) 10× Xenium mouse brain data (https://www.10×genomics.com/resources/datasets/fresh-frozen-mouse-brain-replicates-1-standard); (3) human HER2-positive breast cancer ST data reported in Anderson et al. (https://github.com/almaan/her2st); (4) 10× Visium human breast cancer data (https://www.10×genomics.com/resources/datasets/human-breast-cancer-visium-fresh-frozen-whole-transcriptome-1-standard); (5) 10× Visium human colorectal cancer data (https://www.10×genomics.com/resources/datasets/human-colorectal-cancer-whole-transcriptome-analysis-1-standard-1-2-0); (6) 10× Visium human prostate cancer data (https://www.10×genomics.com/resources/datasets/human-prostate-cancer-adenocarcinoma-with-invasive-carcinoma-ffpe-1-standard-1-3-0); (7) Human prostate cancer data reported in Erickson et al. (https://doi.org/10.17632/svw96g68dv.1); (8) Human clear cell renal cell carcinoma primary tumors reported in Meylan et al. (GSE175540); (9) 10× Visium mouse kidney data (https://www.10×genomics.com/resources/datasets/adult-mouse-kidney-ffpe-1-standard-1-3-0); (10) 10× Visium mouse brain coronal cut data (https://www.10×genomics.com/resources/datasets/mouse-brain-coronal-section-2-ffpe-2-standard); (11) 10× Visium mouse brain sagittal cut posterior data (https://www.10×genomics.com/resources/datasets/mouse-brain-serial-section-2-sagittal-posterior-1-standard); (12) 10× Visium mouse brain olfactory bulb data (https://www.10×genomics.com/resources/datasets/adult-mouse-olfactory-bulb-1-standard-1). Details of the datasets analyzed in this paper were described in Table 3. Gene expression visualizations for other spatial resolutions in the 10× Xenium breast cancer and mouse brain data are available at https://upenn.box.com/v/istar-results-benchmark.

TABLE 3
Datasets analyzed.

| | | | | | Spot |
|---|---|---|---|---|---|
| | | | | | dia- |
| | | | | | meter |
| | | | Dataset | Pro- | in |
| Species | Tissue | Data source | dimensions | tocol | pixels |
| Human | Breast | 10X Genomics | 167,781 cells | Xenium | N/A |
| | cancer | (https://www.10 | 313 genes | | |
| | | xgenomics.com/ | (Section 1) | | |
| | | products/xenium- | 111,805 cells | | |
| | | in-situ/preview- | 313 genes | | |
| | | dataset-human- | (Section 2) | | |
| | | breast) | | | |
| Mouse | Brain | 10X Genomics | 130,870 Cells | Xenium | N/A |
| | (coronal) | (https://www.10 | 248 genes | | |
| | | xgenomics.com/ | | | |
| | | resources/datas | | | |
| | | ets/fresh-frozen- | | | |
| | | mouse-brain- | | | |
| | | replicates-1- | | | |
| | | standard) | | | |
| Human | HER2- | Andersson et | 441 spots, | Spatial | 400 |
| | positive | al.¹ | 14,992 genes | Trans- | pixels |
| | breast | (https://github.c | (Section G1) | cripto- | |
| | cancer | om/almaan/her2 | 613 spots, | mics | |
| | | st) | 15,029 genes | | |
| | | | (Section H1) | | |
| Human | Breast | 10x Genomics | 4,898 spots, | Visium | 177 |
| | cancer | (https://www.10 | 24,387 genes | | pixels |
| | | xgenomics.com/ | | | |
| | | resources/datas | | | |
| | | ets/human- | | | |
| | | breast-cancer- | | | |
| | | visium-fresh- | | | |
| | | frozen-whole- | | | |
| | | transcriptome-1- | | | |
| | | standard) | | | |
| Human | Colorectal | 10x Genomics | 3,138 spots | Visium | 90 |
| | cancer | (https://www.10 | 23,233 genes | | pixels |
| | | xgenomics.com/ | | | |
| | | resources/datas | | | |
| | | ets/human- | | | |
| | | colorectal- | | | |
| | | cancer-whole- | | | |
| | | transcriptome- | | | |
| | | analysis-1- | | | |
| | | standard-1-2-0) | | | |
| Human | Prostate | 10x Genomics | 4,371 spots | Visium | 188 |
| | cancer | (https://www.10 | 17,943 genes | | pixels |
| | | xgenomics.com/ | | | |
| | | resources/datas | | | |
| | | ets/human- | | | |
| | | prostate-cancer- | | | |
| | | adenocarcinom | | | |
| | | a-with-invasive- | | | |
| | | carcinoma-ffpe- | | | |
| | | 1-standard-1-3- | | | |
| | | 0) | | | |
| Human | Prostate | Erickson et al.² | 4,079 spots | Visium | 113 |
| | Cancer | (https://doi.org/1 | 21,269 genes | | pixels |
| | | 0.17632/svw96g | (Patient 1, | | |
| | | 68dv.1) | Sample H1_4) | | |
| Human | Clear cell | Meylan et al.³ | 1,949 spots | Visium | 142 |
| | renal cell | (GSE175540) | 36,601 genes | | pixels |
| | carcinoma | | | | |
| | primary | | | | |
| | tumors | | | | |
| Mouse | Kidney | 10x Genomics | 3,124 spots | Visium | 188 |
| | | (https://www.10 | 19,465 genes | | pixels |
| | | xgenomics.com/ | | | |
| | | resources/datas | | | |
| | | ets/adult- | | | |
| | | mouse-kidney- | | | |
| | | ffpe-1-standard- | | | |
| | | 1-3-0) | | | |
| Mouse | Brain | 10x Genomics | 2,235 spots | Visium | 256 |
| | (coronal) | (https://www.10 | 19,464 genes | | pixels |
| | | xgenomics.com/ | | | |
| | | resources/datas | | | |
| | | ets/mouse- | | | |
| | | brain-coronal- | | | |
| | | section-2-ffpe-2- | | | |
| | | standard) | | | |
| Mouse | Posterior | 10x Genomics | 3,289 spots | Visium | 90 |
| | brain | (https://www.10 | 32,285 genes | | pixels |
| | (sagittal) | xgenomics.com/ | | | |
| | | resources/datas | | | |
| | | ets/mouse- | | | |
| | | brain-serial- | | | |
| | | section-2- | | | |
| | | sagittal- | | | |
| | | posterior-1- | | | |
| | | standard) | | | |
| Mouse | Brain | 10x Genomics | 1,185 spots | Visium | 74 |
| | (olfactory | (https://www.10 | 32,285 genes | | pixels |
| | bulb) | xgenomics.com/ | | | |
| | | resources/datas | | | |
| | | ets/adult- | | | |
| | | mouse- | | | |
| | | olfactory-bulb-1- | | | |
| | | standard-1) | | | |

The disclosure of each of the following references is incorporated by reference herein in its entirety:

### REFERENCES

1. Burgess, D. J. Spatial transcriptomics coming of age. Nat Rev Genet 20, 317 (2019).
2. Asp, M., Bergenstrahle, J. & Lundeberg, J. Spatially Resolved Transcriptomes-Next Generation Tools for Tissue Exploration. Bioessays 42, e1900221 (2020).
3. Crosetto, N., Bienko, M. & van Oudenaarden, A. Spatially resolved transcriptomics and beyond. Nat Rev Genet 16, 57-66 (2015).
4. Moor, A. E. & Itzkovitz, S. Spatial transcriptomics: paving the way for tissue-level systems biology. Curr Opin Biotechnol 46, 126-133 (2017).
5. Hu, J. et al. SpaGCN: Integrating gene expression, spatial location and histology to identify spatial domains and spatially variable genes by graph convolutional network. Nat Methods 18, 1342-1351 (2021).
6. Sun, S., Zhu, J. & Zhou, X. Statistical analysis of spatial expression patterns for spatially resolved transcriptomic studies. Nat Methods 17, 193-200 (2020).
7. Svensson, V., Teichmann, S. A. & Stegle, O. SpatialDE: identification of spatially variable genes. Nat Methods 15, 343-346 (2018).
8. Dries, R. et al. Giotto: a toolbox for integrative analysis and visualization of spatial expression data. Genome Biol 22, 78 (2021).
9. Pham, D. et al. stLearn: integrating spatial location, tissue morphology and gene expression to find cell types, cell-cell interactions and spatial trajectories within undissociated tissues. bioRxiv (2020).
10. Asp, M. et al. A Spatiotemporal Organ-Wide Gene Expression and Cell Atlas of the Developing Human Heart. Cell 179, 1647-1660 e1619 (2019).
11. Wang, X. et al. Three-dimensional intact-tissue sequencing of single-cell transcriptional states. Science 361, eaat5691 (2018).
12. Lubeck, E., Coskun, A. F., Zhiyentayev, T., Ahmad, M. & Cai, L. Single-cell in situ RNA profiling by sequential hybridization. Nat Methods 11, 360-361 (2014).
13. Shah, S., Lubeck, E., Zhou, W. & Cai, L. In Situ Transcription Profiling of Single Cells Reveals Spatial Organization of Cells in the Mouse Hippocampus. Neuron 92, 342-357 (2016).
14. Eng, C. L. et al. Transcriptome-scale super-resolved imaging in tissues by RNA seqFISH. Nature 568, 235-239 (2019).
15. Moffitt, J. R. et al. Molecular, spatial, and functional single-cell profiling of the hypothalamic preoptic region. Science 362 (2018).
16. Chen, K. H., Boettiger, A. N., Moffitt, J. R., Wang, S. & Zhuang, X. RNA imaging. Spatially resolved, highly multiplexed RNA profiling in single cells. Science 348, aaa6090 (2015).
17. Stickels, R. R. et al. Highly sensitive spatial transcriptomics at near-cellular resolution with Slide-seqV2. Nat Biotechnol (2020).
18. Vickovic, S. et al. High-definition spatial transcriptomics for in situ tissue profiling. Nat Methods 16, 987-990 (2019).
19. Liu, Y. et al. High-Spatial-Resolution Multi-Omics Sequencing via Deterministic Barcoding in Tissue. Cell 183, 1665-1681 e1618 (2020).
20. Chen, A. et al. Spatiotemporal transcriptomic atlas of mouse organogenesis using DNA nanoball-patterned arrays. Cell 185, 1777-1792 e1721 (2022).
21. Badea, L. & Stanescu, E. Identifying transcriptomic correlates of histology using deep learning. PLoS One 15, e0242858 (2020).
22. Ash, J. T., Darnell, G., Munro, D. & Engelhardt, B. E. Joint analysis of expression levels and histological images identifies genes associated with tissue morphology. Nat Commun 12, 1609 (2021).
23. Schmauch, B. et al. A deep learning model to predict RNA-Seq expression of tumours from whole slide images. Nat Commun 11, 3877 (2020).
24. Chen, R. J. et al. in Proceedings of the IEEE/CVF Conference on Computer Vision and Pattern Recognition 16144-16155 (2022).
25. Liu, Z. et al. in Proceedings of the IEEE/CVF international conference on computer vision 10012-10022 (2021).
26. Han, K. et al. Transformer in transformer. Advances in Neural Information Processing Systems 34, 15908-15919 (2021).
27. Caron, M. et al. in Proceedings of the IEEE/CVF international conference on computer vision 9650-9660 (2021).
28. Bao, H., Dong, L., Piao, S. & Wei, F. Beit: Bert pre-training of image transformers. arXiv preprint arXiv: 2106.08254 (2021).
29. Janesick, A. et al. High resolution mapping of the breast cancer tumor microenvironment using integrated single cell, spatial and in situ analysis of FFPE tissue. bioRxiv, 2022.2010. 2006.510405 (2022).
30. Bergenstråhle, L. et al. Super-resolved spatial transcriptomics by deep data fusion. Nature biotechnology 40, 476-479 (2022).
31. Wang, Z., Bovik, A. C., Sheikh, H. R. & Simoncelli, E. P. Image quality assessment: from error visibility to structural similarity. IEEE transactions on image processing 13, 600-612 (2004).
32. Hamerly, G. & Elkan, C. Learning the k in k-means. Advances in neural information processing systems 16 (2003).
33. Andersson, A. et al. Spatial deconvolution of HER2-positive breast cancer delineates tumor-associated cell type interactions. Nature communications 12, 6012 (2021).
34. Stahl, P. L. et al. Visualization and analysis of gene expression in tissue sections by spatial transcriptomics. Science 353, 78-82 (2016).
35. Wu, S. Z. et al. A single-cell and spatially resolved atlas of human breast cancers. Nature genetics 53, 1334-1347 (2021).
36. Sautes-Fridman, C., Petitprez, F., Calderaro, J. & Fridman, W. H. Tertiary lymphoid structures in the era of cancer immunotherapy. Nat Rev Cancer 19, 307-325 (2019).
37. Fridman, W. H. et al. B cells and tertiary lymphoid structures as determinants of tumour immune contexture and clinical outcome. Nat Rev Clin Oncol 19, 441-457 (2022).
38. Helmink, B. A. et al. B cells and tertiary lymphoid structures promote immunotherapy response. Nature 577, 549-555 (2020).
39. Petitprez, F. et al. B cells are associated with survival and immunotherapy response in sarcoma. Nature 577, 556-560 (2020).
40. Cabrita, R. et al. Tertiary lymphoid structures improve immunotherapy and survival in melanoma. Nature 577, 561-565 (2020).
41. Lin, J. R. et al. Multiplexed 3D atlas of state transitions and 584 immune interaction in colorectal cancer. Cell 186, 363-381 e319 (2023).
42. Steiner, A. et al. How to train your vit? data, augmentation, and regularization in vision transformers. arXiv preprint arXiv: 2106.10270 (2021).
43. Hölscher, D. L. et al. Next-Generation Morphometry for pathomics-data mining in histopathology. Nature Communications 14, 470 (2023).
44. Xu, B., Wang, N., Chen, T. & Li, M. Empirical evaluation of rectified activations in convolutional network. arXiv preprint arXiv: 1505.00853 (2015).
45. Clevert, D. A., Unterthiner, T. & Hochreiter, S. Fast and accurate deep network learning by exponential linear units (elus). arXiv preprint arXiv: 1511.07289 (2015).
46. Schurch, C. M. et al. Coordinated Cellular Neighborhoods Orchestrate Antitumoral Immunity at the Colorectal Cancer Invasive Front. Cell 182, 1341-1359 e1319 (2020).
47. Aran, D. et al. Reference-based analysis of lung single-cell sequencing reveals a transitional profibrotic macrophage. Nature immunology 20, 163-172 (2019).
48. Hu, J. et al. Iterative transfer learning with neural network for clustering and cell type classification in single-cell RNA-seq analysis. Nature machine intelligence 2, 607-618 (2020).
49. Lu, Y. in Proceedings of the AAAI Conference on Artificial Intelligence, Vol. 33 9989-9990 (2019).
50. Lai, W. S., Huang, J. B., Ahuja, N. & Yang, M. H. in Proceedings of the IEEE conference on computer vision and pattern recognition 624-632 (2017).
51. Dahl, R., Norouzi, M. & Shlens, J. in Proceedings of the IEEE international conference on computer vision 5439-5448 (2017).
52. Masutani, E. M., Bahrami, N. & Hsiao, A. Deep learning single-frame and multiframe super-resolution for cardiac MRI. Radiology 295, 552-561 (2020).
53. Anwar, S., Khan, S. & Barnes, N. A deep journey into super-resolution: A survey. ACM Computing Surveys (CSUR) 53, 1-34 (2020).
54. Wang, Z., Chen, J. & Hoi, S. C. Deep learning for image super-resolution: A survey. IEEE transactions on pattern analysis and machine intelligence 43, 3365-3387 (2020).
55. Ma, Y. & Zhou, X. Spatially informed cell-type deconvolution for spatial transcriptomics. Nature biotechnology 40, 1349-1359 (2022).
56. Andersson, A. et al. Spatial deconvolution of HER2-positive breast cancer delineates tumor-associated cell type interactions. Nat Commun 12, 6012 (2021).
57. Erickson, A. et al. Spatially resolved clonal copy number alterations in benign and malignant tissue. Nature 608, 360-367 (2022).
58. Meylan, M. et al. Tertiary lymphoid structures generate and propagate anti-tumor antibody-producing plasma cells in renal cell cancer. Immunity 55, 527-541 e525 (2022).
59. Wu, S. Z. et al. A single-cell and spatially resolved atlas of human breast cancers. Nat Genet 53, 1334-1347 (2021).
60. Qi, J. et al. Single-cell and spatial analysis reveal interaction of FAP (+) fibroblasts and SPP1 (+) macrophages in colorectal cancer. Nat Commun 13, 1742 (2022).

What is claimed is:

1. A method for inferring super-resolution tissue architecture by integrating spatial transcriptomics with histology, the method comprising:
receiving, at a histology feature extractor, at least one histology image of a tissue sample;
partitioning, by the histology feature extractor, each of the at least one histology image into image tiles and further partitioning each of the image tiles into image sub-tiles;
extracting, by the histology feature extractor, histology features from the histology image, the extracted histology features comprising low-level image features extracted from the image sub-tiles and high-level image features extracted from the image tiles;
predicting, by a super-resolution gene expression predictor, gene expression for each of the image sub-tiles using the extracted histology features and a predictor model trained with spot-level gene expression observations;
clustering, by a tissue architecture annotator, the image sub-tiles based on the predicted gene expression of the image sub-tiles; and
annotating, by the tissue architecture annotator, each of the image sub-tiles using the predicted gene expressions and a marker gene reference panel.

2. The method of claim 1 comprising predicting, by the super-resolution gene expression predictor, single cell-level gene expressions using cell segmentation masks and the predicted sub-tile level gene expressions.

3. The method of claim 1 wherein extracting the histology features from the histology image comprises mapping each of the image sub-tiles into a low-level local feature vector, mapping the low-level local features vectors of the image sub-tiles within each of the image tiles into a high-level local feature vector for each of the image tiles, and mapping the high-level local feature vectors into high-level global features.

4. The method of claim 1 wherein extracting the histology features from the histology image comprises using an extractor model trained by histology datasets.

5. The method of claim 1 wherein the predictor model comprises a weakly supervised learning model trained with training data comprising spot-level gene expression observations.

6. The method of claim 5 wherein the spot-level gene expression is modeled as the sum of the gene expressions of the image sub-tiles inside the spot.

7. The method of claim 1 wherein annotating each of the image sub-tiles comprises determining cell type scores by averaging the super-resolution gene expressions of each cell type's marker genes for each of the image sub-tiles and attributing the cell type with the highest score to the corresponding image sub-tile when the highest score exceeds a threshold.

8. The method of claim 7 wherein the marker gene reference panel includes user-defined structures and associated marker genes received by the tissue architecture annotator for detecting user-defined structures.

9. The method of claim 1 comprising predicting cell type composition for the clusters by determining over-represented cell types within the clusters using the annotated cell types for the image sub-tiles within the clusters.

10. The method of claim 1 wherein the at least one histology image of the tissue sample includes a plurality of histology images, wherein each of the histology images is of a distinct tissue slice of the tissue sample, wherein the method comprises identifying representative histology images of the distinct tissue slices, aligning gene expressions of the representative histology images, and imputing gene expressions between the representative histology images.

11. The method of claim 1 wherein the predictor model is trained with spot-level gene expression observations of at least one training subject, wherein the at least one training subject is distinct from a source of the tissue sample.

12. The method of claim 1 wherein the prediction model is trained with spot-level gene expression observations and transcriptomics data, wherein the super-resolution gene expression predictor predicts gene expression and an omic modality.

13. The method of claim 1 wherein the prediction model is trained with information sourced from a plurality of platforms.

14. A system for inferring super-resolution tissue architecture by integrating spatial transcriptomics with histology, the system comprising:
at least one processor and a memory; and
a histology feature extractor implemented by the at least one processor and configured for:
receiving a histology image of a tissue sample;
partitioning the histology image into image tiles and further partitioning each of the image tiles into image sub-tiles; and
extracting histology features from the histology image, the extracted histology features comprising low-level image features extracted from the image sub-tiles and high-level image features extracted from the image tiles;
a super-resolution gene expression predictor configured for:
predicting gene expression for each of the image sub-tiles using the extracted histology features and a predictor model trained with spot-level gene expression observations; and
a tissue architecture annotator configured for:
clustering the image sub-tiles based on the predicted gene expression of the image sub-tiles; and
annotating each of the image sub-tiles using the predicted gene expressions and a marker gene reference panel.

15. The system of claim 14 wherein the super-resolution gene expression predictor is configured for predicting single cell-level gene expressions using cell segmentation masks and the predicted sub-tile level gene expressions.

16. The system of claim 14 wherein extracting the histology features from the histology image comprises mapping each of the image sub-tiles into a low-level local feature vector, mapping the low-level local features vectors of the image sub-tiles within each of the image tiles into a high-level local feature vector for each of the image tiles, and mapping the high-level local feature vectors into high-level global features.

17. The system of claim 14 wherein extracting the histology features from the histology image comprises using an extractor model trained by histology datasets.

18. The system of claim 14 wherein the predictor model comprises a weakly supervised learning model trained with training data comprising spot-level gene expression observations.

19. The system of claim 18 wherein the spot-level gene expression is modeled as the sum of the gene expressions of the image sub-tiles inside the spot.

20. The system of claim 14 wherein annotating each of the image sub-tiles comprises determining cell type by averaging the super-resolution gene expressions of each cell type's marker genes for each of the image sub-tiles and attributing the cell type with the highest score to the corresponding image sub-tile when the highest score exceeds a threshold.

21. The system of claim 20 wherein the marker gene reference panel includes user-defined structures and associated marker genes received by the tissue architecture annotator for annotating user-defined structures.

22. The system of claim 14 wherein the tissue architecture annotator is configured for predicting cell type composition for the clusters by determining over-represented cell types within the clusters using the annotated cell types for the image sub-tiles within the clusters.

23. The system of claim 14 wherein the at least one histology image of the tissue sample includes a plurality of histology images, wherein each of the histology images is of a distinct tissue slice of the tissue sample, wherein the system is further configured for identifying representative histology images of the distinct tissue slices, aligning gene expressions of the representative histology images, and imputing gene expressions between the representative histology images.

24. The system of claim 14 wherein the predictor model is trained with spot-level gene expression observations of at least one training subject, wherein the at least one training subject is distinct from a source of the tissue sample.

25. The system of claim 14 wherein the prediction model is trained with spot-level gene expression observations and transcriptomics data, wherein the super-resolution gene expression predictor predicts gene expression and an omic modality.

26. The system of claim 14 wherein the prediction model is trained with information sourced from a plurality of platforms.

27. A non-transitory computer readable medium having stored thereon executable instructions that when executed by at least one processor of at least one computer cause the at least one computer to perform steps comprising:
receiving a histology image of a tissue sample;
partitioning the histology image into image tiles and further partitioning each of the image tiles into image sub-tiles;
extracting histology features from the histology image, the extracted histology features comprising low-level image features extracted from the image sub-tiles and high-level image features extracted from the image tiles;
predicting gene expression for each of the image sub-tiles using the extracted histology features and a predictor model trained with spot-level gene expression observations;
clustering the image sub-tiles based on the predicted gene expression of the image sub-tiles; and
annotating each of the image sub-tiles using the predicted gene expressions and a marker gene reference panel.

28. The non-transitory computer readable medium of claim 27 wherein the non-transitory computer readable medium is configured for predicting single cell-level gene expressions using cell segmentation masks and the predicted sub-tile level gene expressions.
